(19) United States
(12) Patent Application Publication
(10) Pub. No.: US 2023/0295606 A1
(43) Pub. Date: Sep. 21, 2023

(54) LIGATION FREE METHODS OF NUCLEIC ACID LIBRARY PREPARATION

(71) Applicant: Arc Bio, LLC, Cambridge, MA (US)

(72) Inventors: Jannine FORST, San Jose, CA (US); Morten RASMUSSEN, Mountain View, CA (US); Stephane B. GOURGUECHON, San Mateo, CA (US)

(21) Appl. No.: 17/782,841

(22) PCT Filed: Nov. 30, 2020
(86) PCT No.: PCT/US2020/062592

§ 371 Date: Jun. 6, 2022

Related U.S. Application Data
(60) Provisional application No. 62/945,051, filed on Dec. 6, 2019.

(51) Int. Cl. C12N 15/10

(52) U.S. Cl. CPC C12N 15/1065; C12N 15/1068

(57) ABSTRACT

Provided herein are compositions and methods for generating libraries for high throughput sequencing without using ligation, and methods of using same.

### STATEMENT OF GOVERNMENT INTEREST

This invention was made with government support under 2017_DN_BX_0140 awarded by the National Institute of Justice. The government has certain rights in the invention.

### BACKGROUND

Conventional techniques of preparing libraries of nucleic acids for high throughput sequencing use ligation to introduce adapters onto the 5′ and 3′ ends of the nucleic acids. However, these techniques may not be suitable for small and/or highly degraded samples. There thus exists a need in the art for additional, ligation-free methods of library preparation. The disclosure provides ligation-free methods of library preparation suitable for small and/or highly degraded samples.

### SUMMARY

The disclosure provides methods of preparing a library of nucleic acids, comprising: (a) providing a sample of nucleic acids comprising at least one sequence of interest; (b) contacting the sample of nucleic acids with a plurality of first polymerase chain reaction (PCR) primers, and a first polymerase under conditions that allow PCR to occur, thereby generating a plurality of first single-sided PCR products; (c) contacting the plurality of first single-sided PCR products with a terminal transferase and dNTPs under conditions sufficient to transfer dNTPs to the 3′ ends of the plurality of first single-sided PCR products, thereby generating a plurality of PCR products comprising 3′ tails; and (d) contacting the plurality of PCR products comprising 3′ tails with a plurality of second PCR primers, and the first polymerase under conditions that allow PCR to occur; thereby generating a library of nucleic acids with adapters at the 5′ and 3′ ends.

The disclosure provides methods of preparing a library of nucleic acids, comprising: (a) providing a sample of nucleic acids comprising at least one sequence of interest; (b) contacting the sample of nucleic acids with a terminal transferase and dNTPs under conditions sufficient to transfer dNTPs to the 3′ ends of the nucleic acids, thereby generating a sample of nucleic acids comprising 3′ tails; (c) contacting the sample of nucleic acids comprising 3′ tails with a plurality of first polymerase chain reaction (PCR) primers, and a first polymerase under conditions that allow PCR to occur, thereby generating a plurality of first single-sided PCR products; and (d) contacting the plurality of first single-sided PCR products with a plurality of second PCR primers, and the first polymerase under conditions that allow PCR to occur; thereby generating a library of nucleic acids with adapters at the 5′ and 3′ ends.

In some embodiments of the methods of the disclosure, the methods comprise contacting the library of nucleic acids from (d) with a plurality of first indexing primers, a plurality of second indexing primers and a second polymerase under conditions that allow PCR to occur.

In some embodiments of the methods of the disclosure, the sample comprises nucleic acids of interest and nucleic acids targeted for depletion, and at least a subset of the nucleic acids targeted for depletion comprise a plurality of recognition sites for a modification-sensitive restriction enzyme. In some embodiments, the methods comprise terminally dephosphorylating a plurality of the nucleic acids in the sample. In some embodiments, the methods comprise contacting the sample with the modification-sensitive restriction enzyme under conditions that allow for the cleavage of the modification-sensitive restriction sites in the nucleic acids in the sample, thereby generating nucleic acids with exposed terminal phosphates; and contacting the sample with an exonuclease under conditions that allow for the successive removal of nucleotides from a phosphorylated end of a nucleic acid; thereby generating a sample enriched for nucleic acids of interest. In some embodiments, the methods comprise comprising contacting the sample of nucleic acids with a modification-sensitive restriction enzyme under conditions that allow for the cleavage of the modification-sensitive restriction sites in the nucleic acids in the sample; thereby generating a sample enriched for nucleic acids of interest that have adapters on their 5′ and 3′ ends.

The disclosure provides methods of preparing a library of nucleic acids, comprising (a) providing a sample of nucleic acids comprising at least one sequence of interest; (b) contacting the sample of nucleic acids with a terminal transferase and NTPs under conditions sufficient to transfer NTPs to the 3′ end of the nucleic acids thereby generating a plurality of nucleic acids comprising 3′ tails; (c) contacting the plurality of nucleic acids comprising 3′ tails with a plurality of first adapters and a reverse transcriptase under conditions sufficient for first strand complementary DNA (cDNA) synthesis to occur, thereby generating a plurality of cDNAs, wherein the plurality of cDNAs comprise 3′ polyC sequences; and (d) contacting the plurality of cDNAs with a second adapter under conditions sufficient to allow generation of double stranded DNA from the plurality of cDNAs to generate a plurality of double stranded DNAs, thereby preparing a library of nucleic acids with adapters at the 5′ and 3′ ends.

In some embodiments of the methods of the disclosure, the sequence of interest comprises a single nucleotide polymorphism (SNP), a miniSTR (mini short tandem repeat), a mitochondrial marker, a Y chromosome marker, or a disease trait marker. In some embodiments, the disease trait marker comprises a marker for pathogenicity, virulence, resistance or strain identification.

In some embodiments of the methods of the disclosure, the sample is degraded. In some embodiments, the sample is a forensics sample. In some embodiments,

The disclosure provides methods of preparing a library of nucleic acids comprising: (a) providing a plurality of guide nucleic acid (gNA)-CRISPR/Cas system protein complexes, wherein the gNAs are configured to hybridize to at least one sequence targeted for depletion; (b) mixing the library of nucleic acids with the plurality of gNA-CRISPR/Cas system protein complexes, wherein at least a portion of the gNA-CRISPR/Cas system protein complexes hybridize to the at least one sequence targeted for depletion; and (c) incubating the mixture to cleave the at least one sequence targeted for depletion. In some embodiments, the methods comprise PCR amplifying the library of nucleic acids following step (c).

### BRIEF DESCRIPTION OF THE DRAWINGS

FIG. 1 illustrates an exemplary scheme for the library generation and enrichment in a single workflow.

FIG. 2 illustrates an exemplary schematic of a strand-switching method.

FIG. 3 is an Agilent High Sensitivity D1000 gel illustrating the DNA fragment distribution of ligation free sequencing libraries following indexing and purification, and an A-tailing negative control sample. At top, the wells from left to right are: EL1 (ladder), A1 (iPCR1-Pur-Neg, “Negative” sample), B1 (iPCR1-Pur-Test, “Test” Sample), C1 (iPCR1-Pur-Pos, “Positive” Sample) and D1 (PCR10-Atail-Neg, the A-tailing Negative Control).

FIG. 4 is a plot illustrating the size (x-axis, in base pairs [bp]) and intensity (y-axis, normalized fluorescence units, abbreviated FU) of the ladder (EL1). Lines and brackets indicate regions used to calculate the parameters disclosed in Table 14.

FIG. 5A is a plot illustrating the size (x-axis, in bp) and intensity (y-axis, FU) of the Negative sample (iPCR1-Pur-Neg) following indexing and purification. Lines and brackets indicate regions used to calculate the parameters disclosed in Table 15.

FIG. 5B is a plot illustrating the size (x-axis, in bp) and intensity (y-axis, FU) of the Negative sample (iPCR1-Pur-Neg) following indexing and purification. Lines and brackets indicate regions used to calculate the parameters disclosed in Table 16.

FIG. 6A is a plot illustrating the size (x-axis, in bp) and intensity (y-axis, FU) of the Test sample (iPCR1-Pur-Test) following indexing and purification. Lines and brackets indicate regions used to calculate the parameters disclosed in Table 17.

FIG. 6B is a plot illustrating the size (x-axis, in bp) and intensity (y-axis, FU) of the Test sample (iPCR1-Pur-Test) following indexing and purification. Lines and brackets indicate regions used to calculate the parameters disclosed in Table 18. Dark lines indicate from 100-1000 bp, light lines indicate from 265-1000 bp.

FIG. 7A is a plot illustrating the size (x-axis, in bp) and intensity (y-axis, FU) of the Positive sample (iPCR1-Pur-Pos) following indexing and purification. Lines and brackets indicate regions used to calculate the parameters disclosed in Table 19.

FIG. 7B is a plot illustrating the size (x-axis, in bp) and intensity (y-axis, FU) of the Positive sample (iPCR1-Pur-Pos) following indexing and purification. Lines and brackets indicate regions used to calculate the parameters disclosed in Table 20. Dark lines indicate from 100-1000 bp, light lines indicate from 265-1000 bp.

FIG. 8A is a plot illustrating the size (x-axis, in bp) and intensity (y-axis, FU) of the A-tailing negative sample (PCR10-Atail-Neg). Lines and brackets indicate regions used to calculate the parameters disclosed in Table 21.

FIG. 8B is a plot illustrating the size (x-axis, in bp) and intensity (y-axis, FU) of the A-tailing negative sample (PCR10-Atail-Neg). Lines and brackets indicate regions used to calculate the parameters disclosed in Table 22. Dark lines indicate from 100-1000 bp, light lines indicate from 265-1000 bp.

FIG. 9A is an Agilent High Sensitivity D1000 gel illustrating a profile comparison of A1 (iPCR1-Pur-Neg, “Negative” sample), B1 (iPCR1-Pur-Test, “Test” Sample), C1 (iPCR1-Pur-Pos, “Positive” Sample).

FIG. 9B is a plot illustrating a profile comparison of A1 (iPCR1-Pur-Neg, “Negative” sample, green), B1 (iPCR1-Pur-Test, “Test” Sample, orange), C1 (iPCR1-Pur-Pos, “Positive” Sample, blue). Size in bp is plotted on the x-axis, sample intensity (Normalized FU) is plotted on the y-axis.

FIG. 10 is a plot illustrating the distribution of fragment sizes (read lengths) from high throughput sequencing of the Test and Positive samples.

FIG. 11A is a plot illustrating the sequence counts for the Positive and Test samples. Duplicate read counts are an estimate only.

FIG. 11B is a plot illustrating the percentage of Unique and Duplicate Reads for the Positive and Test samples. Duplicate read counts are an estimate only.

FIG. 12 is a plot illustrating the mean sequence quality value across each base position in the read. The Test sample is shown in dark gray; the Positive sample is shown in light gray.

FIG. 13 is a plot illustrating the number of reads with average quality scores. This shows if a subset of reads have poor quality. The Positive sample is the top line, the Test sample is the lower line.

FIG. 14 is a plot illustrating the proportion of each base position for which each of the four normal DNA bases has been called during sequence analysis. Medium gray: % T; dark gray: % C; light gray: % A and Black: % G.

FIG. 15 is a plot illustrating the per sequence GC content, i.e. the average GC content of reads. Normal random libraries typically have a roughly normal distribution of GC content. The Positive sample is shown in light gray (top peak), the Test sample is shown in dark gray (bottom peak).

FIG. 16 is a plot showing the percentage of base calls at each position for which “N” was called.

FIG. 17 is a plot illustrating the sequence duplication levels. The plot shows the relative level of duplication found for every sequence.

FIG. 18 is a plot illustrating the total amount of over-represented sequences found in each library.

FIG. 19 is a diagram illustrating an exemplary method of the invention. Nucleic acids in the sample are dephosphorylated, and then digested with a restriction enzyme that is blocked by the presence of modifications at the restriction enzyme recognition site. The exposed phosphates from the resulting digestion are then used to ligate adapters to the nucleic acids of interest.

FIG. 20 is a diagram illustrating an exemplary method of the invention. Nucleic acids in the sample are dephosphorylated, and then digested with a restriction enzyme that recognizes a restriction enzyme site comprising one or more modified nucleotides. Cut nucleic acids are then digested with an exonuclease that uses the exposed terminal phosphates, and adapters are ligated to the remaining nucleic acids of interest.

FIG. 21 is a diagram illustrating an exemplary method of the invention. Nucleic acids in the sample have adapters attached, and are then digested with a restriction enzyme that recognizes a restriction enzyme site comprising one or more modified nucleotides, resulting in nucleic acids of interest that have adapters on both ends.

FIG. 22 is a diagram illustrating an exemplary method of the disclosure. Nucleic acids in the sample have adapters on both ends, and are then cleaved with a nucleic acid-guided nuclease that cleaves the nucleic acids targeted for depletion, resulting in nucleic acids of interest that have adapters on both ends. This method can be used in conjunction with the nucleotide modification-based methods of the disclosure.

FIG. 23 is a plot showing the number of reads passing filters for quality and length from libraries generated using ligation-free preparation methods.

FIG. 24 is a plot showing the number of reads mapped to a reference genome out of the number of useable reads from libraries generated using ligation-free preparation methods.

FIG. 25 is a plot showing the number of unique molecules (i.e., the rate of duplication) for reads rom libraries generated using ligation-free preparation methods.

FIG. 26 is a plot showing a comparison of performance with different G-tail lengths.

FIG. 27 is a plot showing the ability of ligation free methods of library preparation to capture rare molecules.

### DETAILED DESCRIPTION OF THE INVENTION

Capturing information from trace nucleic acid samples, or degraded samples comprising small nucleic acid fragments, remains a significant challenge, particularly for the field of DNA forensics. These samples generally contain nucleic acid fragments that are too small for traditional PCR. Further, the amount of nucleic acids in the sample may be too small for traditional ligation-based based methods library preparation, which are inefficient. However, high-throughput sequencing (HTS) has the potential to recover information from these samples, as even small fragments can contain single nucleotide polymorphisms (SNPs) or other markers useful for identification, predicting visible characteristics such as ancestry and hair/eye color, and generating investigative leads. Disclosed herein are methods of ligation-free library preparation that can be optionally combined with targeted enrichment and/or depletion strategies that, coupled with custom informatics methods, can generate investigative leads from highly-degraded forensic samples.

The disclosure provides methods of preparing libraries of nucleic acids, sometimes referred to herein as collections, without ligating adapters to the nucleic acids. The ligation-free methods of the instant disclosure allow for the capture of small fragments (e.g., less than 50 bp) in libraries, e.g. sequencing libraries. Thus, the methods of the instant disclosure are superior in their ability to capture small, trace and/or highly degraded nucleic acid samples in sequencing libraries for analysis when compared to convention methods of library preparation, which rely on adapter ligation. The libraries described herein can be used for sequencing, including high-throughput sequencing.

The term “next-generation sequencing” refers to the so-called parallelized sequencing-by-synthesis or sequencing-by-ligation platforms, for example, those currently employed by Illumina, Life Technologies, and Roche, etc. Next-generation sequencing methods may also include nanopore sequencing methods or electronic-detection based methods such as Ion Torrent technology commercialized by Life Technologies.

Capturing information from trace and degraded nucleic acid samples remains a significant challenge, particularly for the field of DNA forensics, but also for other fields such as archaeology and ancient DNA, and the analysis of cell-free nucleic acids. These samples generally contain nucleic acids in fragments that are too small for traditional PCR and are thus not amenable to Combined DNA Index System (CODIS) profiling. Furthermore, the samples may not even contain complete copies of the donor's genome. High-throughput sequencing has the potential to recover information from these samples, as even small fragments can contain single nucleotide polymorphisms (SNPs) or other markers useful for identification, predicting visible characteristics such as ancestry and hair/eye color, and generating investigative leads.

Disclosed herein are methods of ligation-free library preparation that can be optionally combined with a targeted enrichment strategy or depletion strategy that, coupled with custom informatics methods, can generate investigative leads from highly-degraded forensic samples.

In some embodiments, the methods of the disclosure comprise (a) extracting nucleic acids using a protocol optimized to retain small fragments; (b) applying one of the ligation-free library preparation methods disclosed herein, wherein the method is targeted to a pre-selected panel of forensically relevant SNPs; (c) sequencing the library with high-throughput sequence methods; and (d) using custom informatics methods to generate a report that includes sex, autosomal ancestry, maternal and paternal lineage, select phenotypic markers, and match probabilities with confidence levels. In some embodiments, the library prepared using the ligation-free methods described herein is subject to depletion of sequences targeted for depletion prior to sequencing, thereby enriching for sequences of interest. For example, a sequencing library from a human forensics sample can be contacted with a plurality of gNAs and CRISPR/Cas system proteins prior to sequencing, wherein the plurality of gNAs target sequences for depletion, for example, human sequences excluding sequences comprising forensically relevant SNPs or other markers.

### Ligation-Free Preparation of Nucleic Acids by Single-Sided PCR

Libraries of nucleic acids of the disclosure can be prepared through the use of single-sided PCR, sometimes referred to as one-sided PCR or asymmetric PCR. Single-sided PCR uses a single primer that hybridizes to a target sequence in a single stranded nucleic acid molecule and extends along the template to produce a double-stranded product. Single-sided PCR can be used to add additional sequences, such as adapters, to the double-stranded PCR product, for example by including these additional sequences on the 5′ end of the primer used to prime the single-sided PCR reaction. In some embodiments, the methods comprise providing a sample of nucleic acids, introducing a first adapter to the nucleic acids in the sample using a first single-sided PCR reaction, and introducing a second adapter to the nucleic acids in the sample using a second single-sided PCR reaction.

In some embodiments, the methods described herein comprise targeted primer extension-based sequencing methods using single-sided PCR. The targeted primer extension-based sequencing methods of the disclosure involve the use of a single primer binding near a sequence of interest (for example, a SNP or miniSTR). This approach bypasses the need for two primer binding sites in a fragment (e.g., in PCR), enabling the inclusion of very small (<50 base pair) fragments. Furthermore, sequencing adapters are added without the need for ligation, which is known to be highly inefficient and results in sample loss.

Sequencing using the methods described herein can be conducted without ligation of adapters. This can enable sequencing of otherwise difficult to sequence samples, such as highly degraded samples. Highly degraded DNA, in addition to containing primarily short fragments, often has cross-links to other molecules, making the end-to-end amplification required for sequencing libraries inefficient or impossible. Additionally, existing protocols can require conversion of the entire sample to DNA libraries by ligating adapters, followed by a time-consuming enrichment and multiple PCR amplifications.

Such ligation-free library preparation protocols can be used for forensics or other identification of individuals. For example, sequences of interest can include SNPs and other markers in mitochondrial DNA (mtDNA) and Y chromosome sites for assignment of maternal and paternal haplogroups. MiniSTRs or other identifying regions can be employed. For degraded samples, it is often favorable to look at the mitochondrial DNA due to its high copy number and well-characterized haplogroup tree.

Such ligation-free library preparation protocols can be used for disease diagnostics. For example, sequences of interest can include taxonomic markers including clade markers. Sequences of interest can include disease trait markers such as pathogenicity, virulence, resistance, strain identification, and other markers.

The pipeline described herein can be applied to extract information from samples for which the Combined DNA Index System (CODIS) genotyping failed, and can also provide investigative leads for cases in which no match is found in the CODIS database.

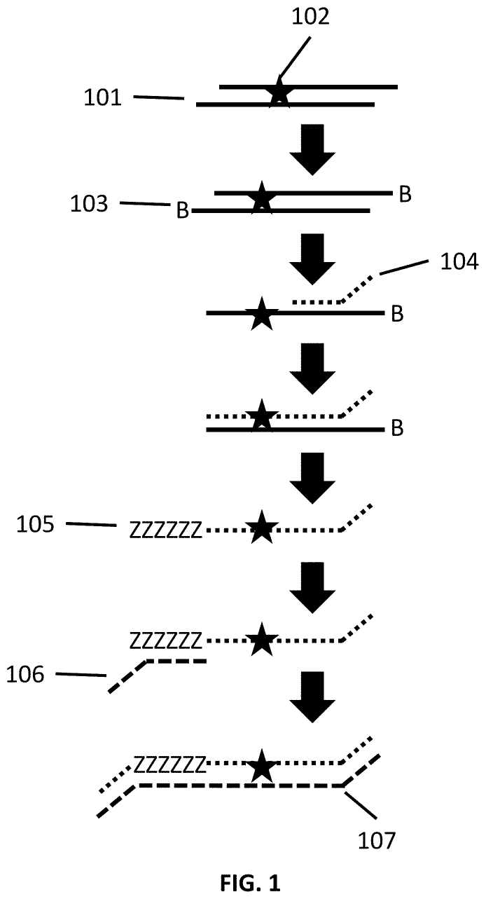

FIG. 1 illustrates an exemplary protocol that merges the library generation and enrichment to a single workflow, which can be faster and more efficient at recovering degraded DNA. First, 3′ ends of DNA molecules 101 in the extract are modified, so they are blocked 103 and will not be extended by any polymerase. Next, a sequencing adapter-tailed primer 104 is designed to bind near the site of interest 102 (most often a SNP, but could be miniSTR or other site), and is extended past the site of interest to the end of the DNA fragment. After removing unused primers, a terminal transferase is added and only the extended primers are given a tail 105, since other fragments are blocked. Removal of unused primers can be conducted enzymatically (e.g., by digestion with an exonuclease) or by binding of labeled nucleotides (e.g., biotinylated nucleotides) incorporated in the extension. The tail is used to reverse prime with another adapter-containing primer 106, converting the DNA into a library 107 ready for amplification and sequencing. For higher sensitivity, a linear amplification step can be added by cycling the first extension step prior to removal of un-extended primer.

In some embodiments, a first adapter is added to the sample of nucleic acids in a first single-sided PCR reaction using a first PCR primer. Single-sided PCR uses a single primer that base pairs with and binds to a sequence in a nucleic acid, and is then extended in a templated fashion by a polymerase.

Accordingly, the disclosure provides methods of preparing a library of nucleic acids, comprising: (a) providing a sample of nucleic acids comprising at least one sequence of interest; (b) contacting the sample of nucleic acids with a plurality of first polymerase chain reaction (PCR) primers and a first polymerase under conditions that allow PCR to occur, thereby generating a plurality of first single-sided PCR products; (c) contacting the plurality of first single-sided PCR products with a terminal transferase under conditions sufficient to transfer dNTPs to the 3′ ends of the plurality of first single-sided PCR products, thereby generating a plurality of PCR products comprising 3′ tails; and (d) contacting the plurality of PCR products comprising 3′ tails, a plurality of second PCR primers and the first polymerase under conditions that allow PCR to occur; thereby generating a library of nucleic acids with adapters at the 5′ and 3′ ends.

In some embodiments, step (a) is carried out in an emulsion. As used herein, emulsion PCR refers to a method of PCR where dilution and compartmentalization of template molecules in water droplets occurs in a water-in-oil emulsion. The dilution can be controlled such that a plurality of droplets each contain a single template molecule, and function as micro PCR reaction vessels. Emulsion PCR can prevent the loss of rare templates through non-amplification or competition with more abundant templates.

In some embodiments, the first PCR primer comprises (i) a sequence complementary to a sequence adjacent to or overlapping the at least one sequence of interest, and (ii) a first adapter sequence. In some embodiments, the first adapter sequence is 5′ of the sequence complementary to the sequence adjacent to or overlapping the at least one target sequence.

As used herein, “adjacent” refers to a sequence within 1-500, 1-300, 1-100, 1-75, 1-50 or 1-25 nucleotides of another sequence, for example a sequence of interest. Sequences that are “overlapping” can be wholly, or partly overlapping. For example, sequences that overlap by 1, 2, 3, 4, 5, 6, 7, 8, 9, 10, 11, 12, 13, 14, 15, 16, 17, 18, 19, 20, 21, 22, 23 24, 25 or more nucleotides are considered to be overlapping. In exemplary embodiments, the sequence of interest comprises a forensically interesting SNP, and the first PCR primer binds within 1-20 nucleotides of the SNP.

In some embodiments, the sequence complementary to a sequence adjacent to or overlapping the at least one sequence of interest is a known sequence. For example, the sequence of interest is a SNP, miniSTR or other known polymorphism, and the sequence of adjacent or overlapping sequences are known in the art. For example, the SNP or miniSTR may be in a genome of a species whose genome has been sequenced, such as the human genome. Many genome sequences are publicly available, for example at https://www.ncbi.nlm.nih.gov/genome/.

In some embodiments, the methods described herein comprise non-targeted, or random, primer extension-based sequencing methods using single-sided PCR. Non-targeted methods can be used to add adapters to a plurality of sequences of interest. Alternatively, or in addition, non-targeted methods can be used add adapters to sequences when the sequence of interest is not known. In some embodiments, the methods described herein comprise a PCR primer with a random priming sequence.

Random priming sequences can be used to add either a first or a second adapter to the samples of nucleic acids described herein. For example, a random priming sequence on a first PCR primer may be used to add first adapter to a sample of nucleic acids in a first single-sided PCR reaction, followed a 3′ tailing reaction, followed by a second single-sided PCR reaction comprising a second PCR primer comprising a sequence of a second adapter and a sequence complementary to the 3′ tails of the nucleic acids in the sample. Alternatively, the nucleic acids in the sample may first be 3′ tailed, followed by a first single-sided PCR reaction with a first PCR primer comprising a first adapter and a sequence complementary to the 3′ tails, followed a second single-sided PCR reaction comprising a second PCR primer comprising a second adapter and a random priming sequence.

In some embodiments of the first PCR primer, the sequence complementary to a sequence adjacent to or overlapping the at least one sequence of interest is a random sequence. Random sequences may be any suitable length. In some embodiments, the random sequence comprises 3, 4, 5, 6, 7, 8, 9, 10, 11, 12, 13, 14, 15, 16, 17, 18, 19 or 20 random nucleotides. In some embodiments, the random sequence is a random 4mer, 6mer, 8mer, 9mer, 10mer, 11mer or 12mer. In some embodiments, the random sequence is a random 9mer.

In some embodiments of the first PCR primer, the sequence complementary to a sequence adjacent to or overlapping the at least one sequence of interest is not a random sequence.

In some embodiments, untemplated dNTPs are added to the 3′ end of the plurality of first single-sided PCR products. The untemplated dNTPs can be dATPs (a polyA tail), dCTPs (a polyC tail), dGTPs (a polyG tail) or dTTPs (a polyT tail). In some embodiments, the untemplated 3′ nucleotides are polyGs (G-tailing). G-tailing can provide superior consistency to A-tailing across a variety of sample DNA input concentrations.

Untemplated nucleotides can be added to nucleic acid samples using a terminal transferase. Exemplary terminal transferases include Terminal Transferase (TdT) from NEB.

In exemplary embodiments, 1:1000 pmol 3′ DNA ends to pmol dNTPs are used for the tailing reaction.

In exemplary embodiments, 1:2500 pmol ends to pmol dNTPs are used for the tailing reaction.

In exemplary embodiments, between 1:1000 pmol ends to pmol dNTPs and 1:50,000 pmol ends to pmol dNTPs are used for the tailing reaction.

In exemplary embodiments, 1:1000 pmol ends to pmol dNTPs, 1500 pmol ends to pmol dNTPs, 1:2000 pmol ends to pmol dNTPs, 1:2500 pmol ends to pmol dNTPs, 1:3000 pmol ends to pmol dNTPs, 1:3500 pmol ends to pmol dNTPs, 1:4000 pmol ends to pmol dNTPs, 1:4500 pmol ends to pmol dNTPs, 1:5000 pmol ends to pmol dNTPs, 1:5500 pmol ends to pmol dNTPs, 1:6000 pmol ends to pmol dNTPs, 1:6500 pmol ends to pmol dNTPs, 1:7000 pmol ends to pmol dNTPs, 1:7500 pmol ends to pmol dNTPs, 1:8000 pmol ends to pmol dNTPs, 1:9000 pmol ends to pmol dNTPs, 1:10,000 pmol ends to pmol dNTPs, 1:20,000 pmol ends to pmol dNTPs, 1:25,000 pmol ends to pmol dNTPs, 1:30,000 pmol ends to pmol dNTPs, 1:35,000 pmol ends to pmol dNTPs, 1:40,000 pmol ends to pmol dNTPs, 1:45,000 pmol ends to pmol dNTPs or 1:50,000 pmol ends to pmol dNTPs are used for the tailing reaction. In some embodiments, the dNTPs are dATPs, DCTPs, DGTPs or dTTPs. In some embodiments, the dNTPs are dGTPs.

Higher concentrations of dNTPs in the tailing reaction lead to longer tails on the nucleic acid molecules in the sample following tailing. Without wishing to be bound by theory, it is thought that longer tails may improve library performance, as these longer tails allow for a higher chance that the complementary sequence used to attach the adapter will find and hybridize with the tail.

In exemplary embodiments, 0.2 U/μL Terminal transferase up to 5 pmol are used. In exemplary embodiments, the terminal transferase reactions are incubated at 37° C. for 30 minutes, and then at 70° C. for 10 minutes.

In some embodiments, the tailed single-sided PCR product is purified following tailing using the purification methods described herein.

In some embodiments, a second adapter is added to the sample of nucleic acids in a second single-sided PCR reaction following 3′ tailing.

In some embodiments, the second PCR primer for the second PCR reaction comprises (i) a sequence complementary to the 3′ tails added to first PCR products at the tailing step, and (ii) a second adapter sequence. For example, if the tailing step added polyG tails to the nucleic acids in the sample, the second PCR primer comprises a polyC sequence to facilitate base-pairing with the polyG tails. In some embodiments, the second adapter sequence is 5′ of the sequence complementary to the 3′ tail. In exemplary embodiments, the tailing step comprises adding polyG tails to the plurality of first single-sided PCR products, and the sequence complementary to the polyG tails comprises 3, 4, 5, 6, 7, 8, 9, 10, 11, 12, 13, 14, 15, 16, 17, 18, 19 or 20 Cs. In some embodiments, the sequence complementary to the polyG tails comprises a sequence of CCCCCCC.

In some embodiments, the second single-sided PCR product is purified following the second single-sided PCR reaction using the methods described herein.

In some embodiments, the sample of nucleic acids are 3′ tailed before addition of the first and second adapters.

Accordingly, the disclosure provides methods of preparing a library of nucleic acids, comprising: (a) providing a sample of nucleic acids comprising at least one sequence of interest; (b) contacting the sample of nucleic acids with a terminal transferase and dNTPs under conditions sufficient to transfer dNTPs to the 3′ ends of the nucleic acids, thereby generating a sample of nucleic acids comprising 3′ tails; (c) contacting the sample of nucleic acids comprising 3′ tails with a plurality of first polymerase chain reaction (PCR) primers, and a first polymerase under conditions that allow PCR to occur, thereby generating a plurality of first single-sided PCR products; and (d) contacting the plurality of first single-sided PCR products with a plurality of second PCR primers, and the first polymerase under conditions that allow PCR to occur; thereby generating a library of nucleic acids with adapters at the 5′ and 3′ ends.

In some embodiments, the 3′ tailing at step (b) is carried out in an emulsion.

In some embodiments, the single-sided PCR reaction at step (c) is carried out in an emulsion.

In some embodiments, untemplated dNTPs are added to the 3′ ends of a plurality of nucleic acids in the sample. The untemplated dNTPs can be dATPs (a polyA tail), dCTPs (a polyC tail), dGTPs (a polyG tail) or dTTPs (a polyT tail). In some embodiments, the untemplated 3′ nucleotides are polyGs (G-tailing). G-tailing can provide superior consistency to A-tailing across a variety of sample DNA input concentrations.

In some embodiments, the plurality of first PCR primers comprise (i) a sequence complementary to the 3′ tails from step (c), and (ii) a first adapter sequence. In some embodiments, the first adapter sequence if 5′ of the sequence complementary to the 3′ tail. In exemplary embodiments, the tailing step comprises adding polyG tails to the plurality of first single-sided PCR products, and the sequence complementary to the polyG tails comprises 3, 4, 5, 6, 7, 8, 9, 10, 11, 12, 13, 14, 15, 16, 17, 18, 19 or 20 Cs. In some embodiments, the sequence complementary to the polyG tails comprises a sequence of CCCCCCC.

In some embodiments, the plurality of second PCR primers comprise (i) a sequence complementary to a sequence adjacent to or overlapping the at least one sequence of interest, and (ii) a second adapter sequence. In some embodiments, the second adapter sequence is 5′ of the sequence complementary to the sequence adjacent to or overlapping the at least one sequence of interest.

In some embodiments of the second PCR primer, the sequence complementary to a sequence adjacent to or overlapping the at least one sequence of interest is a random sequence. Random sequences may be any suitable length. In some embodiments, the random sequence comprises 3, 4, 5, 6, 7, 8, 9, 10, 11, 12, 13, 14, 15, 16, 17, 18, 19 or 20 random nucleotides. In some embodiments, the random sequence is a random 4mer, 6mer, 8mer, 9mer, 10mer, 11 mer or 12mer. In some embodiments, the random sequence is a random 9mer.

In some embodiments of the second PCR primer, the sequence complementary to a sequence adjacent to or overlapping the at least one sequence of interest is not a random sequence.

In some embodiments, the first polymerase is a Klenow Fragment. In some embodiments, the first polymerase is a Taq polymerase. In some embodiments, the first polymerase is a high-fidelity polymerase, for example a Qiagen high fidelity polymerase. In some embodiments, the first polymerase is a Phi29 polymerase.

In some embodiments, the first polymerase is suitable for isothermal amplification reactions. Unlike conventional PCR, isothermal amplification is carried out a constant temperature, and does not require a thermocycler. Exemplary polymerases suitable for isothermal amplification include, but are not limited to, Phi29, Klenow exo- or Bsu DNA Polymerase, Large Fragment.

In some embodiments, the first and/or second single-sided PCR reaction that adds the first and/or second adapter is an isothermal PCR reaction. In some embodiments, the polymerase is Phi29.

In specific embodiments, the polymerase is Phi29, and the single-sided PCR reaction comprises incubation at 30° C. for 60 minutes, followed by incubation at 65° C. for 10 minutes.

Additional suitable polymerases will be known to persons of ordinary skill in the art.

The sample of nucleic acids may optionally be purified between any of the steps of library preparation described herein. The sample of nucleic acids may be purified after 3′ tailing, after the first single-sided PCR reaction, or after the second single-sided PCR reaction, or any combination thereof. Purification can include removal of unincorporated nucleotides (e.g. ddNTPs or dNTPs) introduced in any of the previous reactions. Reaction products can be purified enzymatically, for example by using alkaline phosphatase, or using a bead or column-based purification strategy. In some embodiments, the alkaline phosphatase is recombinant shrimp alkaline phosphatase. An exemplary column purification strategy comprises the MinElute PCR purification kit, although alternative purification strategies will be known to persons of ordinary skill in the art. Exemplary bead-based purification strategies include solid phase reversible immobilization (SPRI) magnetic beads. SPRI beads are available commercially, for example from Ampure or abm Inc.

In specific embodiments, the methods comprise purifying the 3′ tailed, first single-sided PCR reaction, and/or second single-sided PCR reaction products with SPRI beads at a 1.8× dilution, and eluting in 10 to 30 μL of a suitable buffer or water.

### Sample Preparation

The disclosure provides methods of preparing a sample nucleic acids for generating a library using the methods of library preparation described herein, such as library preparation through the use of single-sided PCR.

In some embodiments, the methods comprise blunting overhangs of the nucleic acids in the sample prior to the first single-sided PCR reaction. The overhangs can be 5′ or 3′ overhangs, and the nucleic acids comprise double stranded DNA. Blunting is a process in which single-stranded overhangs created by restriction digest or shearing are filled in by addition of nucleotides to the complementary strand, or by removing the overhang with an exonuclease. Exemplary blunting enzymes include T4 polymerase, Klenow fragment or Mung Bean Nuclease. For example, 1 Unit (U) T4 DNA polymerase per μg of sample DNA can be used. Blunting allows for the efficient incorporation of dNTPs or ddNTPs at the ends of DNAs by enzymes such as the Klenow fragment. In some embodiments, the methods comprise contacting the sample of nucleic acids with a first enzyme prior to 3′ tailing or the first single-sided PCR reaction, depending on the order of adapter addition, under conditions that allow for blunting of overhangs in the sample of nucleic acids, thereby generating a sample of blunt ended nucleic acids. The first enzyme can be T4 polymerase, Klenow fragment or Mung Bean Nuclease.

In some embodiments, the blunted sample of nucleic acids is purified following blunting. Purification can include removal of unincorporated nucleotides (e.g. dNTPs) introduced in the blunting reaction. The blunted sample of nucleic acids can be purified enzymatically, for example by using recombinant shrimp alkaline phosphatase, or using a bead or column-based purification strategy. An exemplary column purification strategy comprises the Qiaquick PCR purification kit, although alternative purification strategies will be known to the person of ordinary skill in the art.

In exemplary embodiments, 1 Unit (U) T4 DNA polymerase per μg DNA is used to blunt the sample of nucleic acids. In an exemplary embodiment, the reaction is incubated at 12° C. for 15 minutes, and then at 75° C. for 20 minutes.

In some embodiments, the methods comprising blocking the 3′ ends blunted sample of nucleic acids. Blocking can be accomplished by using a second enzyme to incorporate dideoxynucleotides (ddNTPs) at the 3′ ends of blunted DNAs. In some embodiments, the enzyme is the Klenow fragment. The Klenow fragment is a fragment of DNA polymerase I that retains 5′ to 3′ polymerase activity and 3′ to 5′ exonuclease activity, but does not have 5′ to 3′ exonuclease activity.

In an exemplary embodiment, the sample of nucleic acids is incubated with Klenow, ddNTPs and a suitable buffer for 40 minutes at 37° C., and then for 75° C. for 20 minutes.

In some embodiments, the blocked sample of nucleic acids is purified following blocking. Purification can include removal of unincorporated nucleotides (e.g. ddNTPs) introduced in the blocking reaction. The blocked sample of nucleic acids can be purified enzymatically, for example by using alkaline phosphatase, or using a bead or column-based purification strategy. In some embodiments, the alkaline phosphatase is recombinant shrimp alkaline phosphatase. An exemplary column purification strategy comprises the Qiaquick Nucleotide removal kit, although alternative purification strategies will be known to persons of ordinary skill in the art.

### Ligation-Free Preparation of Nucleic Acids by Strand Switching

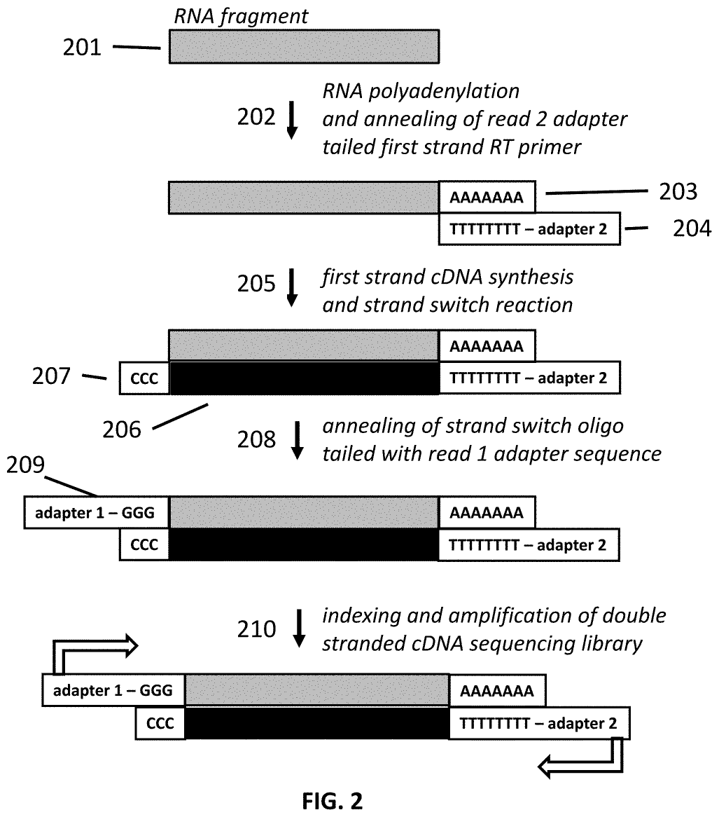

RNA can be prepared for sequencing (e.g., as cDNA) using a strand-switching method. FIG. 2 shows an exemplary schematic of such a strand-switching method. RNA molecules 201 can be polyadenylated 202 or otherwise given a tail (e.g., a poly-A/polyA/po tail) 203. An oligonucleotide comprising an adapter (here, “Adapter 2”) 204 can be hybridized to the RNA tail, for example via a poly-T/polyT region of the oligonucleotide. Reverse transcription 205 can then be used to synthesize cDNA 206. A region such as a poly-C/polyC region 207 can be added to the cDNA for example by using MMLV as the reverse transcriptase, which can enable strand-switching. A strand-switching oligonucleotide 209 can then be hybridized to the cDNA tail (e.g., the poly-C tail), for example via a poly-G/polyG region of the oligonucleotide. The strand-switching oligonucleotide can comprise an adapter (here, “Adapter 1”). The adapters can then be used for amplification and/or indexing 210 of a double stranded cDNA sequencing library.

The adapters can comprise sequencing adapters (e.g., Illumina sequencing adapters). The adapters can comprise unique molecular identifier (UMI) sequences. The UMI sequences can comprise a sequence that is unique to each original RNA molecule (e.g., a random sequence). In some embodiments, the UMI comprises 2, 3, 4, 5, 6, 7, 8, 9, 10, 11, or 12 nucleotides. In some embodiments, the UMI is more than 12 nucleotides. In some embodiments, the UMI comprises or consists essentially of a random sequence. This can allow quantification of RNA amounts, free from sequencing bias. The adapters can comprise “barcode” sequences. The barcode sequences can comprise a barcode sequence that is shared among RNA molecules from a particular source (such as a subject, patient, environmental sample, partition (e.g., droplet, well, bead)). This can allow pooling of sequencing information for subsequent analysis, and can allow detection and elimination of cross-contamination. The adapters can comprise multiple distinct sequences, such as a UMI unique to each RNA molecule, a barcode shared among RNA molecules from a particular source, and a sequencing adapter.

The cDNA library can be further processed according to methods of the present disclosure, such as by targeted digestion or other depletion. For example, cDNA from a host (e.g., a human) can be digested or otherwise depleted, while cDNA from a non-host (e.g., an infectious agent) can remain. The cDNA can be sequenced or otherwise analyzed (e.g., hybridization assay, amplification assay).

### PCR Primers and Adapters

Provided herein are PCR primers for single-sided PCR reactions to produce the ligation-free libraries described herein.

In some embodiments, the first PCR primer comprises (i) a sequence complementary to a sequence adjacent to or overlapping the at least one sequence of interest, and (ii) a first adapter sequence. In some embodiments, the first adapter sequence is 5′ of the sequence complementary to the sequence adjacent to or overlapping the at least one sequence of interest.

In some embodiments, the sequence complementary to a sequence adjacent to or overlapping the at least one sequence of interest is a not a random sequence.

In some embodiments, the sequence complementary to a sequence adjacent to or overlapping the at least one sequence of interest is a random sequence.

Random sequences may be any suitable length. In some embodiments, the random sequence comprises 3, 4, 5, 6, 7, 8, 9, 10, 11, 12, 13, 14, 15, 16, 17, 18, 19 or 20 random nucleotides. In some embodiments, the random sequence is a random 4mer, 6mer, 8mer, 9mer, 10mer, 11mer or 12mer. In some embodiments, the random sequence is a random 9mer.

In exemplary embodiments, the first PCR primer comprises a sequence of 5′-ACACTCTTTCCCTACACGACGCTCTTCCGATCNNNNNNNNN-3′ (SEQ ID NO:1).

In some embodiments, the first PCR primer comprises (i) a sequence complementary to a 3′ tail added to the nuclei acids in the sample by a terminal transferase, and (ii) a first adapter sequence. In some embodiments, the first adapter sequence is 5′ of the sequence complementary to the sequence adjacent to or overlapping the at least one sequence of interest.

In some embodiments, the 3′ tail comprises a polyA, polyC, polyT or polyG tail.

In specific embodiments, the 3′ tail comprises a 3′ a polyG tail, the sequence complementary to the polyG tails comprises 3, 4, 5, 6, 7, 8, 9, 10, 11, 12, 13, 14, 15, 16, 17, 18, 19 or 20 Cs. In some embodiments, the sequence complementary to the polyG tails comprises a sequence of CCCCCCC.

In exemplary embodiments, the first PCR primer comprises a sequence of GACTGGAGTTCAGACGTGTGCTCTTCCGATCCCCCCC-3′ (SEQ ID NO:2).

In some embodiments, the first adapter comprises a sequence of GACTGGAGTTCAGACGTGTGCTCTTCCGAT (SEQ ID NO:3).

In some embodiments, the first adapter comprises a sequence of ACACTCTTTCCCTACACGACGCTCTTCCGATC (SEQ ID NO:4).

In some embodiments, the first adapter comprises a sequence of GACTGGAGTTCAGACGTGTGCTCTTCCGATCT (SQE ID NO:3) and the second adapter comprises a sequence of ACACTCTTTCCCTACACGACGCTCTTCCGATCT (SEQ ID NO:4). In some embodiments, the first adapter comprises a sequence of ACACTCTTTCCCTACACGACGCTCTTCCGATCT (SEQ ID NO:4) and the second adapter comprises a sequence of GACTGGAGTTCAGACGTGTGCTCTTCCGATCT (SEQ ID NO:3).

In some embodiments, the second PCR primer comprises (i) a sequence complementary to a sequence adjacent to or overlapping the at least one sequence of interest, and (ii) a second adapter sequence. In some embodiments, the second adapter sequence is 5′ of the sequence complementary to the sequence adjacent to or overlapping the at least one sequence of interest.

In some embodiments, the sequence complementary to a sequence adjacent to or overlapping the at least one sequence of interest is a not a random sequence.

In some embodiments, the sequence complementary to a sequence adjacent to or overlapping the at least one sequence of interest is a random sequence.

Random sequences may be any suitable length. In some embodiments, the random sequence comprises 3, 4, 5, 6, 7, 8, 9, 10, 11, 12, 13, 14, 15, 16, 17, 18, 19 or 20 random nucleotides. In some embodiments, the random sequence is a random 4mer, 6mer, 8mer, 9mer, 10mer, 11 mer or 12mer. In some embodiments, the random sequence is a random 9mer.

In exemplary embodiments, the second PCR primer comprises a sequence of 5′-ACACTCTTTCCCTACACGACGCTCTTCCGATCNNNNNNNNN-3′ (SEQ ID NO:5).

In some embodiments, the second PCR primer comprises (i) a sequence complementary to a 3′ tail added to the nuclei acids in the sample by a terminal transferase, and (ii) a second adapter sequence. In some embodiments, the second adapter sequence is 5′ of the sequence complementary to the sequence adjacent to or overlapping the at least one sequence of interest.

In some embodiments, the 3′ tail comprises a polyA, polyC, polyT or polyG tail.

In specific embodiments, the 3′ tail comprises a 3′ a polyG tail, the sequence complementary to the polyG tails comprises 3, 4, 5, 6, 7, 8, 9, 10, 11, 12, 13, 14, 15, 16, 17, 18, 19 or 20 Cs. In some embodiments, the sequence complementary to the polyG tails comprises a sequence of CCCCCCC.

In exemplary embodiments, the second PCR primer comprises a sequence of GACTGGAGTTCAGACGTGTGCTCTTCCGATCCCCCCC-3′ (SEQ ID NO:2).

In some embodiments, the second adapter comprises a sequence of GACTGGAGTTCAGACGTGTGCTCTTCCGATCT (SEQ ID NO:3).

In some embodiments, the second adapter comprises a sequence of ACACTCTTTCCCTACACGACGCTCTTCCGATCT (SEQ ID NO:4).

In some embodiments, the first adapter comprises a first unique molecular identifier (UMI). In some embodiments, the first UMI comprises 2, 3, 4, 5, 6, 7, 8, 9, 10, 11, or 12 nucleotides. In some embodiments, the first UMI is more than 12 nucleotides. In some embodiments, the first UMI comprises or consists essentially of a random sequence.

In some embodiments, the first adapter comprises a sequencing adapter, for example for Illumina sequencing.

In some embodiments, the second adapter comprises a first unique molecular identifier (UMI). In some embodiments, the second UMI comprises 2, 3, 4, 5, 6, 7, 8, 9, 10, 11, or 12 nucleotides. In some embodiments, the second UMI is more than 12 nucleotides. In some embodiments, the second UMI comprises or consists essentially of a random sequence.

In some embodiments, the second adapter comprises a sequencing adapter, for example for Illumina sequencing.

In some embodiments, the first and/or second adapter comprises a sequence of a NEBNext Adapter. The ordinarily skilled artisan will be able to design adapters suited to particular high-throughput sequencing platforms and applications.

Adapters can be 10 to 100 bp in length although adapters outside of this range are usable without deviating from the present disclosure.

An adapter may be configured for any next generation sequencing platform, for example for use on an Illumina sequencing platform such as HiSeq or MiSeq, or for use on an IonTorrent platform, or for use with Nanopore technology.

In some embodiments, the adapters comprise multiple distinct sequences, such as a UMI unique to each nucleic acid molecule, a barcode shared among nucleic acid molecules from a particular source, and a sequencing adapter.

In some embodiments, the first PCR primer, the second PCR primer, or both comprise at least one phosphorothioate linkage. Phosphorothioate linkages, or bonds substitute a sulfur atom for a non-bridging oxygen in the phosphate backbone of a polynucleotide. This modification renders the internucleotide linkage resistant to nuclease degradation, such as degradation by Phi29 polymerase. Phosphorothioate bonds can be introduced between the last 3-5 nucleotides at the 5′- or 3′-end of the oligo to inhibit exonuclease degradation. In some embodiments, the first PCR primer, the second PCR primer, or both comprise two 3′ phosphorothioate linkages. In exemplary embodiments, the first PCR primer comprises a sequence of 5′-A*C*ACTCTTTCCCTACACGACGCTCTTCCGATCNNNNNNN*N*N-3′, where (*) represent phosphorothioate linkages. In exemplary embodiments, the second PCR primer comprises a sequence of G*A*CTGGAGTTCAGACGTGTGCTCTTCCGATCCCCC*C*C- where (*) represent phosphorothioate linkages.

In some embodiments, the first and/or second adapter comprises a spacer sequence. Spacer sequence can minimize non-template PCR products. In some embodiments, the spacer comprises 1, 2, 3, 4, 5, 6, 7, 8, 9, 10, 11, 12, 13, 14, 15, 16, 17, 18, 19 or 20 nucleotides. In some embodiments, the spacer is 3 nucleotides. In some embodiments, the spacer comprises a polyC sequence. In some embodiments, the spacer comprises a sequence of CCC. In some embodiments, the spacer is 5′ of the adapter sequence.

### Indexing

Provided herein are methods of indexing libraries of nucleic acids, for example for high throughput sequencing. Indexing adds unique identifiers to sequences during sample preparation, to allow the sequencing of multiple pooled samples in a single sequencing run.

In some embodiments, indexing sequences are added to the second single-sided PCR product in an indexing PCR reaction. For example, in those embodiments where the first and second adapters do not comprise UMI sequences, indexing sequences comprising UMI sequences, and optionally, additional adapter sequences tailored to particular high-throughput sequencing platforms can be added in an indexing PCR reaction.

Primers can also incorporate barcode or unique molecular identifier (UMI) sequences, enabling tracking of distribution of targeted sites to gain quantitative information, removal of amplification errors, and prevention of cross-contamination from other samples. For example, with two flanking 8-mer UMIs more than 4 billion combinations (4¹⁶) per primer are possible. As an additional metric, in some applications of the methods, for example, those involving restriction digest prior to library preparation, the 3′ breakpoint for the original molecule is known, making it virtually impossible to encounter the same combination multiple times. With a database of previously used UMIs for each primer, contamination from previously handled samples can be monitored. Importantly, these data can be stored without keeping identifiable information to protect privacy.

In some embodiments, the second adapter comprises a second unique molecular identifier (UMI). In some embodiments, the second UMI comprises 2, 3, 4, 5, 6, 7, 8, 9, 10, 11, or 12 nucleotides. In some embodiments, the second UMI is more than 12 nucleotides. In some embodiments, the second UMI comprises or consists essentially of a random sequence. In some embodiments, the first and second UMI sequences are the same sequence. In some embodiments, the first and second UMI sequences are not the same sequence.

In some embodiments, the second adapter comprises a sequencing adapter, for example for Illumina sequencing.

In some embodiments, the second adapter comprises a sequence of a NEBNext Adapter. The ordinarily skilled artisan will be able to design adapters suited to particular high-throughput sequencing platforms and applications.

In some embodiments, the methods comprise contacting the plurality of PCR products from the second single-sided PCR reaction with a plurality of first indexing primers, a plurality of second indexing primers, and a polymerase under conditions that allow PCR to occur.

In some embodiments, first indexing primer comprises a sequence complementary to the first adapter and a first unique molecular identifier sequence (UMI). For example, if the first adapter comprises a sequence of a NEBNext adapter, the indexing primer comprises a sequence complementary to the NEBNext adapter sequence of the first adapter. In some embodiments, the first UMI sequence is 5′ of the sequence complementary to the first adapter. In some embodiments, the first UMI comprises 2, 3, 4, 5, 6, 7, 8, 9, 10, 11, or 12 nucleotides. In some embodiments, the first UMI is more than 12 nucleotides. In some embodiments, the first UMI comprises or consists essentially of a random sequence. In some embodiments, the first indexing primer comprises a sequencing adapter, for example for Illumina sequencing.

In some embodiments, the second indexing primer comprises a sequence complementary to the second adapter and a second UMI sequence. For example, if the second adapter comprises a sequence of a second NEBNext adapter, the second indexing primer comprises a sequence complementary to the second NEBNext adapter sequence of the second adapter. In some embodiments, the second UMI sequence is 5′ of the sequence complementary to the second adapter. In some embodiments, the second UMI comprises 2, 3, 4, 5, 6, 7, 8, 9, 10, 11, or 12 nucleotides. In some embodiments, the second UMI is more than 12 nucleotides. In some embodiments, the second UMI comprises or consists essentially of a random sequence. In some embodiments, the first and second UMI sequences are the same sequence. In some embodiments, the first and second UMI sequences are not the same sequence.

In some embodiments, the second indexing primer comprises a sequencing adapter, for example for Illumina sequencing. The ordinarily skilled artisan will be able to design indexing primers suited to particular high-throughput sequencing applications.

In an embodiment, the indexing PCR reaction comprises 6 polymerase extension cycles. The number of polymerase extension cycles can be calculated based off of qPCR plateau values quantifying the amount of PCR product from the second single-sided PCR reaction.

In some embodiments, the indexing PCR product is purified following indexing PCR. In some embodiments, the purification comprises Kapa Pure beads (Roche).

### Analysis

High-throughput sequencing data generated using the methods described herein can be analyzed using any methods known in the art. Software tools for analyzing high-throughput sequencing data include, but are not limited to, Samtools, FastQC, BWA, GenomeMapper, Novoalign, mrsFAST, Bowtie, GEM mapper, MoDIL, BreakDancer, Splitread, DeNovoGear and Scalpel.

Sites of interest can be used to determine identity of a subject. In some cases, identity can be determined using identity by state (IBS) or identity-by-decent (IBD). In identifying different genealogical relationships, relationship can be defined as R (k₀, k₁, k₂), where kₘ matches the fraction of the genome where the two individuals share m alleles. Table 1 has expected values for relationships typically relevant in forensics. This can be formulated in Bayesian terms as: R=((IBD=k₀|Data), (IBD=k₁|Data, P(IBD=k₂|Data). Combining this with the expected values from table 1, we can setup a likelihood ratio test as:

$$LR = \frac{L ( H ( Data ) )}{L ( H ( Expected ) )} = {\prod }_{i = 0}^{2} \frac{P ( IBD = {k}_{i} \vert Data )}{P ( IBD = {k}_{i} \vert Expected )}$$

A measure of significance is the obtained by making use of the following asymptotic property:

−2 log(LR)˜χ_{d}²

where dis degrees of freedom.

TABLE 1
Expected allele sharing among related individuals.

| Relationship | k0 | k1 | k2 |
|---|---|---|---|
| Self/mono-zygotic twin | 0 | 0 | 1 |
| Parent-Offspring | 0 | 1 | 0 |
| Full Siblings | 0.25 | 0.5 | 0.25 |
| Niece, nephew, uncle, aunt, | 0.5 | 0.5 | 0 |
| grandparent, grandchild, | | | |
| half-sibling | | | |
| First cousins | 0.75 | 0.25 | 0 |
| Unrelated | 1 | 0 | 0 |

High-throughput sequencing can enable analysis of a huge pool of degraded/trace forensics samples that are refractory to current STR-based genotyping methods. The SNP data generated by HTS also contains information that STR profiles do not, including ancestry and phenotype predictions that can be used to generate investigative leads. As such, the methods disclosed herein can serve as a supplement for samples where partial or no CODIS profile can be generated, and can add additional data for investigative leads in cases where no match is found in the CODIS database. However, for the forensics community to transition to HTS, it needs the tools to collect and analyze SNP data in the most efficient, inexpensive, and targeted way possible. The methods disclosed herein can give a reliable way of testing highly degraded samples, by focusing extraction methods on shorter DNA fragments and targeting sequencing to sites of interest, followed by analysis with a streamlined informatics pipeline backed by strong statistical analyses.

### Sequences of Interest

Provided herein are methods of libraries from nucleic acid samples comprising a sequence of interest, and methods of enriching libraries for a sequence of interest through depletion of targeted sequences.

Nucleic acid samples can include ribonucleic acid samples (RNA), deoxyribonucleic acid samples (DNA) or samples comprising a mixture of DNA and RNA.

In some embodiments, the sequences of interest are genomic sequences (genomic DNA). In some embodiments, the sequences of interest are mammalian genomic sequences. In some embodiments, the sequences of interest are eukaryotic genomic sequences. In some embodiments, the sequences of interest are prokaryotic genomic sequences. In some embodiments, the sequences of interest are viral genomic sequences. In some embodiments, the sequences of interest are bacterial genomic sequences. In some embodiments, the sequences of interest are plant genomic sequences. In some embodiments, the sequences of interest are microbial genomic sequences. In some embodiments, the sequences of interest are genomic sequences from a parasite, for example a eukaryotic parasite. In some embodiments, the sequences of interest are host genomic sequences (e.g., the host organism of a microbiome, a parasite, or a pathogen). In some embodiments, the sequences of interest are abundant genomic sequences, such as sequences from the genome or genomes of the most abundant species in a sample.

In some embodiments, the sequences of interest comprise repetitive DNA. In some embodiments, the sequences of interest comprise abundant DNA. In some embodiments, the sequences of interest comprise mitochondrial DNA. In some embodiments, the sequences of interest comprise ribosomal DNA. In some embodiments, the sequences of interest comprise centromeric DNA. In some embodiments, the sequences of interest comprise DNA comprising Alu elements (Alu DNA). In some embodiments, the sequences of interest comprise long interspersed nuclear elements (LINE DNA). In some embodiments, the sequences of interest comprise short interspersed nuclear elements (SINE DNA). In some embodiments, the abundant DNA comprises ribosomal DNA.

In some embodiments, the sequences of interest comprise single nucleotide polymorphisms (SNPs), short tandem repeats (STRs), cancer genes, inserts, deletions, structural variations, exons, genetic mutations, or regulatory regions.

In some embodiments, the sequences of interest can be a genomic fragment, comprising a region of the genome, or the whole genome itself. In one embodiment, the genome is a DNA genome. In another embodiment, the genome is an RNA genome.

In some embodiments, the sequences of interest are from a eukaryotic or prokaryotic organism; from a mammalian organism or a non-mammalian organism; from an animal or a plant; from a bacteria or virus; from an animal parasite; from a pathogen.

In some embodiments, the sequences of interest are from any mammalian organism. In one embodiment the mammal is a human. In another embodiment the mammal is a livestock animal, for example a horse, a sheep, a cow, a pig, or a donkey. In another embodiment, a mammalian organism is a domestic pet, for example a cat, a dog, a gerbil, a mouse, a rat. In another embodiment the mammal is a type of a monkey.

In some embodiments, the sequences of interest are from any bird or avian organism. An avian organism includes but is not limited to chicken, turkey, duck and goose.

In some embodiments, the sequences of interest are from an insect. Insects include, but are not limited to honeybees, solitary bees, ants, flies, wasps or mosquitoes.

In some embodiments, the sequences of interest are from a plant. In one embodiment, the plant is rice, maize, wheat, rose, grape, coffee, fruit, tomato, potato, or cotton.

In some embodiments, the sequences of interest are from a species of bacteria. In one embodiment, the bacteria are tuberculosis-causing bacteria.

In some embodiments, the sequences of interest are from a virus.

In some embodiments, the sequences of interest are from a species of fungi.

In some embodiments, the sequences of interest are from a species of algae.

In some embodiments, the sequences of interest are from any mammalian parasite.

In some embodiments, the sequences of interest are obtained from any mammalian parasite. In one embodiment, the parasite is a worm. In another embodiment, the parasite is a malaria-causing parasite. In another embodiment, the parasite is a Leishmaniosis-causing parasite. In another embodiment, the parasite is an amoeba.

In some embodiments, the sequences of interest are from a pathogen.

In some embodiments, the sequences of interest are human sequences. In some embodiments, the human sequences are polymorphic sequences that can be used to identify individual subjects in a human population, for example single nucleotide polymorphisms (SNPs), miniSTRs (mini short tandem repeats), mitochondrial markers, Y chromosome markers, or taxonomic markers and the like.

In some embodiments, the sequence of interest comprises a disease trait marker.

In some embodiments, the sequences of interest comprise single nucleotide polymorphisms (SNPs). In some embodiments, the SNPs are used for forensic analysis of human samples. For example, the SNPs are used characterize genetic variation between subjects.

In some embodiments, the sequence of interest comprises a miniSTR. In some embodiments, the miniSTR is used for forensic analysis of human samples. For example, the miniSTR is used to characterize genetic variation between subjects.

In some embodiments, the sequences of interest comprise RNA. In some embodiments, the sequences of interest comprise a transcriptome. In some embodiments, the sequences of interest comprise sequences of specific RNA transcripts.

In some embodiments, the sequence of interest comprises a single target sequence. In some embodiments, the comprises at least 1, 2, 3, 4, 5, 6, 7, 8, 9, 10, 15, 20, 25, 30, 40, 50, 60, 70, 80, 90, 100, 500, 1000, 10,000, 50,000, 100,000 or 200,000 unique sequences of interest.

### Methods of Depletion or Enrichment

Provided herein are methods of depleting nucleic acid libraries or samples comprising nucleic acids of sequences targeted for depletion, and enriching for nucleic acids of interest. Sequences targeted for depletion can be depleted by cleavage using a nucleic acid-guided nuclease-RNA complex, or by cleavage with a modification sensitive restriction enzyme, or a combination thereof.

### Nucleotide Modification Based Methods

Provided herein are methods of enriching a sample for target nucleic acids of interest relative to nucleic acids targeted for depletion, comprising using differences in nucleotide modification between the target nucleic acids of interest and the nucleic acids targeted for depletion.

Any type of nucleotide modification is envisaged as within the scope of the disclosure. Exemplary but non-limiting examples of nucleotide modifications of the disclosure are described below.

Nucleotide modifications used by the methods of the disclosure can occur on any nucleotide (adenine, cytosine, guanine, thymine or uracil, e.g.). These nucleotide modifications can occur on deoxyribonucleic acids (DNA) or ribonucleic acids (RNA). These nucleotide modifications can occur on double or single stranded DNA molecules, or on double or single stranded RNA molecules.

In some embodiments, the nucleotide modification comprises adenine modification or cytosine modification.

In some embodiments, the adenine modification comprises adenine methylation. In some embodiments, the adenine methylation comprises N6-methyladenine (6 mA). In some embodiments, the adenine methylation comprises Dam methylation carried out by the Deoxyadenosine methylase. In some embodiments, the adenine methylation comprises EcoKI methylation. In some embodiments, the adenine modification comprises adenine modified at N⁶ by glycine (momylation).

In some embodiments, the modification comprises cytosine modification. In some embodiments, the cytosine modification comprises 5-methylcytosine (5mC), 5-hydroxymethlcytosine (5hmC), 5-formylcytosine (5fC), 5-carboxylcytosine (5caC), 5-glucosylhydroxymethylcytosine (5ghmC) or 3-methylcytosine (3mC). In some embodiments, the cytosine methylation comprises 5-methylcytosine (5mC) or N4-methylcytosine (4mC). In some embodiments, the cytosine methylation comprises Dcm methylation, DNMT1 methylation, DNMT3A methylation or DNMT3B methylation. In some embodiments, the cytosine methylation comprises CpG methylation, CpA methylation, CpT methylation, CpC methylation or a combination thereof. In some embodiments, the cytosine methylation comprises CpG methylation. For example, CpG methylation can be used to selectively target an active region in a mammalian genome for depletion using the methods of the disclosure.

In some embodiments, the cytosine modification comprises 5-hydroxymethylcytosine (5hmC), 5-formylcytosine (5fC). 5-formylcytosine is an oxidized derivative of 5mC, 5-carboxylcytosine (5caC), 5-glucosylhydroxymethylcytosine or 3-methylcytosine.

In some embodiments of the methods of the disclosure, the methods employ at least a first modification-sensitive restriction enzyme and a second modification-sensitive restriction enzyme. In some embodiments, the first and second modification-sensitive restriction enzymes are the same. In some embodiments, the first and second modification-sensitive restriction enzymes are not the same. In some embodiment, the first or second modification-sensitive restriction enzyme is a single species of restriction enzyme (e.g., AluI, or McrBC, but not both). In some embodiments, the first or second modification-sensitive restriction enzyme is a mixture of 2 or more species of modification-sensitive restriction enzymes (e.g., a mixture of FspEI and AbaSI). In some embodiments of the methods of the disclosure, more than two different methods are combined, each using a different modification-sensitive restriction enzyme or cocktail of modification-sensitive restriction enzymes.

The term “modification-sensitive restriction enzyme”, as used herein, refers to a restriction enzyme that is sensitive to the presence of modified nucleotides within or adjacent to the recognition site for the restriction enzyme. Alternatively, or in addition, the modification-sensitive restriction enzyme can be sensitive to modified nucleotides within the recognition site itself. The modification-sensitive restriction enzyme can be sensitive to modified nucleotides that are adjacent to the recognition site, for example, within 1-50 nucleotides, 5′ or 3′ of the recognition site. Nucleotide modifications of the disclosure can be within the recognition site itself, or comprise nucleotides adjacent to the recognition site (for example, within 1-50 nucleotides, 5′ or 3′ of the recognition site, or both).

Exemplary modifications capable of blocking or reducing the activity of modification-sensitive restriction enzymes include, but are not limited to, N6-methyladenine, 5-methylcytosine (5mC), 5-hydroxymethlcytosine (5hmC), 5-formylcytosine (5fC), 5-carboxylcytosine (5caC), 5-glucosylhydroxymethylcytosine, 3-methylcytosine (3mC), N4-methylcytosine (4mC) or combinations thereof. Exemplary modifications capable of blocking modification-sensitive restriction enzymes include modifications mediated by Dam, Dcm, EcoKI, DNMT1, DNMT3A, DNMT3B and TET enzymes.

In some embodiments, the modification comprises Dam methylation. Restriction enzymes that are blocked by Dam methylation include, but are not limited to, AlwI, BcgI, BclI, BsaBI, BspDI, BspEI, BspHI, ClaI, DpnII, HphI, Hpy188I, Hpy188III, MboI, MboII, NruI, Nt.AlwI, Taqα I and XbaI.

In some embodiments, the modification comprises Dcm methylation. Restriction enzymes that are blocked by Dcm methylation include, but are not limited to, Acc65I, AlwNI, ApaI, AvaI, AvaII, BanI, BsaI, BsaHI, BslI, BsmFI, BssKI, BstXI, EaeI, Esp3I, EcoO109I, MscI, NlaIV, PflMI, PspGI, PspOMI, Sau96I, ScrFI, SexAI, SfiI, SfoI and StuI. In some embodiments, the modification comprises CpG methylation. Restriction enzymes that are blocked by CpG methylation include, but are not limited to, AatII, AccII, AciI, AcII, AfeI, AgeI, Aor13HI, Aor51HI, AscI, AsiSI, AluI, AvaI, BceAI, BmgBI, BsaI, BsaHI, BsiEI, BsiWI, BsmBI, BspDI, BspT104I BsrFalphaI, BssHII, BstBI, BstUI, Cfr10I, ClaI, CpoI, EagI, Esp3I, Eco52I, FauI, FseI, FspI, HaeII, HgaI, HhaI, HpaII, HpyCH4IV, Hpy99I, KasI, MluI, NaeI, NgoMIV, NotI, NruI, Nt.BsmAI, Nt.CviPII, NsbI, PmaCI, Psp1406I, PluTI, PmlI, PvuI, RsrII SacII, SalI, SmaI, SnaBI, SfoI, SgrAI, SmaI, SrfI, Sau3AI, TspMI and ZraI.

In some embodiments, a modification-sensitive restriction enzyme is active at a recognition site comprising at least one modified nucleotide and is not active at a recognition site that does not comprise at least one modified nucleotide. Exemplary modifications recognized by modification-sensitive restriction enzymes that cleave at recognition sites comprising one or more modified nucleotides include, but are not limited to, N⁶-methyladenine, 5-methylcytosine (5mC), 5-hydroxymethlcytosine (5hmC), 5-formylcytosine (5fC), 5-carboxylcytosine (5caC), 5-glucosylhydroxymethylcytosine, 3-methylcytosine (3mC), N4-methylcytosine (4mC) or combinations thereof. Exemplary modifications recognized modification-sensitive restriction enzymes that specifically cleave recognition sites comprising one or more modified nucleotides include modifications mediated by Dam, Dcm, EcoKI, DNMT1, DNMT3A, DNMT3B and TET enzymes.

Exemplary but non-limiting modification-sensitive restriction enzymes that cleave at a recognition site comprising one or more modified nucleotides within or adjacent to the recognition site include, but are not limited to AbaSI, DpnI, FspEI, LpnPI, MspJI and McrBC.

In some embodiments, the modification comprises 5-glucosylhydroxymethylcytosine and the modification-sensitive restriction enzyme comprises AbaSI. AbaSI cleaves an AbaSI recognition site comprising a glucosylhydroxymethylcytosine, and does not cleave an AbaSI recognition site that does not comprise a glucosylhydroxymethylcytosine.

In some embodiments, the nucleotide modification comprises 5-hydroxymethylcytosine and the modification-sensitive restriction enzyme comprises AbaSI and T4 phage β-glucosyltransferase. T4 Phage β-glucosyltransferase specifically transfers the glucose moiety of uridine diphosphoglucose (UDP-Glc) to the 5-hydroxymethylcytosine (5-hmC) residues in double-stranded DNA, for example, within the AbaSI recognition site, making a glucosylhydroxymethylcytosine modified AbaSI recognition site. AbaSI cleaves an AbaSI recognition site comprising glucosylhydroxymethylcytosine and does not cleave an AbaSI recognition site that does not comprise a glucosylhydroxymethylcytosine.

In some embodiments, the nucleotide modification comprises methylcytosine and the modification-sensitive restriction enzyme comprises McrBC. McrBC cleaves McrBC sites comprising methylcytosines, and does not cleave McrBC sites that do not comprise methylcytosines. The McrBC site can be modified with methylcytosines on one or both DNA strands. In some embodiments, McrBC also cleaves McrBC sites comprising hydroxymethylcytosines on one or both DNA strands. In some embodiments, the McrBC half sites are separated by up to 3,000 nucleotides. In some embodiments, the McrBC half sites are separated by 55-103 nucleotides.

In some embodiments, the modification comprises adenine methylation and the methods comprise digestion with DpnI. DpnI cleaves a GATC recognition site when the adenines on both strands of the GATC recognition are methylated. In some embodiments, DpnI GATC recognition sites comprising both adenine methylation and cytosine modification occur in bacterial DNA, but not in mammalian DNA. These recognition sites comprising both methylated adenines and modified cytosines can be selectively cleaved by DpnI in a sample (e.g., of mixed bacterial and mammalian DNA), and then treated with T4 polymerase to replace methylated adenines and modified cytosines at the cleaved ends with unmodified adenines and cytosines. T4 polymerase catalyzes the synthesis of DNA in the 5′ to 3′ direction, in the presence of a template, primer and nucleotides. T4 polymerase will incorporate unmodified nucleotides into the newly synthesized DNA. This produces a sample that now comprises unmodified cytosines in the nucleic acids of interest and modified cytosines in the nucleic acids targeted for depletion. These differences in modified cytosines can be used to enrich for nucleic acids of interest using the methods of the disclosure.

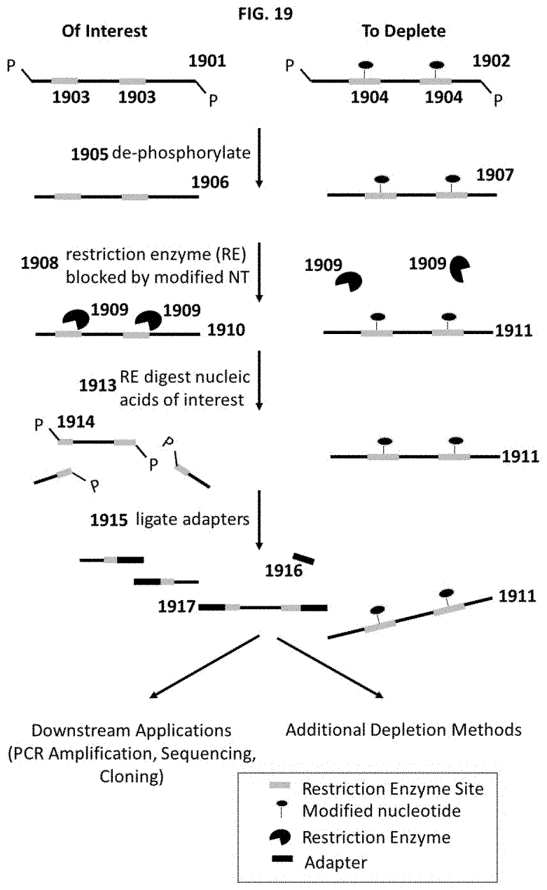

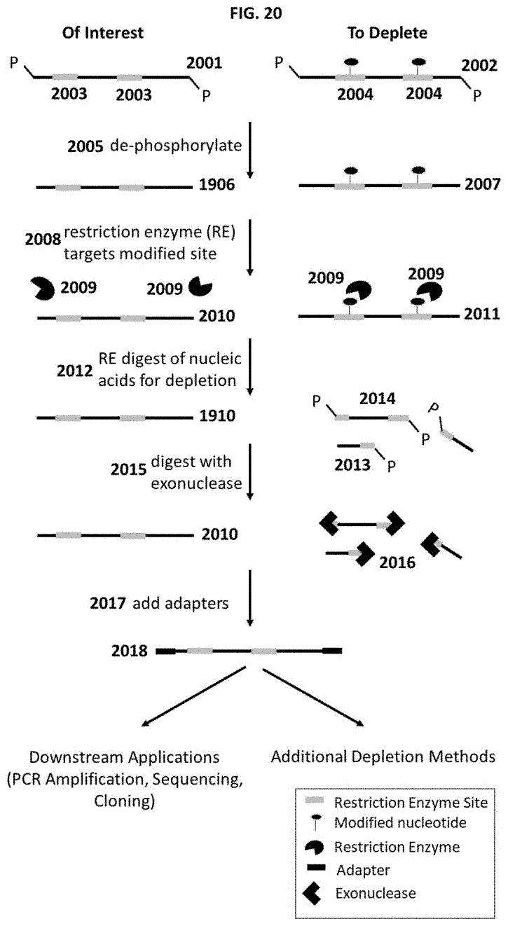

In some embodiments of the methods of the disclosure, the nucleic acids in the sample are terminally dephosphorylated, so that contacting the nucleic acids in the sample with a modification-sensitive restriction enzyme produces either nucleic acids of interest or nucleic acids targeted for depletion with exposed terminal phosphates than can be used in the methods of the disclosure to enrich the sample for nucleic acids of interest. For example, these exposed terminal phosphates can be used to target the nucleic acids for depletion for degradation by an exonuclease (FIG. 20 or the nucleic acids of interest for adapter ligation (FIG. 19).

As used herein, the term “terminally dephosphorylated” refers to nucleic acids that have had the terminal phosphate groups removed from the 5′ and 3′ ends of the nucleic acid molecule. In some embodiments, the nucleic acids in the sample are terminally dephosphorylated using a phosphatase, such as an alkaline phosphatase. Exemplary phosphatases of the disclosure include, but are not limited to shrimp alkaline phosphatase (SAP), recombinant shrimp alkaline phosphatase (rSAP), calf intestine alkaline phosphatase (CIP) and Antarctic phosphatase.

As used herein, the term “exonuclease” refers to a class of enzymes successively remove nucleotides from the 3′ or 5′ ends of a nucleic acid molecule. The nucleic acid molecule can be DNA or RNA. The DNA or RNA can be single stranded or double stranded. Exemplary exonucleases include, but are not limited to Lambda nuclease, Exonuclease I, Exonuclease III and BAL-31. Exonucleases can be used to selectively degrade nucleic acids targeted for depletion using the methods of the disclosure (FIG. 20, e.g.).

The disclosure provides adapters that attached to the 5′ and 3′ ends of the nucleic acids in the sample or the nucleic acids of interest using the methods described herein.

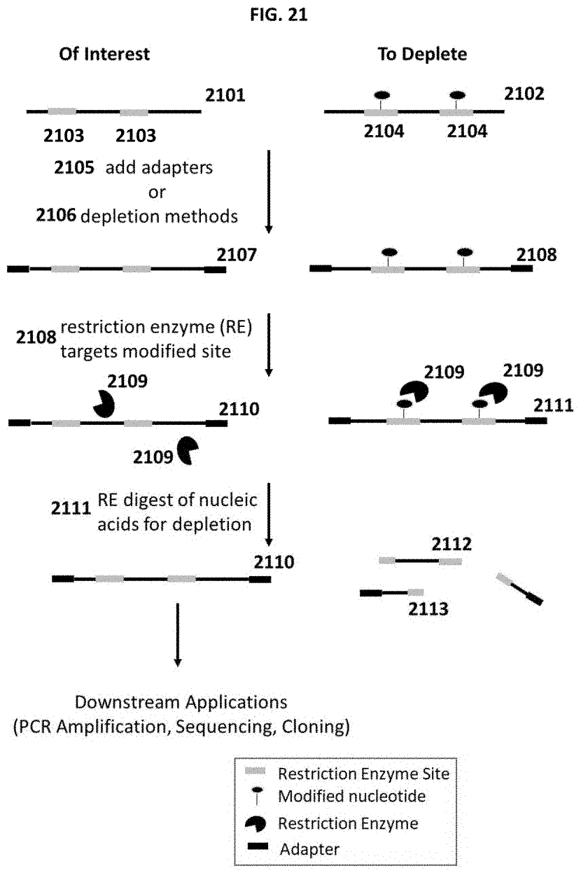

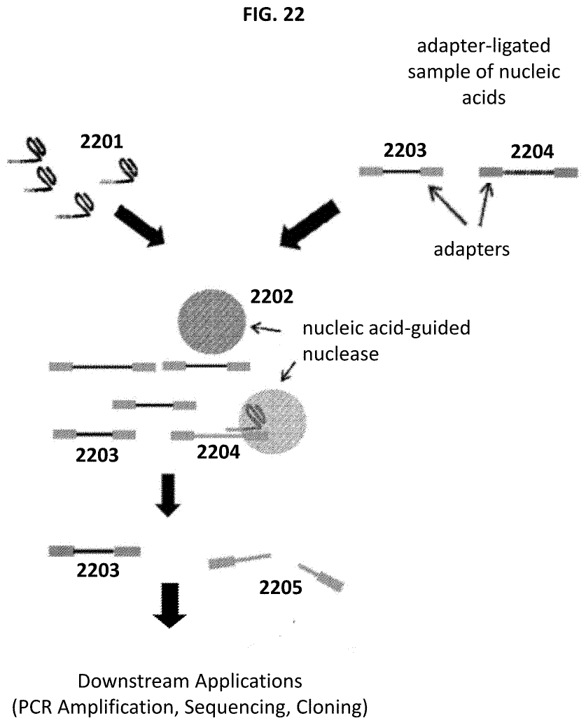

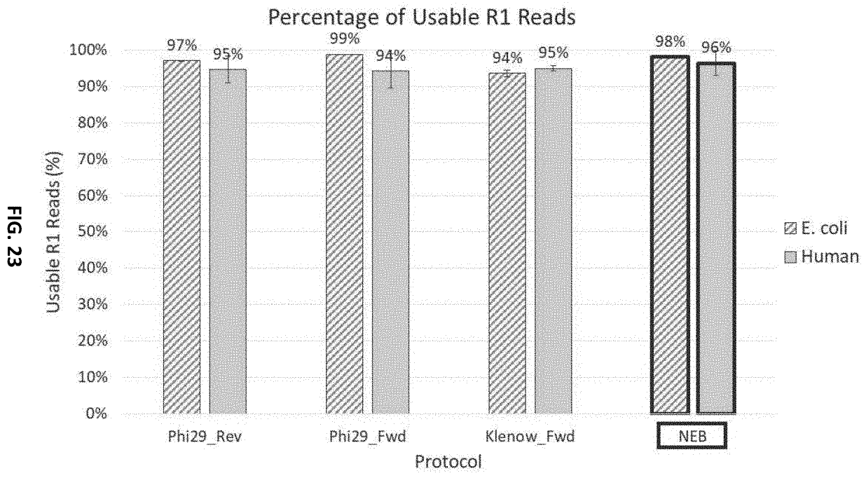

In some embodiments of the methods of the disclosure, adapters are attached to all the nucleic acids in the sample, for example using the methods of library preparation described herein, and then differences in nucleotide modification are used to selectively cleave the nucleic acids targeted for depletion, producing nucleic acids of interest that have adapters on both ends and nucleic acids targeted for depletion that adapters on one end or no adapters (FIG. 21, FIG. 22). In some embodiments, differences in nucleotide modification are used to selectively deplete the nucleic acids targeted for depletion, and then adapters are attached to the target nucleic acids of interest (FIG. 20).

The nucleic acids targeted for depletion can be depleted by differential adapter attachment. In some embodiments, adapters are attached to nucleic acids of a sample, and subsequently one or more adapters are removed from nucleic acids targeted for depletion based on their modification status. For example, nucleic acids targeted for depletion with adapters attached to both ends can be cleaved by a modification-sensitive restriction enzyme, thereby producing nucleic acids targeted for depletion with adapters attached to only one end. Subsequent steps (e.g., amplification) can be used to target only nucleic acids with adapters attached to both ends, thereby depleting the nucleic acids targeted for depletion. In another example, the nucleic acids of the sample are treated (e.g., by dephosphorylation) such that only cleaved nucleic acids are able to have adapters attached; subsequently, nucleic acids of interest can be cleaved by a modification-sensitive restriction enzyme (e.g., thereby exposing a phosphate group) and adapters can be attached. Subsequent steps (e.g., amplification) can be used to target only nucleic acids with adapters attached, thereby depleting the nucleic acids targeted for depletion.

The nucleic acids targeted for depletion can be depleted by digestion, such as digestion with an exonuclease.

The nucleic acids targeted for depletion can be depleted by size selection. For example, a modification-sensitive restriction enzyme can be used to cleave either the nucleic acids of interest or the nucleic acids targeted for depletion, and subsequently the nucleic acids of interest can be separated from the nucleic acids targeted for depletion based on size differences due to the cleavage.

In some cases, the nucleic acids targeted for depletion are depleted without the use of size selection.

Protocol 1: Exemplary methods of the application described herein are depicted in FIG. 19. A sample of nucleic acids comprising target nucleic acids of interest (1901) and nucleic acids targeted for depletion (1902), is terminally dephosphorylated (1905) to produce unphosphorylated nucleic acids of interest (1906) and nucleic acids targeted for depletion (1907). In some embodiments, the nucleic acids are fragmented prior to dephosphorylation. In some embodiments, the nucleic acids in the sample are terminally dephosphorylated with a phosphatase, for example recombinant shrimp alkaline phosphatase (rSAP). In some embodiments, both the nucleic acids of interest and the nucleic acids targeted for depletion comprise one or more recognition sites for a modification-sensitive restriction enzyme (1903, 1904, respectively). In the nucleic acids of interest, the recognition sites for the modification-sensitive restriction enzyme do not comprise modified nucleotides (1903), or alternatively, contain modified nucleotides less frequently than the corresponding recognition sites of the nucleic acids targeted for depletion. In the nucleic acids targeted for depletion, the recognition sites for the modification-sensitive restriction enzyme comprise modified nucleotides within or adjacent to the restriction site (1904), or alternatively, comprise modified nucleotides more frequently than the corresponding recognition sites of the nucleic acids of interest. Activity of the modification-sensitive restriction enzyme (1909) is blocked by the presence of modified nucleotides within or adjacent to its cognate recognition site (1908), thereby targeting the activity of the modification-sensitive restriction enzyme to the nucleic acids of interest (compare 1910 and 1911). In some embodiments, the modification-sensitive restriction enzyme (1909) comprises AatII, AccII, Aor13HI, Aor51HI, BspT104I, BssHII, Cfr10I, ClaI, CpoI, Eco52I, HaeII, HapII, HhaI, MluI, NaeI, NotI, NruI, NsbI, PmaCI, Psp1406I, PvuI, SacII, SalI, SmaI, SnaBI, AluI or Sau3AI. In some embodiments, the modification-sensitive restriction enzyme (1909) comprises AluI or Sau3AI. Digesting the sample with the modification-sensitive restriction enzyme (1913) produces nucleic acids of interest with terminal phosphates at the 5′ and 3′ ends of the terminal phosphates (1914). These terminal phosphates are used to ligate adapters (1915, ligation step; 1916, adapters) to the ends of the nucleic acids of interest, producing nucleic acids of interest that are adapter ligated on both ends (1917). In contrast, the nucleic acids targeted for depletion are not adapter ligated (1911). These adapters can be used for downstream applications, for example adapter-mediated PCR amplification, sequencing (e.g. high throughput sequencing), and quantification of the nucleic acids of interest in the sample and/or cloning. This depletes the nucleic acids targeted for depletion by selectively ligating adapters to the nucleic acids of interest. This depletion can be accomplished without the use of size selection. Alternatively, the adapter ligated nucleic acids of interest are subjected to one or more of the additional enrichment methods described herein. For example, the adapter ligated nucleic acids are subjected to additional modification-dependent enrichment methods of the disclosure (for example, the methods depicted in FIG. 21). Alternatively, or in addition, the adapter ligated nucleic acids are subjected to nucleic acid-guided nuclease based enrichment methods of the disclosure (for example, the methods depicted in FIG. 22).

Protocol 2: Exemplary methods of the application described herein are depicted in FIG. 20. A sample of nucleic acids comprising target nucleic acids of interest (2001) and nucleic acids targeted for depletion (2002), is terminally dephosphorylated (2005) to produce unphosphorylated nucleic acids of interest (2006) and nucleic acids targeted for depletion (2007). In some embodiments, the nucleic acids are fragmented prior to dephosphorylation. In some embodiments, the nucleic acids in the sample are terminally dephosphorylated with a phosphatase, for example recombinant shrimp alkaline phosphatase (rSAP). In some embodiments, both the nucleic acids of interest and the nucleic acids targeted for depletion comprise one or more recognition sites for a modification-sensitive restriction enzyme (2003 and 2004, respectively). In the nucleic acids of interest, the recognition sites for the modification-sensitive restriction enzyme do not comprise modified nucleotides (2003), or alternatively, contain modified nucleotides less frequently than the corresponding recognition sites of the nucleic acids targeted for depletion. In the nucleic acids targeted for depletion, the recognition sites for the modification-sensitive restriction enzyme comprise modified nucleotides within or adjacent to the restriction site (2004), or alternatively, comprise modified nucleotides more frequently than the corresponding recognition sites of the nucleic acids of interest. The modification-sensitive restriction enzyme (2009) cuts its cognate recognition site when there are one or more modified nucleotides within or adjacent to the recognition site (2008), and does not cut its cognate recognition site when the recognition site does not comprise one or more modified nucleotides (2008), thereby targeting the activity of the modification-sensitive restriction enzyme to the nucleic acids targeted for depletion (compare 2010 and 2011). In some embodiments, the modification-sensitive restriction enzyme comprises AbaSI, FspEI, LpnPI, MspJI or McrBC. In some embodiments, the modification-sensitive restriction enzyme is FspEI. In some embodiments, the modification-sensitive restriction enzyme is MspJI. Digestion of the sample with the modification-sensitive restriction enzyme (2012) produces nucleic acids targeted for depletion with terminal phosphates one end (2013) or both the 5′ and 3′ ends of the nucleic acid (2014). In contrast, the nucleic acids of interest, which were not cut by the modification-sensitive restriction enzyme, do not have exposed terminal phosphates at the 5′ and or 3′ ends of the nucleic acids (compare 2010 with 2013-2014). The sample is then digested with an exonuclease (2015, digestion step; 2016 exonuclease) which uses the terminal phosphates in the nucleic acids targeted for depletion to remove successive nucleotides from the ends of the nucleic acid molecules, thus depleting the nucleic acids targeted for depletion from the sample. This depletion can be accomplished without the use of size selection. Following exonuclease digestion, adapters are ligated to the nucleic acids of interest (2017), which, lacking terminal phosphates, have not been digested by the exonuclease. This produces nucleic acids of interest that have adapters on both ends (2018). These adapters can be used for downstream applications, for example adapter-mediated PCR amplification, sequencing (e.g. high throughput sequencing), and quantification of the nucleic acids of interest in the sample and/or cloning. Alternatively, the nucleic acids of interest with 5′ and 3′ adapters are subjected to one or more of the additional enrichment methods described herein. For example, the nucleic acids are subjected to additional modification-dependent enrichment methods of the disclosure (for example, the methods depicted in FIG. 21). Alternatively, or in addition, the nucleic acids are subjected to nucleic acid-guided nuclease based enrichment methods of the disclosure (for example, the methods depicted in FIG. 22).

Protocol 3: Exemplary methods of the application described herein are depicted in FIG. 21. A sample of nucleic acids comprising nucleic acids of interest (2101) and nucleic acids targeted for depletion (2102) has adapters attached using the methods described herein (2105), or is subjected to enrichment methods of the disclosure (2106) (e.g., the methods depicted in FIG. 19 or FIG. 20) that produce nucleic acids of interest with adapters (2107) and nucleic acids targeted for depletion with adapters (2108). In some embodiments, both the nucleic acids of interest and the nucleic acids targeted for depletion comprise one or more recognition sites for a modification-sensitive restriction enzyme (2103 and 2104, respectively). In the nucleic acids of interest, the recognition sites for the modification-sensitive restriction enzyme do not comprise modified nucleotides (2103), or alternatively, contain modified nucleotides less frequently than the corresponding recognition sites of the nucleic acids targeted for depletion. In the nucleic acids targeted for depletion, the recognition sites for the modification-sensitive restriction enzyme comprise modified nucleotides within or adjacent to the restriction site (2104), or alternatively, comprise modified nucleotides more frequently than the corresponding recognition sites of the nucleic acids of interest. The modification-sensitive restriction enzyme (2109) cuts its cognate recognition site when there are one or more modified nucleotides within or adjacent to the recognition site (2108), and does not cut its cognate recognition site when the recognition site does not comprise one or more modified nucleotides (2108), thereby targeting the activity of the modification-sensitive restriction enzyme to the nucleic acids targeted for depletion (compare 2110 and 2111). In some embodiments, the modification-sensitive restriction enzyme comprises AbaSI, FspEI, LpnPI, MspJI or McrBC. In some embodiments, the modification-sensitive restriction enzyme is FspEI. In some embodiments, the modification-sensitive restriction enzyme is MspJI. The sample is digested with the modification-sensitive restriction enzyme (2111), producing nucleic acids targeted for depletion that do not have adapters (2112), or have an adapter on only one end (2113). This depletes the nucleic acids targeted for depletion by selectively removing adapters from the nucleic acids targeted for depletion. This depletion can be accomplished without the use of size selection. In contrast, the nucleic acids of interest, which were not cut by the modification-sensitive restriction enzyme, have adapters on both ends (contrast 2110 with 2112-2113). These adapters can be used for downstream applications, for example adapter-mediated PCR amplification, sequencing (e.g. high throughput sequencing), and quantification of the nucleic acids of interest in the sample and/or cloning.

Protocol 4: Exemplary methods of the application described herein are depicted in FIG. 22. A plurality of gNAs (2201) are used to target a nucleic acid-guided nuclease (2202) to nucleic acids targeted for depletion (2203) in a sample of nucleic acids with adapters on both ends. The nucleic acids are generated by any of the methods of enrichment described herein that use modification-sensitive restriction enzymes to deplete nucleic acids targeted for depletion from a sample, either before or after attaching the adapters. In this method, the gNAs are specifically targeted to the nucleic acids targeted for depletion (2203), and not the nucleic acids of interest (2204), which are therefore not cut by the nucleic acid-guided nuclease (2202). Cleavage by the nucleic acid-guided nuclease results in nucleic acids targeted for depletion that have adapters on one end or no adapters (2205), and nucleic acids of interest that have adapters on both ends (2203). These adapters can be used for downstream applications, for example adapter-mediated PCR amplification, sequencing (e.g. high throughput sequencing), quantification of the nucleic acids of interest in the sample and cloning.

Any of the methods described herein can be used as a stand-alone method to deplete nucleic acids targeted for depletion from a sample, thereby enriching for nucleic acids of interest. Alternatively, the methods described herein can be combined to achieve a greater degree of enrichment than any individual method in alone.

While particular combinations of methods, and orders of combinations of methods, are described herein, these are in no way intended to limit the ways in which the methods of the disclosure can be combined. Any method of enriching a sample for nucleic acids of interest of the disclosure that produces nucleic acids of interest with attached adapters as a product of the method can be combined with any additional methods of the disclosure that use nucleic acids with attached adapters as its starting substrate.

### Nucleic Acid-Guided Nuclease Based Enrichment Methods

The disclosure provides nucleic acid-guided nuclease based methods for enriching for sequences of interest in a sample of nucleic acids or a sequencing library produced using the ligation-free methods described herein. The methods of enriching for sequences of interest can optionally be combined other methods of enrichment or depletion described herein. Nucleic acid-guided nuclease based enrichment methods are methods that employ nucleic acid-guided nucleases to enrich a sample for sequences of interest. Nucleic acid-guided nuclease based enrichment methods are described in WO/2016/100955, WO/2017/031360, WO/2017/100343, WO/2017/147345 and WO/2018/227025 the contents of each of which are herein incorporated by reference in their entirety. The contents of International Application No. PCT/US2019/054843, filed on Oct. 4, 2019, and International Application No. PCT/US2019/036102, filed on Jun. 7, 2019, are herein incorporated by reference in their entirety.

The term “nucleic acid-guided nuclease-gNA complex” refers to a complex comprising a nucleic acid-guided nuclease protein and a guide nucleic acid (gNA, for example a gRNA or a gDNA). For example, the “Cas9-gRNA complex” refers to a complex comprising a Cas9 protein and a guide RNA (gRNA). The nucleic acid-guided nuclease may be any type of nucleic acid-guided nuclease, including but not limited to a wild type nucleic acid-guided nuclease, a catalytically dead nucleic acid-guided nuclease, or a nucleic acid-guided nuclease-nickase. When the nucleic-acid guided nuclease is a CRISPR/Cas nucleic-acid guided nuclease, the complex can be referred to as a “CRISPR/Cas system protein-gNA complex.”

Methods of the present disclosure can utilize nucleic acid-guided nucleases. As used herein, a “nucleic acid-guided nuclease” is any nuclease that cleaves DNA, RNA or DNA/RNA hybrids, and which uses one or more guide nucleic acids (gNAs) to confer specificity. Nucleic acid-guided nucleases include CRISPR/Cas system proteins as well as non-CRISPR/Cas system proteins. The nucleic acid-guided nucleases provided herein can be DNA guided DNA nucleases; DNA guided RNA nucleases; RNA guided DNA nucleases; or RNA guided RNA nucleases. The nucleases can be endonucleases. The nucleases can be exonucleases. In one embodiment, the nucleic acid-guided nuclease is a nucleic acid-guided-DNA endonuclease. In one embodiment, the nucleic acid-guided nuclease is a nucleic acid-guided-RNA endonuclease.

In some embodiments, the modification-based enrichment methods and the nucleic acid-guided nuclease based enrichment methods of the disclosure deplete different nucleic acids in the sample, thereby achieving a greater degree of enrichment for the nucleic acids of interest than either approach alone.

Provided herein are pluralities (interchangeably referred to as libraries, or collections) of guide nucleic acids (gNAs).

gNAs can be RNAs (gRNAs) or DNAs (gDNAs) or a mixture of RNA and DNA.

The term “guide nucleic acid” refers to a guide nucleic acid (gNA) that is capable of forming a complex with a nucleic acid guided nuclease, and optionally, additional nucleic acid(s). The gNA may exist as an isolated nucleic acid, or as part of a nucleic acid-guided nuclease-gNA complex, for example a Cas9-gRNA complex.

As used herein, a plurality of gNAs denotes a mixture of gNAs containing at least 10² unique gNAs. In some embodiments a plurality of gNAs contains at least 10² unique gNAs, at least 10³ unique gNAs, at least 10⁴ unique gNAs, at least 10⁵ unique gNAs, at least 10⁶ unique gNAs, at least 10⁷ unique gNAs, at least 10⁸ unique gNAs, at least 10⁹ unique gNAs or at least 10¹⁰ unique gNAs. In some embodiments a collection of gNAs contains a total of at least 10² unique gNAs, at least 10³ unique gNAs, at least 10⁴ unique gNAs or at least 10⁵ unique gNAs.

In some embodiments, a collection of gNAs comprises a first NA segment comprising a targeting sequence; and a second NA segment comprising a nucleic acid-guided nuclease system (e.g., CRISPR/Cas system) protein-binding sequence. In some embodiments, the first and second segments are in 5′- to 3′-order′. In some embodiments, the first and second segments are in 3′- to 5′-order′.

In some embodiments, the size of the first segment varies from 12-100 bp, 12-75 bp, 12-50 bp, 12-30 bp, 12-25 bp, 12-22 bp, 12-20 bp, 12-18 bp, 12-16 bp, 14-250 bp, 14-100 bp, 14-75 bp, 14-50 bp, 14-30 bp, 14-25 bp, 14-22 bp, 14-20 bp, 14-18 bp, 14-17 bp, 14-16 bp, 15-250 bp, 15-100 bp, 15-75 bp, 15-50 bp, 15-30 bp, 15-25 bp, 15-22 bp, 15-20 bp, 15-18 bp, 15-17 bp, 15-16 bp, 16-250 bp, 16-100 bp, 16-75 bp, 16-50 bp, 16-30 bp, 16-25 bp, 16-22 bp, 16-20 bp, 16-18 bp, 16-17 bp, 17-250 bp, 17-100 bp, 17-75 bp, or 17-50 bp, 17-30 bp, 17-25 bp, 17-22 bp, 17-20 bp, 17-18 bp, 18-250 bp, 18-100 bp, 18-75 bp, 18-50 bp, 18-30 bp, 18-25 bp, 18-22 bp, 18-20 bp, 19-250 bp, 19-100 bp, 19-75 bp, or 19-50 bp, 19-30 bp, 19-25 bp, or 19-22 bp across the plurality of gNAs. In some particular embodiments, the size of the first segment is 15 bp, 16 bp, 17 bp, 18 bp, 19 bp, or 20 bp.

In some embodiments, at least 10%, or at least 15%, or at last 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or at least 95%, or 100% of the first segments in the plurality are 15-50 bp.

In some embodiments, the plurality of gNAs comprises targeting sequences which can base-pair with a target sequence in the nucleic acids targeted for depletion, wherein the target sequence in the nucleic acids targeted for depletion is spaced at least every 1 bp, at least every 2 bp, at least every 3 bp, at least every 4 bp, at least every 5 bp, at least every 6 bp, at least every 7 bp, at least every 8 bp, at least every 9 bp, at least every 10 bp, at least every 11 bp, at least every 12 bp, at least every 13 bp, at least every 14 bp, at least every 15 bp, at least every 16 bp, at least every 17 bp, at least every 18 bp, at least every 19 bp, 20 bp, at least every 25 bp, at least every 30 bp, at least every 40 bp, at least every 50 bp, at least every 100 bp, at least every 200 bp, at least every 300 bp, at least every 400 bp, at least every 500 bp, at least every 600 bp, at least every 700 bp, at least every 800 bp, at least every 900 bp, at least every 1000 bp, at least every 2500 bp, at least every 5000 bp, at least every 10,000 bp, at least every 15,000 bp, at least every 20,000 bp, at least every 25,000 bp, at least every 50,000 bp, at least every 100,000 bp, at least every 250,000 bp, at least every 500,000 bp, at least every 750,000 bp, or even at least every 1,000,000 bp across a genome or transcriptome targeted for depletion in the sample.

In some embodiments, the plurality of gNAs comprises a first NA segment comprising a targeting sequence; and a second NA segment comprising a nucleic acid-guided nuclease system (e.g., CRISPR/Cas system) protein-binding sequence, wherein the gNAs in the plurality can have a variety of second NA segments with various specificities for protein members of the nucleic acid-guided nuclease system (e.g., CRISPR/Cas system). For example a collection of gNAs as provided herein, can comprise members whose second segment comprises a nucleic acid-guided nuclease system (e.g., CRISPR/Cas system) protein-binding sequence specific for a first nucleic acid-guided nuclease system (e.g., CRISPR/Cas system) protein; and also comprises members whose second segment comprises a nucleic acid-guided nuclease system (e.g., CRISPR/Cas system) protein-binding sequence specific for a second nucleic acid-guided nuclease system (e.g., CRISPR/Cas system) protein, wherein the first and second nucleic acid-guided nuclease system (e.g., CRISPR/Cas system) proteins are not the same. In some embodiments, a plurality of gNAs as provided herein comprises members that exhibit specificity for a Cas9 protein and another protein selected from the group consisting of Cpf1, Cas3, Cas8a-c, Cas10, CasX, CasY, Cas13, Cas14, Cse1, Csy1, Csn2, Cas4, Csm2, and Cm5. The order of the first NA segment comprising a targeting sequence and the second NA segment comprising a nucleic acid-guided nuclease system protein-binding sequence will depend on the nucleic acid-guided nuclease system protein. The appropriate 5′ to 3′ arrangement of the first and second NA segments and choice of nucleic acid-guided nuclease system proteins will be apparent to one of ordinary skill in the art.

In some embodiments the gNAs comprise DNA and RNA. In some embodiments, the gNAs consist of DNA (gDNAs). In some embodiments, the gNAs consist of RNA (gRNAs).

In some embodiments, the gNA comprises a gRNA and the gRNA comprises two sub-segments, which encode for a crRNA and a tracrRNA. In some embodiment, the crRNA does not comprise the targeting sequences plus the extra sequence which can hybridize with tracrRNA. In some embodiments, the crRNA comprises an extra sequence which can hybridize with tracrRNA. In some embodiments, the two sub-segments are independently transcribed. In some embodiments, the two sub-segments are transcribed as a single unit. In some embodiments, the DNA encoding the crRNA comprises the targeting sequence 5′ of the sequence GTTTTAGAGCTATGCTGTTTTG (SEQ ID NO:6). In some embodiments, the DNA encoding the tracrRNA comprises the sequence GGAACCATTCAAAACAGCATAGCAAGTTAAAATAAGGCTAGTCCGTTATCAACT TGAAAAAGTGGCACCGAGTCGGTGCTTTTTTT (SEQ ID NO:7).

As used herein, a targeting sequence is one that directs the gNA to a target sequence in a nucleic acid targeted for depletion in a sample. For example, a targeting sequence targets any of the non-host sequences described herein.

Provided herein are gNAs and pluralities of gNAs that comprise a segment that comprises a targeting sequence.

In some embodiments, the targeting sequence comprises or consists of DNA. In some embodiments, the targeting sequence comprises or consists of RNA.

In some embodiments, the targeting sequence comprises RNA, and shares at least 70% sequence identity, at least 75% sequence identity, at least 80% sequence identity, at least 85% sequence identity, at least 90% sequence identity, at least 95% sequence identity, or shares 100% sequence identity to a sequence 5′ to a PAM sequence on a sequence of interest, except that the RNA comprises uracils instead of thymines. In some embodiments, the targeting sequence comprises RNA, and shares at least 70% sequence identity, at least 75% sequence identity, at least 80% sequence identity, at least 85% sequence identity, at least 90% sequence identity, at least 95% sequence identity, or shares 100% sequence identity to a sequence 3′ to a PAM sequence on a sequence of interest, except that the RNA comprises uracils instead of thymines. In some embodiments, the PAM sequence is AGG, CGG, TGG, GGG or NAG. In some embodiments, the PAM sequence is TTN, TCN or TGN.

In some embodiments, the targeting sequence comprises RNA and is complementary to the strand opposite to a sequence of nucleotides 5′ to a PAM sequence. In some embodiments, the targeting sequence is at least 70% complementary, at least 75% complementary, at least 80% complementary, at least 85% complementary, at least 90% complementary, at least 95% complementary, or is 100% complementary to the strand opposite to a sequence of nucleotides 5′ to a PAM sequence. In some embodiments, the targeting sequence comprises RNA and is complementary to the strand opposite to a sequence of nucleotides 3′ to a PAM sequence. In some embodiments, the targeting sequence is at least 70% complementary, at least 75% complementary, at least 80% complementary, at least 85% complementary, at least 90% complementary, at least 95% complementary, or is 100% complementary to the strand opposite to a sequence of nucleotides 3′ to a PAM sequence. In some embodiments, the PAM sequence is AGG, CGG, TGG, GGG or NAG. In some embodiments, the PAM sequence is TTN, TCN or TGN.

Different CRISPR/Cas system proteins recognize different PAM sequences. PAM sequences can be located 5′ or 3′ of a targeting sequence. For example, Cas9 can recognize an NGG PAM located on the immediate 3′ end of a targeting sequence. Cpf1 can recognize a TTN PAM located on the immediate 5′ end of a targeting sequence. All PAM sequences recognized by all CRISPR/Cas system proteins are envisaged as being within the scope of the disclosure. It will be readily apparent to one of ordinary skill in the art which PAM sequences are compatible with a particular CRISPR/Cas system protein.

Provided herein are gNAs and pluralities of gNAs comprising a segment that comprises a nucleic acid-guided nuclease protein-binding sequence. The nucleic acid-guided nuclease can be a nucleic acid-guided nuclease system protein (e.g., CRISPR/Cas system). A nucleic acid-guided nuclease system can be an RNA-guided nuclease system. A nucleic acid-guided nuclease system can be a DNA-guided nuclease system.

A nucleic acid-guided nuclease protein-binding sequence is a nucleic acid sequence that binds any protein member of a nucleic acid-guided nuclease system. For example, a CRISPR/Cas protein-binding sequence is a nucleic acid sequence that binds any protein member of a CRISPR/Cas system.

In some embodiments, CRISPR/Cas system proteins can be from any bacterial or archaeal species. In some embodiments, the CRISPR/Cas system protein is isolated, recombinantly produced, or synthetic. In some embodiments, examples of CRISPR/Cas system proteins can be naturally occurring or engineered versions.

In some embodiments, nucleic acid-guided nuclease system proteins (e.g., CRISPR/Cas system proteins) can be from any bacterial or archaeal species. In some embodiments, naturally occurring CRISPR/Cas system proteins can belong to CAS Class I Type I, III, or IV, or CAS Class II Type II or V, and can include Cas9, Cas3, Cas8a-c, Cas10, CasX, CasY, Cas13, Cas14, Cse1, Csy1, Csn2, Cas4, Csm2, Cmr5, Csf1, C2c2, and Cpf1. In an exemplary embodiment, the CRISPR/Cas system protein comprises Cas9. In an exemplary embodiment, the CRISPR/Cas system protein comprises Cpf1.

In some embodiments, the nucleic acid-guided nuclease system proteins (e.g., CRISPR/Cas system proteins) are from, or are derived from nucleic acid-guided nuclease system proteins (e.g., CRISPR/Cas system proteins) from Streptococcus pyogenes, Staphylococcus aureus, Neisseria meningitidis, Streptococcus thermophiles, Treponema denticola, Francisella tularensis, Pasteurella multocida, Campylobacter jejuni, Campylobacter lari, Mycoplasma gallisepticum, Nitratifractor salsuginis, Parvibaculum lavamentivorans, Roseburia intestinalis, Neisseria cinerea, Gluconacetobacter diazotrophicus, Azospirillum, Sphaerochaeta globus, Flavobacterium columnare, Fluviicola taffensis, Bacteroides coprophilus, Mycoplasma mobile, Lactobacillus farciminis, Streptococcus pasteurianus, Lactobacillus johnsonii, Staphylococcus pseudintermedius, Filfactor alocis, Legionella pneumophila, Suterella wadsworthensis Corynebacter diphtheria, Acidaminococcus, Lachnospiraceae bacterium or Prevotella.

In some embodiments, the nucleic acid-guided nuclease system protein-binding sequence comprises a gNA (e.g., gRNA) stem-loop sequence. Different CRISPR/Cas system proteins are compatible with different nucleic acid-guided nuclease system protein-binding sequences. It will be readily apparent to one of ordinary skill in the art which CRISPR/Cas system proteins are compatible with which nucleic acid-guided nuclease system protein-binding sequences.

In some embodiments, a double-stranded DNA sequence encoding the gNA (e.g., gRNA) stem-loop sequence comprises the following DNA sequence on one strand (5′>3′, GTTTTAGAGCTAGAAATAGCAAGTTAAAATAAGGCTAGTCCGTTATCAACTTGAA AAAGTGGCACCGAGTCGGTGCTTTTTTT) (SEQ ID NO:8), and its reverse-complementary DNA on the other strand (5′>3′, AAAAAAAGCACCGACTCGGTGCCACTTTTTCAAGTTGATAACGGACTAGCCTTAT TTTAACTTGCTATTTCTAGCTCTAAAAC) (SEQ ID NO:9).

In some embodiments, a single-stranded DNA sequence encoding the gNA (e.g., gRNA) stem-loop sequence comprises the following DNA sequence: (5′>3′, AAAAAAAGCACCGACTCGGTGCCACTTTTTCAAGTTGATAACGGACTAGCCTTAT TTTAACTTGCTATTTCTAGCTCTAAAAC) (SEQ ID NO:10), wherein the single-stranded DNA serves as a transcription template.

In some embodiments, the gNA (e.g., gRNA) stem-loop sequence comprises the following RNA sequence: (5′>3′, GUUUUAGAGCUAGAAAUAGCAAGUUAAAAUAAGGCUAGUCCGUUAUCAACUU GAAAAAGUGGCACCGAGUCGGUGCUUUUUUU) (SEQ ID NO:11).

In some embodiments, a double-stranded DNA sequence encoding the gNA (e.g., gRNA) stem-loop sequence comprises the following DNA sequence on one strand (5′>3′, GTTTTAGAGCTATGCTGGAAACAGCATAGCAAGTTAAAATAAGGCTAGTCCGTTA TCAACTTGAAAAAGTGGCACCGAGTCGGTGCTTTTTTTC) (SEQ ID NO:12), and its reverse-complementary DNA on the other strand (5′>3′, GAAAAAAAGCACCGACTCGGTGCCACTTTTTCAAGTTGATAACGGACTAGCCTTA TTTTAACTTGCTATGCTGTTTCCAGCATAGCTCTAAAAC) (SEQ ID NO:13).

In some embodiments, a single-stranded DNA sequence encoding the gNA (e.g., gRNA) stem-loop sequence comprises the following DNA sequence: (5′>3′, GAAAAAAAGCACCGACTCGGTGCCACTTTTTCAAGTTGATAACGGACTAGCCTTA TTTTAACTTGCTATGCTGTTTCCAGCATAGCTCTAAAAC) (SEQ ID NO:14), wherein the single-stranded DNA serves as a transcription template.

In some embodiments, the gNA (e.g., gRNA) stem-loop sequence comprises the following RNA sequence: (5′>3′, GUUUUAGAGCUAUGCUGGAAACAGCAUAGCAAGUUAAAAUAAGGCUAGUCCG UUAUCAACUUGAAAAAGUGGCACCGAGUCGGUGCUUUUUUUC) (SEQ ID NO:15).

In some embodiments, the CRISPR/Cas system protein is a Cpf1 protein. In some embodiments, the Cpf1 protein is isolated or derived from Franciscella species or Acidaminococcus species. In some embodiments, the gNA (e.g., gRNA) CRISPR/Cas system protein-binding sequence comprises the following RNA sequence: (5′>3′, AAUUUCUACUGUUGUAGAU) (SEQ ID NO:16).

In some embodiments, the CRISPR/Cas system protein is a Cpf1 protein. In some embodiments, the Cpf1 protein is isolated or derived from Franciscella species or Acidaminococcus species. In some embodiments, a DNA sequence encoding the gNA (e.g., gRNA) CRISPR/Cas system protein-binding sequence comprises the following DNA sequence: (5′>3′, AATTTCTACTGTTGTAGAT) (SEQ ID NO:17). In some embodiments, the DNA is single stranded. In some embodiments, the DNA is double stranded.

In some embodiments, provided herein is a gNA (e.g., gRNA) comprising a first NA segment comprising a targeting sequence and a second NA segment comprising a nucleic acid-guided nuclease (e.g., CRISPR/Cas) system protein-binding sequence. In some embodiments, the size of the first segment is 15 bp, 16 bp, 17 bp, 18 bp, 19 bp or 20 bp. In some embodiments, the second segment comprises a single segment, which comprises the gRNA stem-loop sequence. In some embodiments, the gRNA stem-loop sequence comprises the following RNA sequence: (5′>3′, GUUUUAGAGCUAGAAAUAGCAAGUUAAAAUAAGGCUAGUCCGUUAUCAACUU GAAAAAGUGGCACCGAGUCGGUGCUUUUUUU) (SEQ ID NO:18). In some embodiments, the gRNA stem-loop sequence comprises the following RNA sequence: (5′>3′, GUUUUAGAGCUAUGCUGGAAACAGCAUAGCAAGUUAAAAUAAGGCUAGUCCG UUAUCAACUUGAAAAAGUGGCACCGAGUCGGUGCUUUUUUUC) (SEQ ID NO:19). In some embodiments, the second segment comprises two sub-segments: a first RNA sub-segment (crRNA) that forms a hybrid with a second RNA sub-segment (tracrRNA), which together act to direct nucleic acid-guided nuclease (e.g., CRISPR/Cas) system protein binding. In some embodiments, the sequence of the second sub-segment comprises GUUUUAGAGCUAUGCUGUUUUG (SEQ ID NO:20). In some embodiments, the first RNA segment and the second RNA segment together form a crRNA sequence. In some embodiments, the other RNA that will form a hybrid with the second RNA segment is a tracrRNA. In some embodiments the tracrRNA comprises the sequence of 5′>3′, GGAACCAUUCAAAACAGCAUAGCAAGUUAAAAUAAGGCUAGUCCGUUAUCAA CUUGAAAAAGUGGCACCGAGUCGGUGCUUUUUUU (SEQ ID NO:21)

In some embodiments, provided herein is a gNA (e.g., gRNA) comprising a first NA segment comprising a targeting sequence and a second NA segment comprising a nucleic acid-guided nuclease (e.g., CRISPR/Cas) system protein-binding sequence. In some embodiments, for example those embodiments wherein the CRISPR/Cas system protein is a Cpf1 system protein, the second segment is 5′ of the first segment. In some embodiments, the size of the first segment is 20 bp. In some embodiments, the size of the first segment is greater than 20 bp. In some embodiments, the size of the first segment is greater than 30 bp. In some embodiments, the second segment comprises a single segment, which comprises the gRNA stem-loop sequence. In some embodiments, the gRNA stem-loop sequence comprises the following RNA sequence: (5′>3′, AAUUUCUACUGUUGUAGAU) (SEQ ID NO:22).

A CRISPR/Cas system protein may be at least 60% identical (e.g., at least 70%, at least 80%, or 90% identical, at least 95% identical or at least 98% identical or at least 99% identical) to a wild type CRISPR/Cas system protein. The CRISPR/Cas system protein may have all the functions of a wild type CRISPR/Cas system protein, or only one or some of the functions, including binding activity and nuclease activity.

The term “CRISPR/Cas system protein-associated guide NA” refers to a guide NA (gNA). The CRISPR/Cas system protein-associated guide NA may exist as isolated NA, or as part of a CRISPR/Cas system protein-gNA complex.

In some embodiments, the CRISPR/Cas system protein is an RNA-guided DNA nuclease. In some embodiments, the DNA cleaved by the CRISPR/Cas system protein is double stranded. Exemplary RNA-guided DNA nucleases that cut double stranded DNA include, but are not limited to Cas9, Cpf1, CasX and CasY. Further exemplary RNA-guided DNA nucleases include Cas10, Csm2, Csm3, Csm4, and Csm5. In some embodiments, Cas10, Csm2, Csm3, Csm4, and Csm5 form a ribonucleoprotein complex with a gRNA. In some embodiments, the CRISPR/Cas System protein nucleic acid-guided nuclease is or comprises Cas9. The Cas9 of the present disclosure can be isolated, recombinantly produced, or synthetic. In some embodiments, the Cas9 protein is thermostable. Examples of Cas9 proteins that can be used in the embodiments herein can be found in F. A. Ran, L. Cong, W. X. Yan, D. A. Scott, J. S. Gootenberg, A. J. Kriz, B. Zetsche, O. Shalem, X. Wu, K. S. Makarova, E. V. Koonin, P. A. Sharp, and F. Zhang; “In vivo genome editing using Staphylococcus aureus Cas9,” Nature 520, 186-191 (9 Apr. 2015) doi:10.1038/nature14299, which is incorporated herein by reference. In some embodiments, the Cas9 is a Type II CRISPR system derived from Streptococcus pyogenes, Staphylococcus aureus, Neisseria meningitidis, Streptococcus thermophiles, Treponema denticola, Francisella tularensis, Pasteurella multocida, Campylobacter jejuni, Campylobacter lari, Mycoplasma gallisepticum, Nitratifractor salsuginis, Parvibaculum lavamentivorans, Roseburia intestinalis, Neisseria cinerea, Gluconacetobacter diazotrophicus, Azospirillum, Sphaerochaeta globus, Flavobacterium columnare, Fluviicola taffensis, Bacteroides coprophilus, Mycoplasma mobile, Lactobacillus farciminis, Streptococcus pasteurianus, Lactobacillus johnsonii, Staphylococcus pseudintermedius, Filfactor alocis, Legionella pneumophila, Suterella wadsworthensis, or Corynebacter diphtheria.

In some embodiments, the Cas9 is a Type II CRISPR system derived from S. pyogenes and the PAM sequence is NGG located on the immediate 3′ end of the target specific guide sequence. The PAM sequences of Type II CRISPR systems from exemplary bacterial species can also include: Streptococcus pyogenes (NGG), Staph aureus (NNGRRT), Neisseria meningitidis (NNNNGATT), Streptococcus thermophilus (NNAGAA) and Treponema denticola (NAAAAC), which are all usable without deviating from the present disclosure.

In one exemplary embodiment, Cas9 sequence can be obtained, for example, from the pX330 plasmid (available from Addgene), re-amplified by PCR then cloned into pET30 (from EMID biosciences) to express in bacteria and purify the recombinant 6His tagged protein.

A “Cas9-gNA complex” refers to a complex comprising a Cas9 protein and a guide NA. A Cas9 protein may be at least 60% identical (e.g., at least 70%, at least 80%, or 90% identical, at least 95% identical or at least 98% identical or at least 99% identical) to a wild type Cas9 protein, e.g., to the Streptococcus pyogenes Cas9 protein. The Cas9 protein may have all the functions of a wild type Cas9 protein, or only one or some of the functions, including binding activity, nuclease activity, and nuclease activity.

The term “Cas9-associated guide NA” refers to a guide NA as described above. The Cas9-associated guide NA may exist isolated, or as part of a Cas9-gNA complex.

In some embodiments, the CRISPR/Cas system protein nucleic acid-guided nuclease is or comprises a Cpf1 system protein. Cpf1 system proteins of the present disclosure can be isolated, recombinantly produced, or synthetic. In some embodiments, the Cpf1 protein is thermostable.

Cpf1 system proteins are Class II, Type V CRISPR system proteins. In some embodiments, the Cpf1 protein is isolated or derived from Francisella tularensis. In some embodiments, the Cpf1 protein is isolated or derived from Acidaminococcus, Lachnospiraceae bacterium or Prevotella.

Cpf1 system proteins bind to a single guide RNA comprising a nucleic acid-guided nuclease system protein-binding sequence (e.g., stem-loop) and a targeting sequence. The Cpf1 targeting sequence comprises a sequence located immediately 3′ of a Cpf1 PAM sequence in a target nucleic acid. Unlike Cas9, the Cpf1 nucleic acid-guided nuclease system protein-binding sequence is located 5′ of the targeting sequence in the Cpf1 gRNA. Cpf1 can also produce staggered rather than blunt ended cuts in a target nucleic acid. Following targeting of the Cpf1 protein-gRNA protein complex to a target nucleic acid, Francisella derived Cpf1, for example, cleaves the target nucleic acid in a staggered fashion, creating an approximately 5 nucleotide 5′ overhang 18-23 bases away from the PAM at the 3′ end of the targeting sequence. In contrast, cutting by a wild type Cas9 produces a blunt end 3 nucleotides upstream of the Cas9 PAM.

An exemplary Cpf1 gRNA stem-loop sequence comprises the following RNA sequence: (5′>3′, AAUUUCUACUGUUGUAGAU) (SEQ ID NO:23).

A “Cpf1 protein-gNA complex” refers to a complex comprising a Cpf1 protein and a guide NA (e.g. a gRNA). Where the gNA is a gRNA, the gRNA may be composed of a single molecule, i.e., one RNA (“crRNA”) which hybridizes to a target and provides sequence specificity.

A Cpf1 protein may be at least 60% identical (e.g., at least 70%, at least 80%, or 90% identical, at least 95% identical or at least 98% identical or at least 99% identical) to a wild type Cpf1 protein. The Cpf1 protein may have all the functions of a wild type Cpf1 protein, or only one or some of the functions, including binding activity and nuclease activity.

Cpf1 system proteins recognize a variety of PAM sequences. Exemplary PAM sequences recognized by Cpf1 system proteins include, but are not limited to TTN, TCN and TGN. Additional Cpf1 PAM sequences include, but are not limited to TTTN. One feature of Cpf1 PAM sequences is that they have a higher A/T content than the NGG or NAG PAM sequences used by Cas9 proteins.

### Kits and Articles of Manufacture

The present application provides kits comprising any one or more of the compositions described herein, not limited to adapters, enzymes gRNAs, gRNA collections, nucleic acid molecules encoding the gRNA collections, and the like.

In exemplary embodiments, the kit comprises a first adapter, a second adapter, indexing primers, enzymes, control samples and instructions for use in preparing libraries from nucleic acid samples using the methods described herein. In some embodiments, the nucleic acids samples are degraded or comprise small nucleic acid fragments (e.g., less than 50 bp in length).

In exemplary embodiments, the kit comprises a collection of DNA molecules capable of transcribing into a library of gRNAs wherein the gRNAs are targeted to human genomic or other sources of DNA sequences.

In one embodiment, the kit comprises a collection of gRNAs wherein the gRNAs are targeted to human genomic or other sources of DNA sequences.

In some embodiments, provided herein are kits comprising any of the collection of nucleic acids encoding gRNAs, as described herein. In some embodiments, provided herein are kits comprising any of the collection of gRNAs, as described herein.

The present application also provides all essential reagents and instructions for carrying out the methods of making the gRNAs and the collection of nucleic acids encoding gRNAs, as described herein. In some embodiments, provided herein are kits that comprise all essential reagents and instructions for carrying out the methods of making individual gRNAs and collections of gRNAs as described herein.

Also provided herein is computer software monitoring the information before and after contacting a sample with a gRNA collection produced herein. In one exemplary embodiment, the software can compute and report the abundance of non-target sequence in the sample before and after providing gRNA collection to ensure no off-target targeting occurs, and wherein the software can check the efficacy of targeted-depletion/encrichment/capture/partitioning/labeling/regulation/editing by comparing the abundance of the target sequence before and after providing gRNA collection to the sample.

### EXAMPLES

### Example 1: Protocol for Ligation-Free Library Preparation

A short PCR product was used to produce a sequenceable library using the following protocol:

Protocol Overview

Part 1—Blunt Ending

The PCR product was blunt ended using T4 DNA polymerase. The ends of the DNA need to be blunt for T4 DNA polymerases such as Klenow to efficiently add dNTPs or ddNTPs.

Following blunt ending, QiaQuick cleanup was used to remove remaining nucleotides. Optionally, recombinant shrimp alkaline phosphatase (rSAP) enzymatic cleanup, a bead based cleanup or other column can be used to remove nucleotides at this point.

Part 2—Blocking

3′ end blocking was carried out using ddNTPs and Klenow. Sequencing suggests that this step, and therefore perhaps also the blunt ending step, may not be necessary. Most sequences after sequencing were unblocked, indicating that the blocking step may not be necessary. If the blunt ending is needed, but not the blocking, since the enzyme is heat denatured, it may be possible to skip the post-blunting purification prior to this step.

Following 3′ end blocking, QiaQuick cleanup was used to remove remaining nucleotides. Optionally, rSAP enzymatic cleanup, a bead based cleanup or other column can be used to remove nucleotides at this point.

Note: The initial sequencing results indicates that this step (and therefore even the blunt end step) may not be necessary.

Part 3—Adapter 1 Addition

A single-sided PCR (i.e., with only one primer) that allows the adapter+primer to anneal and extend the length of the DNA was carried out. Initially, this step was carried out with Taq polymerase. However, high fidelity polymerases may be used going forward. Optionally, isothermal amplification, for example using Phi29 DNA polymerase, can be used.

Following single-sided PCR, a MinElute PCR purification kit was used to isolate the single-sided PCR product. Optionally, rSAP enzymatic cleanup, a bead based cleanup or other column can be used to isolate the PCR product at this point.

Part 4—Tailing

The single-sided PCR product was polyadenylated (A-tailed) using a Terminal Transferase. Optionally, a polyG tail can be used, and is less variable with respect to the concentration of the DNA input.

Following polyadenylation, a MinElute PCR purification kit was used to isolate the A-tailed DNA. Optionally, rSAP enzymatic cleanup, a bead based cleanup or other column can be used to isolate the tailed DNA at this point.

Part 5—Adapter 2 Addition

The tailed PCR product was then used as a template in a second single-sided PCR (i.e., only one primer) that allowed the second adapter+primer to anneal to the poly-A tail and extend the full length of the molecule, thus including the adapter on the other side of the PCR product. Initially, this step was carried out with Taq polymerase. However, high fidelity polymerases may be used going forward. Optionally, isothermal amplification, for example using Phi29 DNA polymerase, can be used.

Following the second single-sided PCR reaction, a MinElute PCR purification kit was used to isolate the A-tailed DNA. Optionally, a bead based cleanup or other column can be used to isolate the PCR product at this point.

The PCR product was then checked by qPCR. Successful qPCR amplification indicated that a sequenceable library had been made.

Part 6—Indexing PCR

A standard indexing PCR reaction was used to add barcodes to adapters, followed by Kapa bead purification

Part 7—Sequencing

Standard high throughput sequencing methods were used to sequence the library. Optionally a one tube reaction (i.e., all enzymatic clean ups until the indexing, combining steps potentially poly-G tailing then heat inactivating and adding Adapter 2) can be used. An additional variation of the protocol is the adapter 1 addition, followed poly-G tailing, then adapter 2 addition and finally indexing PCR (no blunt or blocking).

Detailed Protocol

The following samples were processed according to the protocol set forth below.
(1) Negative control (water, called “Negative”), the 3′ end was not blocked
(2) 64 bp DNA digested into 2 parts by MseI to test blocking efficiency (called “Positive”), the 3′ end was not blocked
(3) 64 bp DNA digested into 2 parts by MseI to test blocking efficiency (called “Test”), the 3′ end was blocked.
   Unless otherwise indicated, sample PCR products, rSAP products/DNA, Klenow products were treated the same during processing.

Detailed Protocol

Part 1—Blunt Ending

The blunt ending was carried out using the conditions shown in Table 2 below:

TABLE 2
Blunt ending

| | Per Sample | Initial | | final | |
|---|---|---|---|---|---|
| Reagent | (ul) | concentration | | concentration | |
| T4 DNA | 2.0 | 3 | U/ul | 0.12 | U/ul |
| Polymerase | | | | | |
| Cutsmart Buffer | 0.40 | 10x | | 1x | |
| dNTPs | 1.60 | 10 | mM each | 48.5 | uM each |
| PCR product | 29.0 | 26.8 | ng/ul | 723 | ng total |
| Water | 0.00 | — | | — | |
| Sum | 33 | | | | |

1 Unit (U) T4 DNA polymerase per g DNA was used. PCR product was from the NL01 SNP PCR, and was MseJ digested. The reaction was incubated at 12° C. for 15 minutes, and then at 75 (C for 20 minutes. A Qiaquick PCR purification kit was used to remove nucleotides from 33 μL to 65 μL of the reaction mixture.

Part 2—Blocking

The blunt ended PCR product was blocked using the conditions shown in Tables 2-5 below:

TABLE 3
Sample 1: Klenow Negative Control (with water) - No tail

| | Per Sample | Initial | | final | |
|---|---|---|---|---|---|
| Reagent | (ul) | concentration | | concentration | |
| Klenow (exo-) | 3 | 5 | U/ul | 0.3 | U/ul |
| Cutsmart Buffer | 5 | 10x | | 1x | |
| dNTPs | 2.5 | 10 | mM each | 500 | uM each |
| Water (no DNA) | 30 | — | | — | |
| Water | 9.5 | — | | — | |
| Sum | 50 | | | | |

TABLE 4
Sample 2: Klenow Positive Control (with DNA + dNTPS) - Tail

| | Per Sample | Initial | | final | |
|---|---|---|---|---|---|
| Reagent | (ul) | concentration | | concentration | |
| Klenow (exo-) | 3 | 5 | U/ul | 0.3 | U/ul |
| Cutsmart Buffer | 5 | 10x | | 1x | |
| dNTPs | 2.5 | 10 | mM each | 500 | uM each |
| rSAP product | 30 | 13 | ng/ul | 5.2 | ng/ul |
| Water | 9.5 | — | | — | |
| Sum | 50 | | | | |

TABLE 5
Sample 3: Klenow Test (with DNA + ddNTPs) - Testing

| | Per Sample | Initial | | final | |
|---|---|---|---|---|---|
| Reagent | (ul) | concentration | | concentration | |
| Klenow (exo-) | 3 | 5 | U/ul | 0.3 | U/ul |
| Cutsmart Buffer | 5 | 10x | | 1x | |
| ddNTPs | 0.5 | 2.5 | mM each | 500 | uM each |
| rSAP product | 30 | 13 | ng/ul | 5.2 | ng/ul |
| Water | 11.5 | — | | — | |
| Sum | 50 | | | | |

All samples were incubated for 40 minutes at 37° C., and then for 75° C. for 20 minutes. Excess nucleotides were then removed using the Qiaquick Nucleotide removal kit, and eluted into 50 μL elution buffer (EB).

Part 3—Adapter 1

Single-sided Adapter 1 PCR was carried out using the following reaction conditions:

TABLE 6
Adapter 1 PCR Reaction Mixture

| | Per Sample | Initial | final |
|---|---|---|---|
| Reagent | (ul) | concentration | concentration |
| Taq 2X MM | 110.5 | 2X | 1X |
| NL01_Rev + Adapter | 4.4 | 10 uM | 0.2 uM |
| Klenow product | 20 | | |
| Water | 86.08 | — | — |
| Sum | 221 | | |

The primer was designed to target a phenotypic SNP present in the PCR product, and also had an NEBNext Adapter attached.

TABLE 7
Adapter 1 PCR Reaction Conditions

| Run for: | |
|---|---|
| 95° C. for 3 min | |
| 95° C. for 30 sec | 45 cycles |
| 68° C. for 60 sec | |
| 68° C. for 5 min | |
| 12° C. hold | |

Other, higher fidelity polymerases, for example the Qiagen high fidelity polymerase master mix (MM), may also be suitable. It may also be possible to vary the number of cycles (i.e., use more than 45 or less than 45 cycles). Following single-sided PCR, the MinElute PCR purification kit was used to purify the PCR product. This removed unincorporated nucleotides and small un-extended fragments. 221 μL PCR product were eluted into 60 μL EB.

Part 4—A-Tailing

PCR products were polyadenylated using the following reaction conditions:

TABLE 8
Polyadenylation Reaction

| | Per Sample | Initial | | final | |
|---|---|---|---|---|---|
| Reagent | (ul) | concentration | | concentration | |
| Tdt buffer | 7.5 | 10x | | 1x | |
| CoCl2 Solution | 7.5 | 2.5 | mM | 0.25 | mM |
| dATP | 2.7 | 1 | mM | 2,737 | |
| Terminal transferase | 0.8 | 20 | U/ul | 0.2 | U/ul |
| DNA | 50 | 1.37 | | pmol | |
| Water | 6.5 | — | | — | |
| Sum | 75 | | | | |

For dATP, 1:1000 pmol ends to pmol dNTPs was used. 0.2 U/μL Terminal Transferase for up to 5 pmol were used. 52 ng of DNA were used for the Test and Negative samples, 101 ng DNA was used for the Positive sample. Reactions were incubated at 37° C. for 30 minutes, and then at 70° C. for 10 minutes. A MinElute Reaction cleanup kit was used to purify polyadenylated PCR products. 75 μL of polyadenylated PCR product were eluted into 40 μL of EB.

Part 5—Adapter 2 Addition

The second adapter was added using the following PCR conditions:

TABLE 9
Adapter 2 PCR Reaction Mixture

| | Per Sample | Initial | final |
|---|---|---|---|
| Reagent | (ul) | concentration | concentration |
| Taq 2X MM | 100 | 2X | 1X |
| P7_PolyT_Adapter | 4.0 | 10 uM | 0.2 uM |
| DNA | 35 | | |
| Water | 61 | — | — |
| Sum | 200 | | |

The second primer was designed to have a polyT sequence with an NEBNext adapter sequence attached.

TABLE 10
Adapter 2 PCR Reaction Conditions

| Run for: | |
|---|---|
| 95° C. for 3 min | |
| 95° C. for 30 sec | 45 cycles |
| 60° C. for 60 sec | |
| 68° C. for 60 sec | |
| 12° C. hold | |

Other, higher fidelity polymerases, for example the Qiagen high fidelity polymerase master mix (MM), may also be suitable. It may also be possible to vary the number of cycles (i.e., use more than 45 or less than 45 cycles). A MinElute Reaction cleanup kit was used to purify polyAdenylated PCR products. 200 μL PCR product were eluted into 30 μL of EB. The PCR product was checked by qPCR amplification. Successful amplification indicated a sequenceable library had been made.

Part 6—Indexing PCR (iPCR1)

Indexing PCR to add barcodes to the library was carried out as follows:

TABLE 11
Indexing PCR Reaction Mixture

| | Per Sample | | Initial | | final | |
|---|---|---|---|---|---|---|
| Reagent | (ul) | x3 | concentration | | concentration | |
| Kapa HiFi Buffer | 5.00 | 15 | 5X | | 1X | |
| Kapa dNTP mix | 0.75 | 2.25 | 10 | mM each | 0.3 | mM each |
| Kapa HiFi Polym | 0.50 | 1.5 | 1 | U/ul | 0.5 | U total |
| Fwd (i5) | 0.75 | 2.3 | 10 | uM | 0.3 | uM |
| Rev (i7) | 0.75 | 2.3 | 10 | uM | 0.3 | uM |
| Water | 17.25 | 51.75 | — | | — | |
| Sum | 25 | 25 | | | | |

NEBNext indexes that amplify only NEBNext adapters were used on the indexing primers. 5 μL DNA (post Adapter 2 addition) was added.

TABLE 12
Indexing PCR Reaction Conditions

| Run for: | |
|---|---|
| 95° C. for 3 min | |
| 98° C. for 20 sec | 6 Cycles* |
| 60° C. for 15 sec | |
| 72° C. for 20 sec | |
| 72° C. for 3 min | |
| 12° C. hold | |

*The number of cycles was calculated based off of qPCR plateau values.

Following indexing PCR, Kapa bead purification was used to purify the PCR product. 25 μL of PCR product was eluted into 25 μL EB.

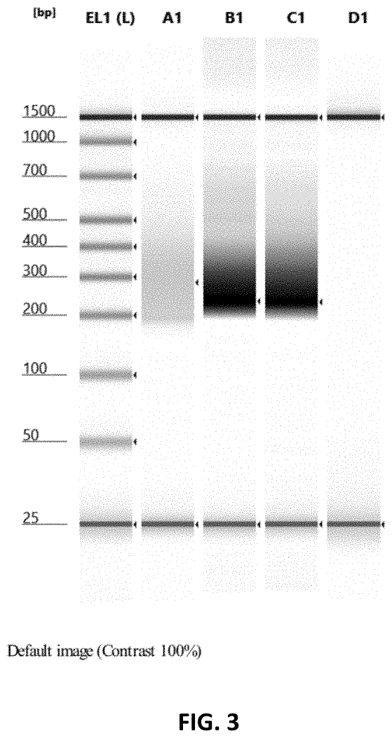

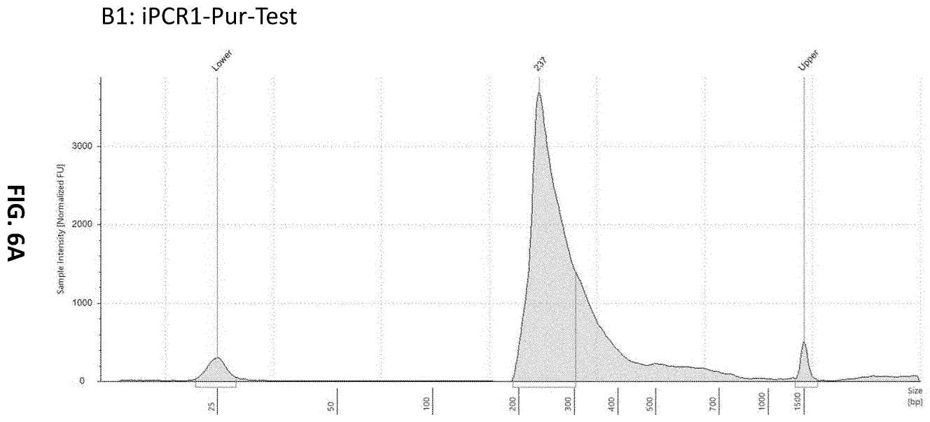

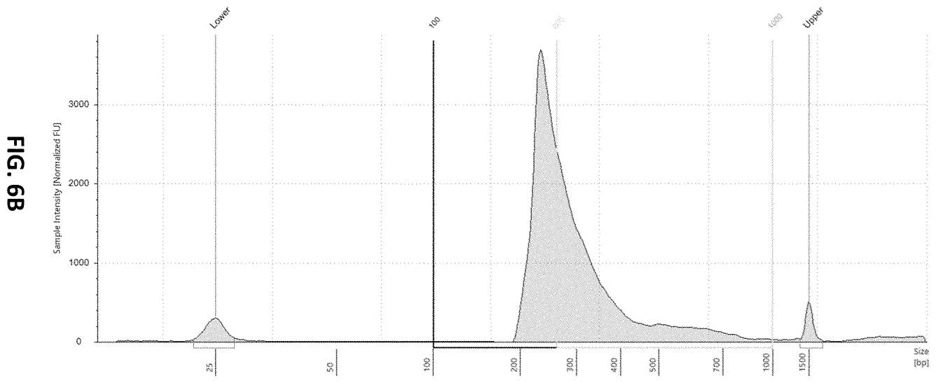

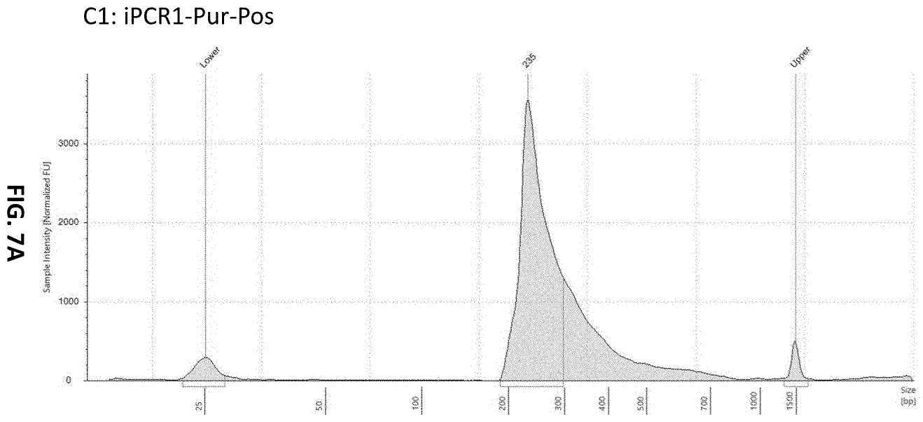

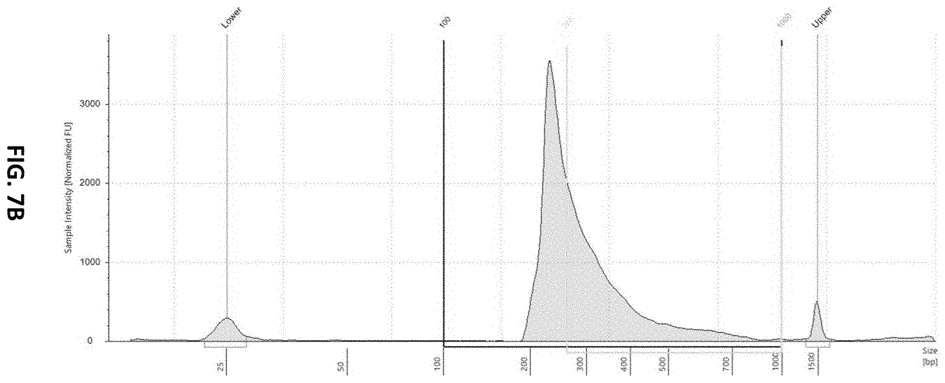

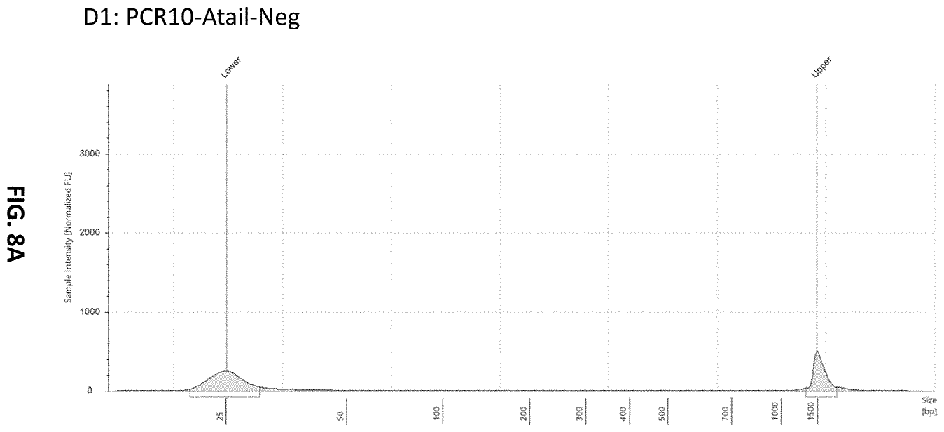

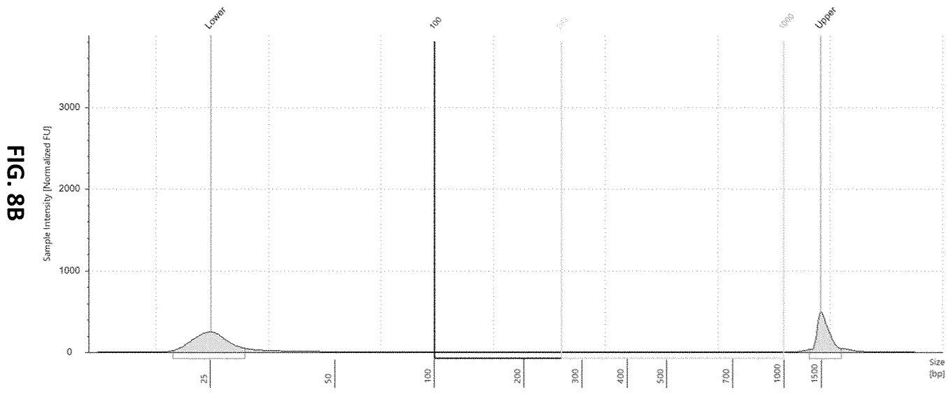

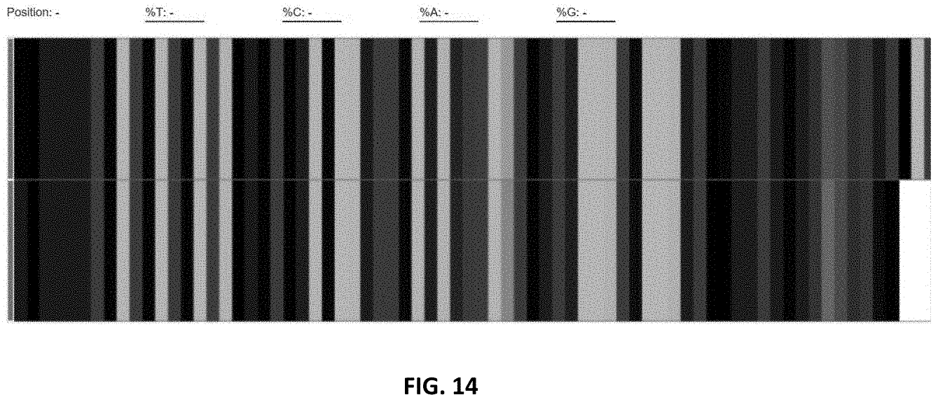

The Positive, Negative and Test sample libraries created with this protocol, as well as an A-tail negative control, were quantified using the Agilent High Sensitivity D1000 ScreenTape System following indexing PCR and purification, and the results are shown in FIGS. 3-9 below. See Table 13 below for sample/well identity and concentration, and Tables 14-22 for quantification corresponding to FIGS. 14-8.

TABLE 13
Sample Information

| | | | | |
|---|---|---|---|---|
| Well | Concentration (pg/μL) | Sample Description | Alert | Observations |

| | | | |
|---|---|---|---|
| EL1 | 2350 | Electronic Ladder | Ladder |
| A1 | 124 | iPCR1-Pur-Neg | |
| B1 | 7140 | iPCR1-Pur-Test | |
| C1 | 6380 | iPCR1-Pur-Pos | |
| D1 | | PCR10-Atail-Neg | |

Neg = Negative (sample 1), Test = Test (sample 3), Pos = Positive (sample 2), Atail-Neg = Atailing negative control.

TABLE 14
Electronic Ladder Peak Table

| | Calibrated | Assigned | Peak | | | |
|---|---|---|---|---|---|---|
| Size | Conc. | Conc. | Molarity | % Integrated | Peak | |
| [bp] | [pg/μl] | [pg/μl] | [pmol/l] | Area | Comment | Observations |
| 25 | 340 | — | 20900 | — | | Lower Marker |
| 50 | 265 | — | 8160 | 11.28 | | |
| 100 | 278 | — | 4270 | 11.82 | | |
| 200 | 290 | — | 2230 | 12.32 | | |
| 300 | 304 | — | 1560 | 12.95 | | |
| 400 | 306 | — | 1180 | 13.00 | | |
| 500 | 312 | — | 961 | 13.29 | | |
| 700 | 286 | — | 629 | 12.19 | | |
| 1000 | 309 | — | 476 | 13.15 | | |
| 1500 | 250 | 250 | 256 | — | | Upper Marker |

TABLE 15
iPCR1-Pur-Neg Peak Table

| | Calibrated | Assigned | Peak | | | |
|---|---|---|---|---|---|---|
| Size | Conc. | Conc. | Molarity | % Integrated | Peak | |
| [bp] | [pg/μl] | [pg/μl] | [pmol/l] | Area | Comment | Observations |
| 25 | 425 | — | 26200 | — | | Lower Marker |
| 286 | 124 | — | 665 | 100.00 | | |
| 1500 | 250 | 250 | 256 | — | | Upper Marker |

TABLE 16
iPCR1-Pur-Neg Region Table

| | | | | Region | | | |
|---|---|---|---|---|---|---|---|
| From | To | Average | Conc. | Molarity | % of | Region | |
| [bp] | [bp] | Size [bp] | [pg/μl] | [pmol/l] | Total | Comment | Color |
| 100 | 1000 | 331 | 1840 | 9560 | 96.75 | | Dark |
| 265 | 1000 | 387 | 1230 | 5240 | 64.55 | | Light |

TABLE 17
iPCR1-Pur-Test Peak Table

| | Calibrated | Assigned | Peak | | | |
|---|---|---|---|---|---|---|
| Size | Conc. | Conc. | Molarity | % Integrated | Peak | |
| [bp] | [pg/μl] | [pg/μl] | [pmol/l] | Area | Comment | Observations |
| 25 | 383 | — | 23600 | — | | Lower Marker |
| 237 | 7140 | — | 46400 | 100.00 | | |
| 1500 | 250 | 250 | 256 | — | | Upper Marker |

TABLE 18
iPCR1-Pur-Test Region Table

| | | | | Region | | | |
|---|---|---|---|---|---|---|---|
| From | To | Average | Conc. | Molarity | % of | Region | |
| [bp] | [bp] | Size [bp] | [pg/μl] | [pmol/l] | Total | Comment | Color |
| 100 | 1000 | 309 | 10400 | 57000 | 97.05 | | Dark |
| 265 | 1000 | 373 | 5540 | 25100 | 51.50 | | Light |

TABLE 19
iPCR1-Pur-Pos Peak Table

| | Calibrated | Assigned | Peak | | | |
|---|---|---|---|---|---|---|
| Size | Conc. | Conc. | Molarity | % Integrated | Peak | |
| [bp] | [pg/μl] | [pg/μl] | [pmol/l] | Area | Comment | Observations |
| 25 | 404 | — | 24900 | — | | Lower Marker |
| 235 | 6380 | — | 41900 | 100.00 | | |
| 1500 | 250 | 250 | 256 | — | | Upper Marker |

TABLE 20
iPCR1-Pur-Pos Region Table

| | | | | Region | | | |
|---|---|---|---|---|---|---|---|
| From | To | Average | Conc. | Molarity | % of | Region | |
| [bp] | [bp] | Size [bp] | [pg/μl] | [pmol/l] | Total | Comment | Color |
| 100 | 1000 | 305 | 9660 | 53100 | 97.32 | | Dark |
| 265 | 1000 | 367 | 5100 | 23200 | 51.31 | | Light |

TABLE 21
PCR10-Atail-Neg Peak Table

| | Calibrated | Assigned | Peak | | | |
|---|---|---|---|---|---|---|
| Size | Conc. | Conc. | Molarity | % Integrated | Peak | |
| [bp] | [pg/μl] | [pg/μl] | [pmol/l] | Area | Comment | Observations |
| 25 | 376 | — | 23200 | — | | Lower Marker |
| 1500 | 250 | 250 | 256 | — | | Upper Marker |

TABLE 22
PCR10- Atail-Neg Region Table

| | | | | Region | | | |
|---|---|---|---|---|---|---|---|
| From | To | Average | Conc. | Molarity | % of | Region | |
| [bp] | [bp] | Size [bp] | [pg/μl] | [pmol/l] | Total | Comment | Color |
| 100 | 1000 | 440 | 5.59 | 45.5 | 5.13 | | Dark |
| 265 | 1000 | 642 | 3.13 | 12.6 | 2.88 | | Light |

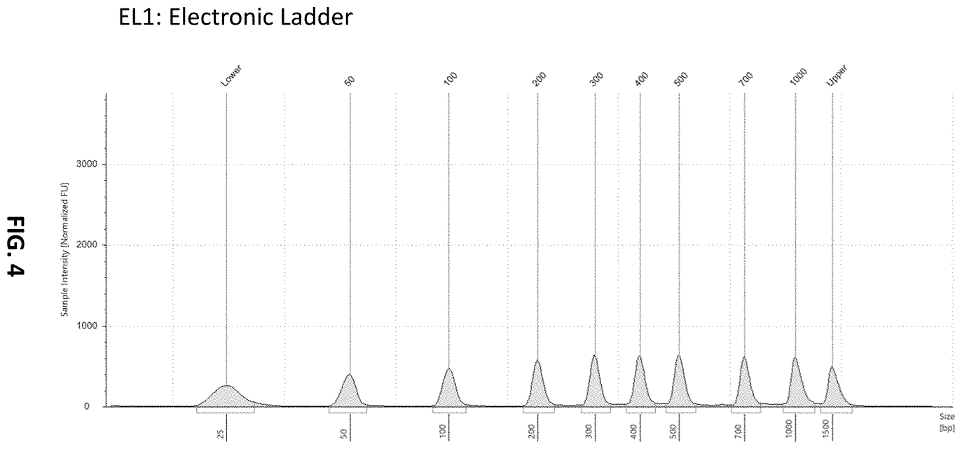

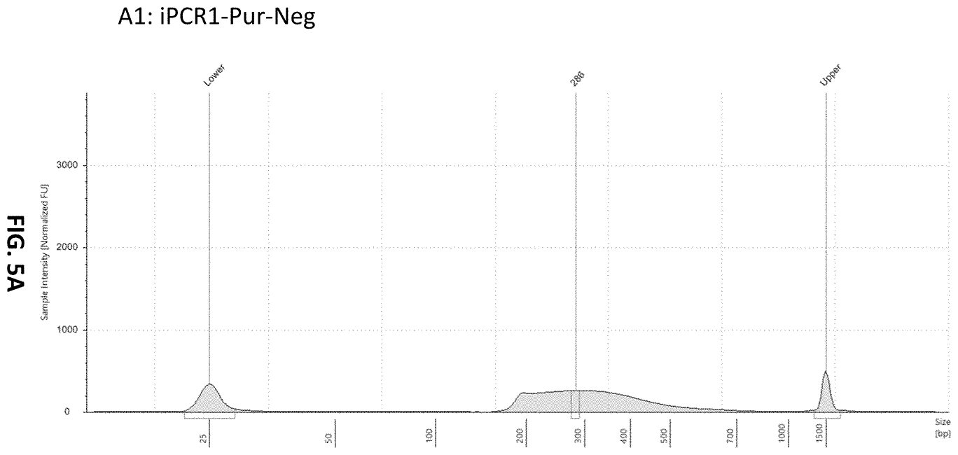

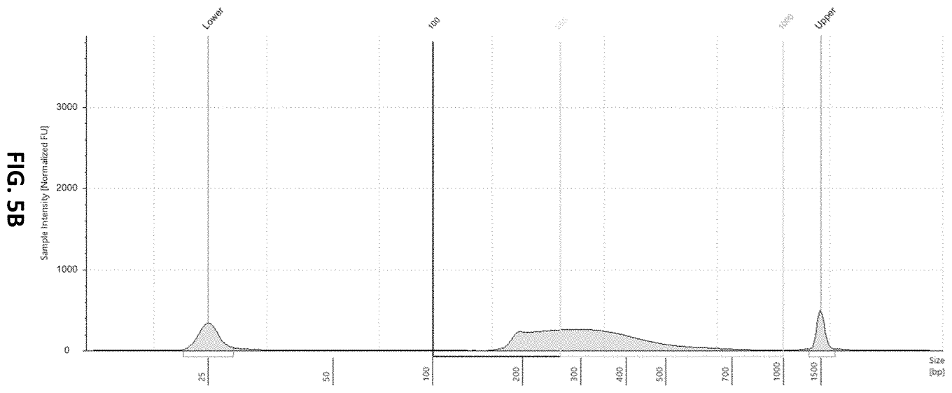

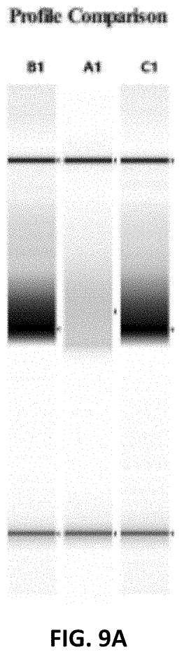

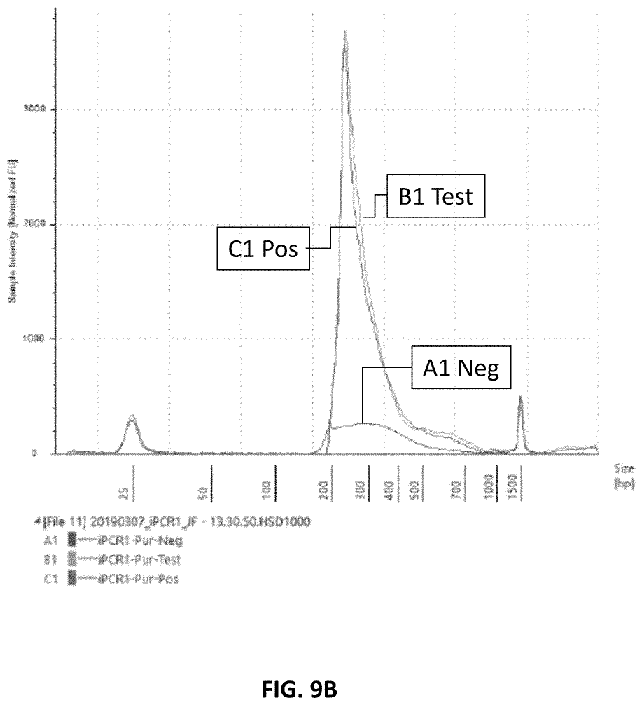

FIG. 3 shows a picture of the gel. FIG. 4 shows the ladder, while FIGS. 5A-5B, FIGS. 6A-6B, FIGS. 7A-7B and FIGS. 8A-8B show High Sensitivity D1000 ScreenTape results for the Negative, Test, Positive and Atail negative control samples, respectively. FIG. 9A and FIG. 9B show a comparison of the Positive, Negative and Test libraries.

Once purified, the Positive and Test libraries were high throughput sequenced.

### Example 2: Library Analysis

FastQC analysis was done on the trimmed, complexity and quality filtered data from Run 2 of both samples (Positive and Test). Analysis of the high throughput dataset was carried out using Samtools and FastQC, and the data summarized using MultiQC. Table 23 shows an overview of the general statistics from the two libraries.

TABLE 22
General Statistics

| Sample | Reads Mapped | % Duplicate | Average | Total Sequences |
|---|---|---|---|---|
| Name | (millions) | Reads | % GC | (Millions) |
| Positive | 1 | 95% | 49% | 1 |
| Test | 0.3 | 95.40% | 49% | 0.3 |

Table 25 shows the output from the Samtools flagstat function, which does a full pass through the input file and calculates and prints the statistics. Results are in Millions of reads.

TABLE 24
Samtools Flagstat Output

| Parameter | Test | Positive |
|---|---|---|
| Total Reads | 0.27M | 0.96M |
| Total Passed QC | 0.27M | 0.96M |
| Mapped | 0.27M | 0.96M |
| Duplicates | 0.0M | 0.0M |
| Paired in Sequencing | 0.0M | 0.0M |
| Properly Paired | 0.0M | 0.0M |
| Self mate mapped | 0.0M | 0.0M |
| Singletons | 0.0M | 0.0M |
| Mapped to different | 0.0M | 0.0M |
| chromosome | | |
| Diff chr (MapQ >= 5) | 0.0M | 0.0M |

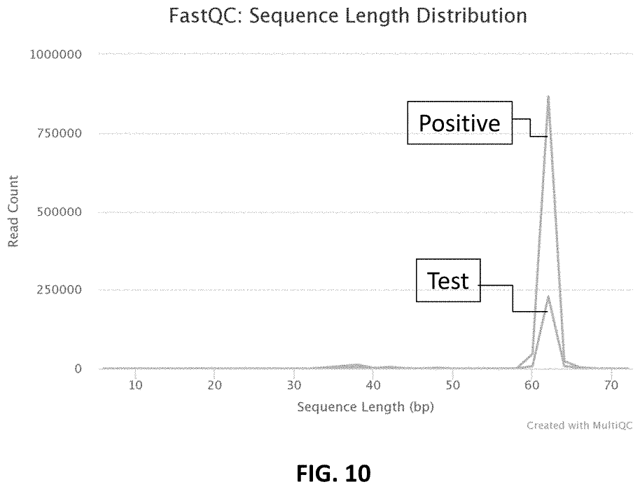

The sequencing showed that mainly the full-length 64 bp product was successfully sequenced, rather than the blocked, shorter fragments (this can be seen from the fragment size distribution shown in FIG. 10). Hence, it may be possible to omit the blocking and blunting steps.

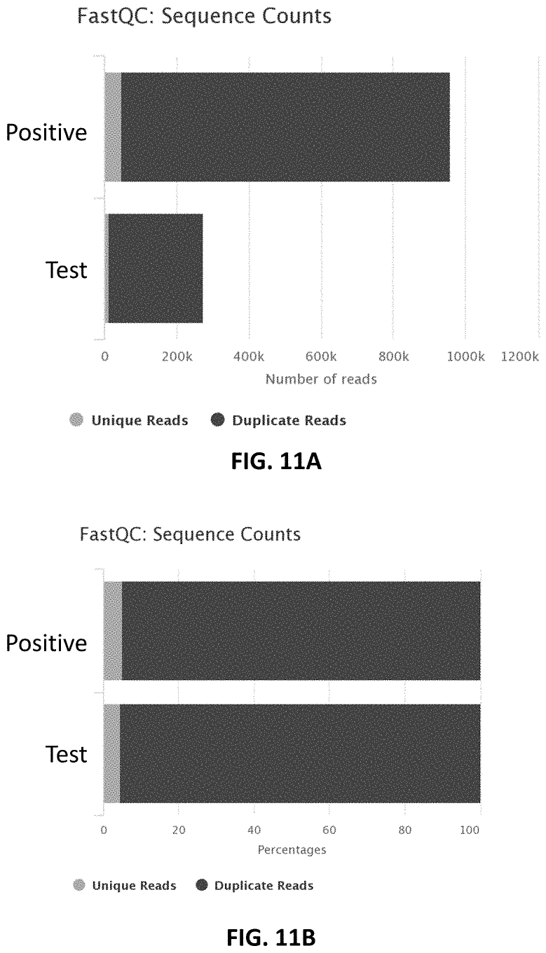

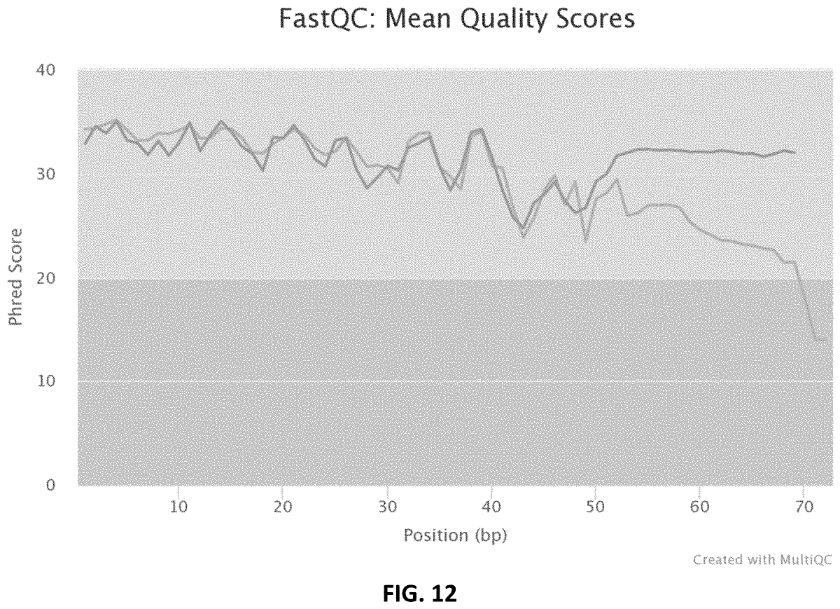

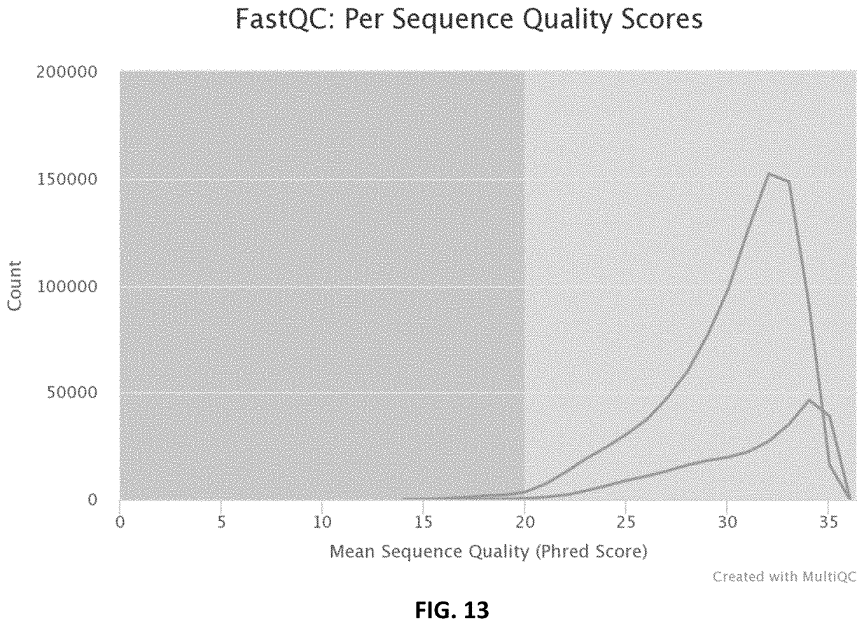

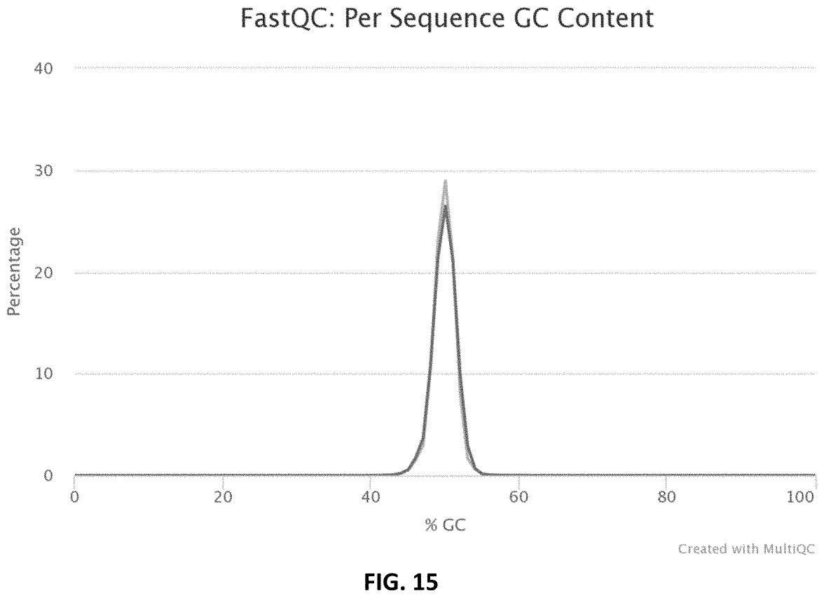

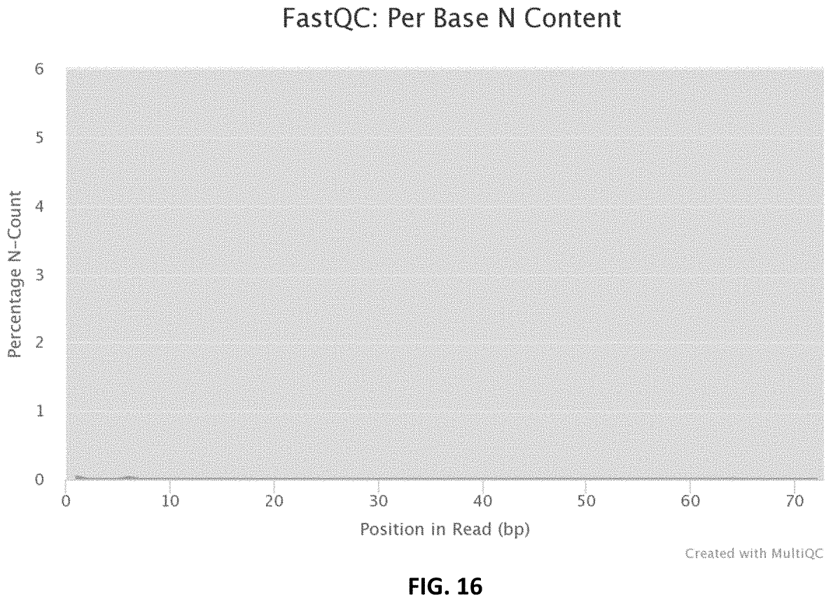

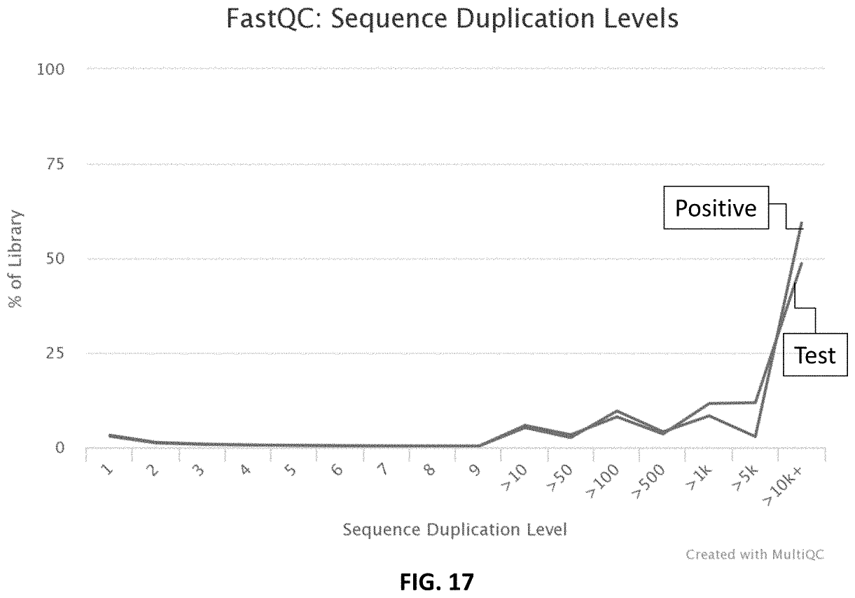

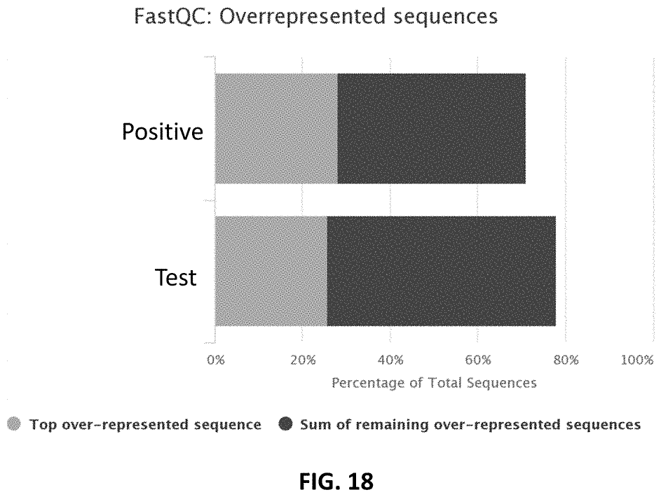

The samples went on two runs since the first did not produce enough data. In the first run, the Positive sample produced 74 reads. In the second run, the Positive sample produced 1,095,378 reads. 957,262 of these reads (87%) mapped sufficiently to the expected sequence. In the first run, the Test sample produced 385 reads. In the second run, the Test sample produced 289,368 reads. 272,245 of these reads (94%) mapped sufficiently to the expected sequence. No statistics are provided for the Run 1, since the read count was so low that the results are likely to just be sporadic. Statistics for Run 2 are presented in FIG. 10, FIG. 11A, FIG. 11B, FIG. 12, FIG. 13, FIG. 14, FIG. 15, FIG. 16, FIG. 17 and FIG. 18.

### Example 3: Phi29 DNA Polymerase-Based Protocol for Ligation-Free Library Preparation Using polyG Tailing Followed by Random Priming

This protocol uses terminal transferase to add a polyG tail to a mixture of nucleic acids in a sample, followed by addition of a first adapter using Phi29 DNA polymerase with a complementary polyC sequence, and a second adapter using Phi29 DNA polymerase with a 3′ random priming sequence.

Addition of the polyG Tail:

Addition of the polyG tail can potentially be carried out in an emulsion.

The polyG tail is added to DNA fragments in a sample using the reaction mixture shown in Table 25 below:

TABLE 25
polyG Tailing Reaction Mixture

| | Per Sample | Initial | | final | |
|---|---|---|---|---|---|
| Reagent | (μL) | concentration | | concentration | |
| Tdt buffer | 2.5 | 10x | | 1x | |
| CoCl₂ | 2.5 | 2.5 | mM | 0.25 | mM |
| Solution | | | | | |
| dGTP | 0.12 | 1 | mM | 1:1000 ratio | |
| Terminal | 0.3 | 20 | U/ul | 0.2 | U/ul |

| | | | | |
|---|---|---|---|---|
| transferase | | | | |
| DNA | 5.18 | 10 | ng total | — |

| | | | |
|---|---|---|---|
| Water | 14.45 | — | — |
| Sum | 25.00 | | |

The amount of dGTP nucleotides is determined from quality control (QC) measurements of the sample DNA, i.e. length and concentration, which are used to calculate the ratio of dGTPs to 3′ ends in the sample. The value provided here is for one particular sample. The volume of DNA added will depend on concentration. Length and concentration of DNA fragments in a sample can be measured with an Agilent Bioanalyzer, e.g. This step may become a standardized amount (e.g. if the concentration of the DNA samples are between X and Y, use Z, if between A and B, use C).

Incubate G-tailing reactions at 37° C. for 30 minutes, then at 70° C. for 10 minutes.

Following G-tailing, reactions are purified using SPRI magnetic beads at 1.8×, and eluted in 15 μL.

Attachment of a First Adapter:

Attachment of a first adapter can potentially be carried out in an emulsion.

A first adapter with a sequence of 5′-GACTGGAGTTCAGACGTGTGCTCTTCCGATCCCCCCC-3′ (Ad2) (SEQ ID NO:24) is attached to the polyG-tailed fragments in the DNA sample using the conditions below. Phosphorothioates can be incorporated into the Ad2 adapter to avoid degradation by Phi29's exonuclease activity. For example, an adapter of sequence 5′-G*A*CTGGAGTTCAGACGTGTGCTCTTCCGATCCCCC*C*C-3′ (SEQ ID NO:25), where the (*) represent Phosphorothioates can be used. 3′ C tails of other lengths are also possible, e.g. 5C, 9C etc. In some cases, 7Cs are used.

TABLE 26
Reaction Mixture for Attachment of a First Adapter

| | Per Sample | Initial | final |
|---|---|---|---|
| Reagent | (μl) | concentration | concentration |
| Phi Buffer | 2.50 | 10x | 1x |
| Ad2 | 0.50 | 10 uM | 0.2 uM |
| DNA | 10.00 | — | |
| Water | 10.00 | — | — |
| Sum | 22.35 | | |

Note, the buffer will change if other polymerases are used for this step. DNA input can also vary.

Rampdown: incubate samples from Table X2 at 98° C. for 5 minutes, then incubate at 65° C., decreasing in 5 degree intervals to 4° C., for 30 seconds each. Alternatively, samples can be heated to 95° C. for 5 minutes, and then cooled on ice for 2 minutes.

Following the Rampdown, add to the reactions from Table 25 the following:

TABLE 26
Reaction Mixture for Attachment of
a First Adapter Following Rampdown

| | Per Sample | (Final | |
|---|---|---|---|
| Reagent | (μl) | Concentration) | |
| dNTPs | 0.63 | (0.25 | nM) |
| BSA | 0.025 | (0.1%) | |
| Phi29 | 2.00 | (20 | Units) |

| | |
|---|---|
| 25.00 | — |

Other polymerases or combinations of multiple polymerases have been considered, e.g. Klenow exo- or Bsu DNA Polymerase, Large Fragment (BSU). Also, other concentrations of Phi29 may also be used.

Samples to which dNTPs, BSA and Phi29 have been added as shown in Table X3 are incubated at 30° C. for 60 minutes, followed by incubation at 65° C. for 10 minutes.

Following incubation, reactions are purified using SPRI magnetic beads at 1.8×, and eluted in 15 μL. This step is potentially optional.

Attachment of a Second Adapter:

A second adapter with a sequence of 5′-ACACTCTTTCCCTACACGACGCTCTTCCGATCNNNNNNNNN-3′ (Ad1) (SEQ ID NO:26) is attached to the fragments in the DNA sample using the conditions below. Phosphorothioates can be incorporated into the Ad1 adapter to avoid degradation by Phi29's exonuclease activity. For example, an adapter of sequence e.g. 5′-A*C*ACTCTTTCCCTACACGACGCTCTTCCGATCNNNNNNN*N*N-3′ (SEQ ID NO:27), where the (*) represent Phosphorothioates can be used. The N tails of the adapter can also have variable length (e.g. 6Ns, 9Ns, 16Ns, etc.). In some examples, 9Ns are used.

TABLE 27
Reaction Mixture to Attach Second Adapter

| | Per Sample | Initial | Final |
|---|---|---|---|
| Reagent | (μL) | concentration | concentration |
| Phi Buffer | 2.50 | 10x | 1x |
| Ad1 | 0.50 | 10 uM | 0.2 uM |
| DNA | 10.00 | — | |
| Water | 10.00 | — | — |
| Sum | 22.35 | | |

Note, the buffer will change if a different polymerase is used. DNA input can also vary.

Rampdown: incubate reactions from Table X4 at 98° C. for 5 minutes, then incubate the reactions at 65° C., decreasing in 5 degree intervals to 4° C., for 30 seconds each. Alternatively, samples can be heated to 95° C. for 5 minutes, and cooled on ice for 2 minutes.

Following the Rampdown, add to the reactions from Table 27 the following:

TABLE 28
Reaction Mixture for Attachment of
a Second Adapter Following Rampdown

| | Per Sample | (Final | |
|---|---|---|---|
| | (μL) | Concentration) | |
| dNTPs | 0.63 | (0.25 | nM) |
| BSA | 0.025 | (0.1%) | |
| Phi29 | 2.00 | (20 | Units) |

25.00

Other polymerases or combinations of multiple polymerases have been considered, e.g. Klenow exo- or Bsu DNA Polymerase, Large Fragment (BSU). Also, other concentrations of Phi29 may also be used.

Samples to which dNTPs, BSA and Phi29 have been added as shown in Table X5 are incubated at 30° C. for 60 minutes, followed by incubation at 65° C. for 10 minutes.

Following incubation, reactions are purified using SPRI magnetic beads at 1.8×, and eluted in 15 μL. This step is potentially optional.

Index PCR Reaction:

After addition of the first and second adapters, samples are indexed for sequencing using the following conditions.

TABLE 29
Reaction Mixture for Indexing PCR Reaction

| | Per Sample | Initial | Final |
|---|---|---|---|
| Reagent | (μL) | concentration | concentration |
| Kapa HiFi MM | 12.50 | 2x | 1x |
| Water | 0.50 | — | — |
| Sum | Add 13 ul to | | |
| | each reaction | | |
| DNA | 10.00 | — | <−add individually |
| i5 primer | 1.00 | 5 μM | <−add individually |
| i7 primer | 1.00 | 5 μM | <−add individually |

TABLE 30
Indexing PCR
Temperature and Time

| | |
|---|---|
| 98° C. for 2 min | |
| 98° C. for 25 sec | 20 cycles |
| 60° C. for 20 sec | |
| 72° C. for 20 sec | |
| 72° C. for 2 min | |
| 12° C. hold | |

Note that the number of cycles can be varied from 20, for example based on the starting concentration.

Following indexing PCR, reactions are purified using SPRI magnetic beads at 1.8×, and eluted in 25 μL.

Samples are ready for high throughput sequencing, following quality control to determine concentration and size of DNA fragments, and purity.

### Example 4: Phi29 DNA Polymerase-Based Protocol for Ligation-Free Library Preparation Using Random Priming Followed by polyG Tailing

This protocol uses a Phi29 DNA polymerase to add a first adapter to DNA fragments in a sample using random priming, followed by polyG tailing by terminal transferase and addition of a second adapter using Phi29 DNA polymerase with a complementary polyC sequence.

Reaction volumes given are for 13.5 samples (13 samples, plus an extra 0.5 to allow for pipetting error).

Attachment of a First Adapter:

Attachment of a first adapter can potentially be carried out in an emulsion.

A first adapter with a sequence of 5′-ACACTCTTTCCCTACACGACGCTCTTCCGATCNNNNNNNNN-3′ (Ad1) (SEQ ID NO:26) is attached to the fragments in the DNA sample using the conditions below. Phosphorothioates can be incorporated into the Ad1 adapter to avoid degradation by Phi29's exonuclease activity. For example, an adapter of sequence e.g. 5′-A*C*ACTCTTTCCCTACACGACGCTCTTCCGATCNNNNNNN*N*N-3′ (SEQ ID NO:27), where the (*) represent Phosphorothioates can be used. The N tails of the adapter can also have variable length (e.g. 6Ns, 9Ns, 16Ns, etc.). In some cases, 9Ns are used.

TABLE 31
Reaction Mixture for Attachment of a First Adapter

| | Per Sample | Initial | | Final |
|---|---|---|---|---|
| Reagent | (μL) | concentration | | concentration |
| Phi Buffer | 2.50 | 10x | | 1x |
| Ad1 | 0.50 | 10 | uM | 0.2 uM |
| DNA | 5.18 | 10 | ng total | |

| | | | |
|---|---|---|---|
| Water | 14.82 | — | — |
| Sum | 22.35 | | |

Note, the buffer will change if other polymerases are used for this step. DNA input can also vary, and volume will vary with concentration.

Rampdown: incubate samples from Table X7 at 98° C. for 5 minutes, then incubate at 65° C., decreasing in 5 degree intervals to 4° C., for 30 seconds each. Alternatively, samples can be heated to 95° C. for 5 minutes, and then cooled on ice for 2 minutes.

Following the Rampdown, add to the reactions from Table 31 the following:

TABLE 32
Reaction Mixture for Attachment of
a First Adapter Following Rampdown

| | Per Sample |
|---|---|
| | (μL) |
| dNTPs | 0.63 |
| BSA | 0.025 |
| Phi29 | 2.00 |
| | 25.00 |

Other polymerases or combinations of multiple polymerases have been considered, e.g. Klenow exo- or Bsu DNA Polymerase, Large Fragment (BSU). Also, other concentrations of Phi29 may also be used.

Samples to which dNTPs, BSA and Phi29 have been added as shown in Table X8 are incubated at 30° C. for 60 minutes, followed by incubation at 65° C. for 10 minutes.

Following incubation, reactions are purified using SPRI magnetic beads at 1.8×, and eluted in 15 μL. This step is potentially optional.

Addition of the polyG Tail:

Following addition of the first adapter, a polyG tail is added to DNA fragments in a sample using the reaction mixture shown in Table 33 below:

TABLE 33
polyG Tailing Reaction Mixture

| | Per Sample | Initial | | Final | |
|---|---|---|---|---|---|
| Reagent | (μL) | concentration | | concentration | |
| Tdt buffer | 2.50 | 10x | | 1x | |
| CoCl2 Solution | 2.50 | 2.5 | mM | 0.25 | mM |
| dGTP | 0.088 | 1 | mM | 1:1000 | pmol ends |
| Terminal transferase | 0.25 | 20 | U/ul | 0.2 | U/ul |

| | | | |
|---|---|---|---|
| DNA | 13.00 | — | — |
| Water | 6.66 | — | — |
| Sum | 25.00 | | |

The amount of dGTP nucleotides is determined from quality control (QC) measurements of the sample DNA, i.e. length and concentration, which are used to calculate the ratio of dGTPs to 3′ ends in the sample. The value provided here is for one particular sample. The volume of DNA added will also change based on concentration. Length and concentration of DNA fragments in a sample can be measured with an Agilent Bioanalyzer, e.g. This step may become a standardized amount (e.g. if the concentration of the DNA samples are between X and Y, use Z, if between A and B, use C).

Incubate G-tailing reactions at 37° C. for 30 minutes, then at 70° C. for 10 minutes.

Following G-tailing, reactions are purified using SPRI magnetic beads at 1.8×, and eluted in 15 μL.

Attachment of a Second Adapter:

A second adapter with a sequence of 5′-GACTGGAGTTCAGACGTGTGCTCTTCCGATCCCCCCC-3′ (Ad2) (SEQ ID NO:24) is attached to the polyG-tailed fragments in the DNA sample using the conditions below. Phosphorothioates can be incorporated into the Ad2 adapter to avoid degradation by Phi29's exonuclease activity. For example, an adapter of sequence 5′-G*A*CTGGAGTTCAGACGTGTGCTCTTCCGATCCCCC*C*C-3′ (SEQ ID NO:25), where the (*) represent Phosphorothioates can be used. 3′ C tails of other lengths are also possible (e.g. 5C, 9C, etc.). In some cases, 7Cs are used.

TABLE 34
Reaction Mixture for Attachment of a Second Adapter

| | Per Sample | Initial | Final |
|---|---|---|---|
| Reagent | (μL) | concentration | concentration |
| Phi Buffer | 2.50 | 10x | 1x |
| Ad2 | 0.50 | 10 uM | 0.2 uM |
| DNA | 10.00 | — | |
| Water | 10.00 | — | — |
| Sum | 22.35 | | |

Note, the buffer will change if other polymerases are used for this step. Note, DNA input can vary, and volume will vary with concentration.

Rampdown: incubate samples from Table X10 at 98° C. for 5 minutes, then incubate at 65° C., decreasing in 5 degree intervals to 4° C., for 30 seconds each. Alternatively, samples can be heated to 95° C. for 5 minutes, and then cooled on ice for 2 minutes.

Following the Rampdown, add to the reactions from Table 34 the following:

TABLE 35
Reaction Mixture for Attachment of
a Second Adapter Following Rampdown

| | Per Sample (ul) |
|---|---|
| dNTPs | 0.63 |
| BSA | 0.025 |
| Phi29 | 2.00 |
| | 25.00 |

Other polymerases or combinations of multiple polymerases have been considered, e.g. Klenow exo- or Bsu DNA Polymerase, Large Fragment (BSU). Also, other concentrations of Phi29 may also be used.

Samples to which dNTPs, BSA and Phi29 have been added as shown in Table X11 are incubated at 30° C. for 60 minutes, followed by incubation at 65° C. for 10 minutes.

Following incubation, reactions are purified using SPRI magnetic beads at 1.8×, and eluted in 15 μL. This step is potentially optional.

Index PCR Reaction:

After addition of the first and second adapters, samples are indexed for sequencing using the following conditions.

TABLE 36
Reaction Mixture for Indexing PCR Reaction

| | Per Sample | Initial | Final |
|---|---|---|---|
| Reagent | (μL) | concentration | concentration |
| Kapa HiFi MM | 12.50 | 2x | 1x |
| Water | 0.50 | — | — |
| Sum | Add 13 μL to | | |
| | each reaction | | |
| DNA | 10.00 | — | <−add individually |
| i5 primer | 1.00 | 5 μM | <−add individually |
| i7 primer | 1.00 | 5 μM | <−add individually |

TABLE 37
Indexing PCR
Temperature and Time

| | |
|---|---|
| 98° C. for 2 min | |
| 98° C. for 25 sec | 20 cycles |
| 60° C. for 20 sec | |
| 72° C. for 20 sec | |
| 72° C. for 2 min | |
| 12° C. hold | |

Following indexing PCR, reactions are purified using SPRI magnetic beads at 1.8×, and eluted in 15 μL. Note that the number of cycles can be varied from 20, based on the starting concentration.

Samples are ready for high throughput sequencing, following quality control to determine concentration and size of DNA fragments, and purity.

### Example 5: Properties of Libraries Generated Using Conventional and Ligation Free Methods

High throughput sequencing libraries were generated from human and E. coli starting samples using the following protocols:
(1) the Phi29 Reverse ligation free protocol (Example 3),
(2) the Phi29 Forward ligation free protocol (Example 4)
(3) a modified version of the Example 3 protocol substituting Klenow for Phi29, and
(4) the NEBNext Ultra II DNA Library Prep Kit for Illumina (available at international.neb.com/products/e7645-nebnext-ultra-ii-dna-library-prep-kit-for-illumina#Product%20Information), which adds adapters via ligation.

In the protocol using Klenow (3), the following changes were made the protocol described in Example 3: the enzyme Klenow was used instead of Phi29, the buffer for the PCR reactions attaching the first and second adapters was changed to NEB Buffer 2, and the reaction conditions for these reactions were changed to 37° C. for one hour to accommodate the change in enzyme.

Libraries were evaluated for usability, mappability, complexity and the ability to detect rare molecules. Usability was calculated as the number of reads passing filters for read quality and length. Mappability was determined as the number of reads that mapped to a reference genome (human or E. coli), out of the number of usable reads. Complexity, or the rate of duplication, was the number of unique molecules per library. Detection ability was assayed as the ability to capture rare molecules, for examples, a target that was spiked at a low concentration into a sample with a high background concentration of other DNA.

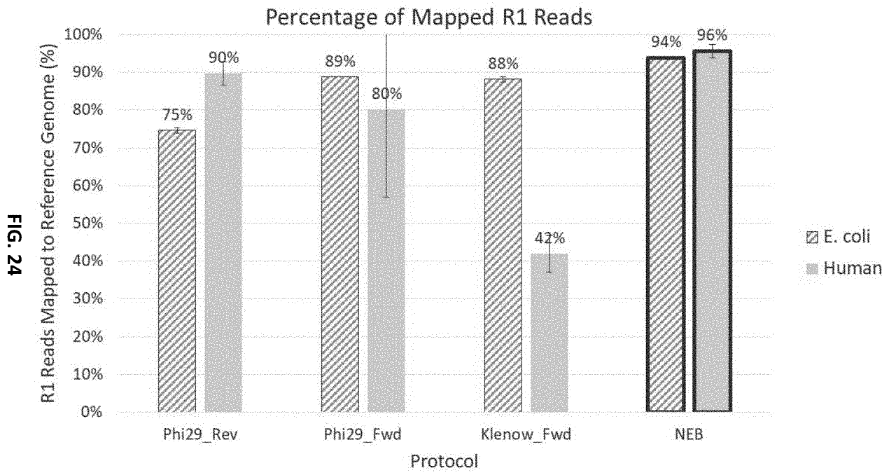

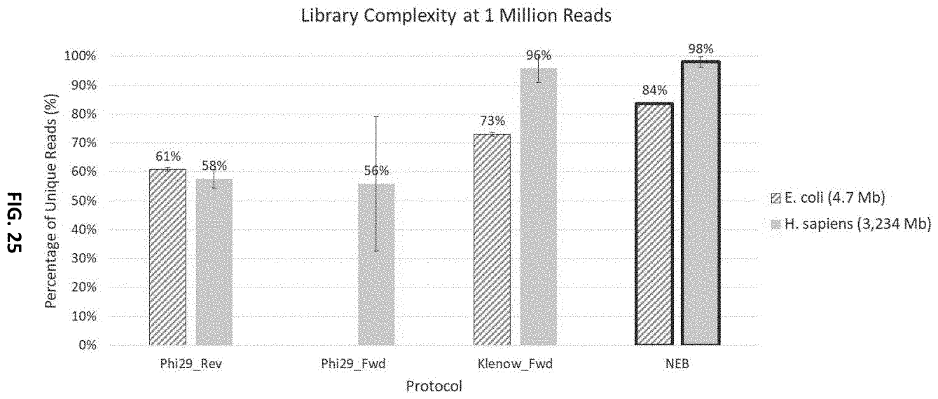

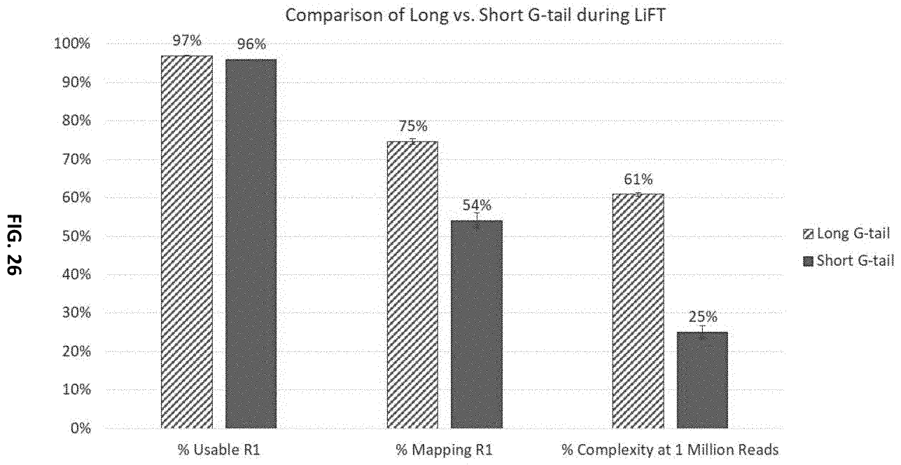

As can be seen in FIG. 23, the ligation free methods described in Examples 3 and 4 behaved comparably to the NEBNext Ultra II DNA Library Prep Kit for the percent of usable reads generated from human and E. coli starting samples. “Phi29_Rev” indicates the Phi29 Reverse protocol (as described in Example 3). “Phi29_Fwd” indicates the Phi29 Forward protocol (as described in Example 4). “Klenow_Fwd” indicates the Klenow Forward protocol (analogous to the Phi29 Forward protocol as described in Example 4, with Klenow instead of Phi29, the buffer changed to NEB Buffer 2, and the incubation temperature changed to 37° C. for 1 hour). “NEB” indicates libraries generated with the NEBNext Ultra II DNA Library Prep Kit for Illumina. Error bars indicated standard deviation based off three technical replicates, and duplicates are not removed. The results for percent mappable reads and library complexity are shown in FIG. 24 and FIG. 25, respectively.

### Example 6: Length of polyG Tail

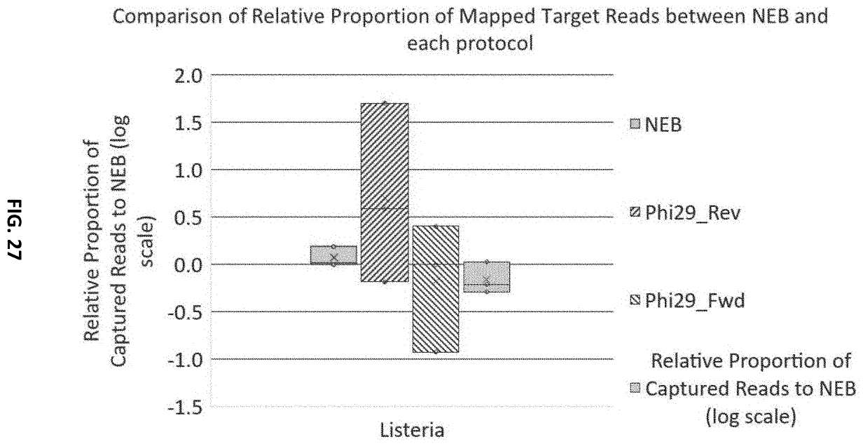

The length of the G tails was varied using the Phi29 Reverse protocol (Example 3) to generate a ligation free sequencing library from an E. coli starting sample. At the G tailing reaction step, the ratio of dGTP to 3′ ends of DNA molecules was varied from 1:1000 to generate short tails, to 1:2500 to generate long G tails. FIG. 26 shows that use of longer G-tails on nucleotide fragments during library preparation increases percent useable reads (% Useable R1), the percentage of reads that map to a reference genome (% Mapping R1) and increases library complexity (% complexity at 1 million reads). “Short G-tail” indicates libraries prepared where the ratio of 3′ ends of DNA molecules in the library to dGTPs added during the tailing reaction was 1:1000. For “Long G-tail”, this ratio was 1:2500. As can be seen in FIG. 26, longer G-tails improved the percent usable reads, the percent usable reads that mapped to a reference genome, and the complexity of the library. This is likely because the longer G-tail increases the change that the poly-C sequence on the adapter will find and hybridize with the G-tail.

### Example 7: Detection of Rare Molecules

The ability of ligation free methods of library generation to capture rare molecules in a sample was assayed. A human DNA sample was spiked with Listeria DNA at a concentration estimated to yield 1 target (Listeria) read per 833,333 human reads. This sample was used to generate high throughput sequencing libraries using the Phi29 Reverse Protocol (Example 3), the Phi29 Forward Protocol (Example 4), and the NEBNext Ultra II DNA Library Prep Kit. FIG. 27 shows the Relative Proportion of Mapped Target Reads between the NEBNext Ultra II DNA Library Prep Kit and the protocols described in Examples 3 and 4.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of preparing a library of nucleic acids, comprising:
a. providing a sample of nucleic acids comprising at least one sequence of interest;
b. contacting the sample of nucleic acids with a plurality of first polymerase chain reaction (PCR) primers, and a first polymerase under conditions that allow PCR to occur, thereby generating a plurality of first single-sided PCR products;
c. contacting the plurality of first single-sided PCR products with a terminal transferase and dNTPs under conditions sufficient to transfer dNTPs to the 3′ ends of the plurality of first single-sided PCR products, thereby generating a plurality of PCR products comprising 3′ tails; and
d. contacting the plurality of PCR products comprising 3′ tails with a plurality of second PCR primers, and the first polymerase under conditions that allow PCR to occur;
thereby generating a library of nucleic acids with adapters at the 5′ and 3′ ends.

2. The method of claim 1, comprising:
e. contacting the library of nucleic acids from (d) with a plurality of first indexing primers, a plurality of second indexing primers and a second polymerase under conditions that allow PCR to occur.

3. The method of claim 1 or 2, wherein the plurality of first PCR primers comprise (i) a sequence complementary to a sequence adjacent to or overlapping the at least one sequence of interest, and (ii) a first adapter sequence.

4. The method of claim 3, wherein the first adapter sequence is 5′ of the sequence complementary to the sequence adjacent to or overlapping the at least one sequence of interest.

5. The method of any one of claims 1-4, wherein the 3′ tail is a polyA tail, a polyG tail, a polyC tail or a polyT tail.

6. The method of any one of claims 3-5, wherein the sequence complementary to a sequence adjacent to or overlapping the at least one sequence of interest is a random sequence.

7. The method of claim 6, wherein the random sequence is a random 9mer.

8. The method any one of claims 1-7, wherein the plurality of first PCR primers comprise at least one basepair comprising a phosphorothioate linkage.

9. The method of claim 8, herein the plurality of first PCR primers comprise two 3′ and two 5′ basepairs comprising phosphorothioate linkages.

10. The method of any one of claims 1-9, wherein steps (b) and (d) comprise isothermal amplification reactions.

11. The method of claim 10, wherein the first polymerase is Phi29, Klenow exo- or Bsu DNA Polymerase, Large Fragment.

12. The method of any one of claims 1-11, wherein the plurality of second PCR primers comprise (i) a sequence complementary to the 3′ tails from step (c), and (ii) a second adapter sequence.

13. The method of claim 5, wherein the second adapter sequence is 5′ of the sequence complementary to the 3′ tail.

14. The method of claim 12 or 13, wherein the 3′ tails comprise polyG tails, and wherein the sequence complementary to the 3′ tail comprises polyC.

15. The method any one of claims 1-14, wherein the plurality of second PCR primers comprise at least one basepair comprising a phosphorothioate linkage.

16. The method of claim 15, herein the plurality of second PCR primers comprise two 3′ and two 5′ basepairs comprising phosphorothioate linkages.

17. The method of any one of claims 1-16, wherein step (b) and/or (d) is carried out in an emulsion.

18. The method of any one of claims 2-17, wherein first indexing primers comprise a sequence complementary to the first adapter and a first unique molecular identifier sequence (UMI).

19. The method of any one of claims 2-18, wherein the second indexing primers comprise a sequence complementary to the second adapter and a second UMI sequence.

20. The method of any one of claims 1-19, comprising contacting the sample of nucleic acids with a first enzyme prior to step (b) under conditions that allow for blunting of overhangs in the sample of nucleic acids, thereby generating a blunt-ended sample of nucleic acids.

21. The method of claim 20, wherein the first enzyme comprises T4 polymerase, Klenow fragment, or Mung Bean Nuclease.

22. The method of claim 21, comprising purifying the blunt-ended sample of nucleic acids.

23. The method of claim 22, wherein the purifying comprises removing unincorporated dNTPs.

24. The method of claim 23, wherein removing unincorporated dNTPs comprises treating with recombinant shrimp alkaline phosphatase (rSAP), purification using a column or bead-based purification.

25. The method of any one of claims 20-24, comprising contacting the blunt-ended sample of nucleic acids with a second enzyme under conditions that allow for the addition of dideoxynucleotides (ddNTPs) to the to the 3′ ends of the blunt ended nucleic acids in the sample, and wherein contacting the blunt-ended sample of nucleic acids with the second enzyme occurs prior to step (b).

26. The method of claim 25, wherein the second enzyme has 3′ to 5 exonuclease activity and polymerase activity but does not have 5′ to 3′ exonuclease activity.

27. The method of claim 26, wherein the second enzyme comprises a Klenow fragment.

28. The method of claim 27, comprising purifying the blunt-ended sample of nucleic acids after contacting the blunt-ended sample of nucleic acids with the second enzyme.

29. The method of any one of claims 1-28, comprising purifying the plurality of first single-sided PCR products following step (b).

30. The method of any one of claims 1-29, comprising purifying the plurality of first single-sided PCR products following step (b) and prior to step (c).

31. The method of any one of claims 1-30, comprising purifying the plurality of PCR products comprising 3′ tails after step (c) and prior to step (d).

32. The method of any one of claims 1-31, comprising purifying the plurality of PCR products from (d).

33. The method of any one of claims 32-32, wherein the purifying comprises removing unincorporated dNTPs or ddNTPs.

34. The method of any one of claims 32-33, wherein removing unincorporated ddNTPs comprises treating with recombinant shrimp alkaline phosphatase (rSAP), purification using a column, or bead-based purification.

35. The method of claim 34, wherein the bead-based purification comprises SPRI magnetic beads.

36. The method of any one of claims 1-35, wherein the nucleic acids comprise ribonucleic acids (RNAs), deoxyribonucleic acids (DNAs), or a combination thereof.

37. The methods of any one of claims 18-35, wherein the first unique molecular identifier sequence (UMI) comprises 2, 3, 4, 5, 6, 7, 8, 9, 10, 11 or 12 nucleotides.

38. The method of claim 37, wherein the first UMI is a random sequence.

39. The method of any one of claims 1-38, wherein the first adapter comprises a sequence of a first sequencing adapter.

40. The method of claim 39, wherein the first adapter comprises a sequence of ACACTCTTTCCCTACACGACGCTCTTCCGATCT (SEQ ID NO:4).

41. The method of any one of claims 19-040, wherein the second UMI comprises 2, 3, 4, 5, 6, 7, 8, 9, 10, 11 or 12 nucleotides.

42. The method of claim 41, wherein the second UMI is a random sequence.

43. The method of any one of claims 1-42, wherein the second adapter comprises a sequence of a second sequencing adapter.

44. The method of claim 43, wherein the second adapter comprises a sequence of GACTGGAGTTCAGACGTGTGCTCTTCCGATCT (SEQ ID NO:3).

45. The method of any one of claims 1-484 wherein the sequence adjacent to or overlapping the sequence of interest is within 1-500, 1-300, 1-200, 1-100, 1-75, 1-50 or 1-25 nucleotides of the sequence of interest.

46. The method of any one of claims 1-45, wherein the sequence adjacent to or overlapping the sequence of interest is within 1-25 nucleotides of the sequence of interest.

47. The method of any one of claims 1-56, wherein the sequence of interest comprises a single nucleotide polymorphism (SNP), a miniSTR (mini short tandem repeat), a mitochondrial marker, a Y chromosome marker, a taxonomic marker, or a disease trait marker.

48. The method of claim 47, wherein the disease trait marker comprises a marker for pathogenicity, virulence, resistance or strain identification.

49. The method of any one of claims 1-48, wherein the sample is degraded.

50. The method of any one of claims 1-49, wherein the sample is a forensics sample.

51. The method of any one of claims 1-50, comprising sequencing the library of nucleic acids.

52. The methods of any one of claims 1-51, wherein the at least one sequence of interest comprises at least 1, 2, 3, 4, 5, 6, 7, 8, 9, 10, 15, 20, 25, 30, 40, 50, 60, 70, 80, 90, 100, 500, 1000, 10,000, 50,000, 100,000 or 200,000 unique sequences of interest.

53. The method of any one of claims 1-52, comprising sequencing the library of nucleic acids.

54. The method of claim 53, wherein the sequencing is high-throughput sequencing.

55. A method of preparing a library of nucleic acids, comprising:
a. providing a sample of nucleic acids comprising at least one sequence of interest;
b. contacting the sample of nucleic acids with a terminal transferase and dNTPs under conditions sufficient to transfer dNTPs to the 3′ ends of the nucleic acids, thereby generating a sample of nucleic acids comprising 3′ tails;
c. contacting the sample of nucleic acids comprising 3′ tails with a plurality of first polymerase chain reaction (PCR) primers, and a first polymerase under conditions that allow PCR to occur, thereby generating a plurality of first single-sided PCR products; and
d. contacting the plurality of first single-sided PCR products with a plurality of second PCR primers, and the first polymerase under conditions that allow PCR to occur;
thereby generating a library of nucleic acids with adapters at the 5′ and 3′ ends.

56. The method of claim 55, comprising:
e. contacting the library of nucleic acids from (d) with a plurality of first indexing primers, a plurality of second indexing primers and a second polymerase under conditions that allow PCR to occur.

57. The method of claim 55 or 56, wherein the 3′ tail is a polyA tail, a polyG tail, a polyC tail or a polyT tail.

58. The method of any one of claims 55-57, wherein the plurality of first PCR primers comprise (i) a sequence complementary to the 3′ tails from step (c), and (ii) a first adapter sequence.

59. The method of claim 58, wherein the first adapter sequence is 5′ of the sequence complementary to the 3′ tail.

60. The method of any one of claims 55-59, wherein the 3′ tails comprise polyG tails, and wherein the sequence complementary to the 3′ tail comprises polyC.

61. The method any one of claims 55-60, wherein the plurality of first PCR primers comprise at least one basepair comprising a phosphorothioate linkage.

62. The method of claim 65, herein the plurality of first PCR primers comprise two 3′ and two 5′ basepairs comprising phosphorothioate linkages.

63. The method of any one of claims 55-62, wherein the plurality of second PCR primers comprise (i) a sequence complementary to a sequence adjacent to or overlapping the at least one sequence of interest, and (ii) a second adapter sequence.

64. The method of claim 63, wherein the second adapter sequence is 5′ of the sequence complementary to the sequence adjacent to or overlapping the at least one sequence of interest.

65. The method of claim 63 or 64, wherein the sequence complementary to a sequence adjacent to or overlapping the at least one sequence of interest is a random sequence.

66. The method of claim 65, wherein the random sequence is a random 9mer.

67. The method any one of claims 55-56, wherein the plurality of second PCR primers comprise at least one basepair comprising a phosphorothioate linkage.

68. The method of claim 67, wherein the plurality of second PCR primers comprise two 3′ and two 5′ basepairs comprising phosphorothioate linkages.

69. The method of any one of claims 55-68, wherein steps (c) and (d) comprise isothermal amplification reactions.

70. The method of claim 69, wherein the first polymerase is Phi29, Klenow exo- or Bsu DNA Polymerase, Large Fragment.

71. The method of any one of claims 55-70, comprising purifying the sample of nucleic acids comprising 3′ tails produced at step (b).

72. The method of any one of claims 55-71, comprising purifying the plurality of first single-sided PCR products produced at step (c).

73. The method of any one of claims 55-72, comprising purifying the library of nucleic acids with adapters at the 5′ and 3′ ends produced at step (d).

74. The method of any one of claims 73-73, wherein purifying the plurality of first single-sided PCR products comprises treating with recombinant shrimp alkaline phosphatase (rSAP), purification using a column or bead-based purification.

75. The method of claim 74, wherein the beads are SPRI magnetic beads.

76. The method of any one of claims 55-75, wherein step (b) is carried out in an emulsion.

77. The method of any one of claims 55-76, wherein step (c) is carried out in an emulsion.

78. The method of any one of claims 56-77, wherein first indexing primers comprise a sequence complementary to the first adapter and a first unique molecular identifier sequence (UMI).

79. The method of any one of claims 56-78, wherein the second indexing primers comprise a sequence complementary to the second adapter and a second UMI sequence.

80. The method of any one of claims 56-79, wherein the nucleic acids comprise ribonucleic acids (RNAs), deoxyribonucleic acids (DNAs), or a combination thereof.

81. The methods of any one of claims 78-80, wherein the first unique molecular identifier sequence (UMI) comprises 2, 3, 4, 5, 6, 7, 8, 9, 10, 11 or 12 nucleotides.

82. The method of claim 81, wherein the first UMI is a random sequence.

83. The method of any one of claims 55-82, wherein the first adapter comprises a sequence of a first sequencing adapter.

84. The method of any one of claims 79-83, wherein the second UMI comprises 2, 3, 4, 5, 6, 7, 8, 9, 10, 11 or 12 nucleotides.

85. The method of claim 84, wherein the second UMI is a random sequence.

86. The method of any one of claims 55-85, wherein the second adapter comprises a sequence of a second sequencing adapter.

87. The method of any one of claims 55-86, wherein the sequence adjacent to or overlapping the sequence of interest is within 1-500, 1-300, 1-200, 1-100, 1-75, 1-50 or 1-25 nucleotides of the sequence of interest.

88. The method of any one of claims 55-87, wherein the sequence adjacent to or overlapping the sequence of interest is within 1-25 nucleotides of the sequence of interest.

89. The method of any one of claims 55-88, wherein the sequence of interest comprises a single nucleotide polymorphism (SNP), a miniSTR (mini short tandem repeat), a mitochondrial marker, a Y chromosome marker, a taxonomic marker, or a disease trait marker.

90. The method of claim 89, wherein the disease trait marker comprises a marker for pathogenicity, virulence, resistance or strain identification.

91. The method of any one of claims 55-90, wherein the sample is degraded.

92. The method of any one of claims 55-91, wherein the sample is a forensics sample.

93. The method of any one of claims 55-92, comprising sequencing the library of nucleic acids.

94. The methods of any one of claims 55-93, wherein the at least one sequence of interest comprises at least 1, 2, 3, 4, 5, 6, 7, 8, 9, 10, 15, 20, 25, 30, 40, 50, 60, 70, 80, 90, 100, 500, 1000, 10,000, 50,000, 100,000 or 200,000 unique sequences of interest.

95. The method of any one of claims 55-94, comprising sequencing the library of nucleic acids.

96. The method of claim 95, wherein the sequencing is high-throughput sequencing.

97. The methods of any one of claims 1-96, comprising:
a. providing a plurality of guide nucleic acid (gNA)-CRISPR/Cas system protein complexes, wherein the gNAs are configured to hybridize to at least one sequence targeted for depletion;
b. mixing the library of nucleic acids with the plurality of gNA-CRISPR/Cas system protein complexes,
wherein at least a portion of the gNA-CRISPR/Cas system protein complexes hybridize to at least one sequence targeted for depletion; and
c. incubating the mixture to cleave the at least one sequence targeted for depletion.

98. The method of claim 97, comprising PCR amplifying the library of nucleic acids following step (c).

99. The method of claim 97 or 98, wherein the CRISPR/Cas system protein comprises Cpf1, Cas9, Cas3, Cas8a-c, Cas10, CasX, CasY, Cas13, Cas14, Cse1, Csy1, Csn2, Cas4, Csm2, Cm5 or a combination thereof.

100. The method of any one of claims 97-99, wherein the CRISPR/Cas system protein comprises Cas9, Cpf1 or a combination thereof.

101. The method of any one of claims 97-99, wherein CRISPR/Cas system protein is a Cas9 or Cpf1 nickase.

102. The method of any one of claims 97-102, wherein CRISPR/Cas system protein is thermostable.

103. The method of any one of claims 97-102, wherein the gNAs are deoxyribonucleic acid (gDNAs) or ribonucleic acids (gRNAs).

104. The method of any one of claims 97-103, wherein the plurality of gNAs comprise at least 2, 10, 10², 10³, 10⁴, 10⁵ or 10⁶ unique gNAs.

105. The method of any one of claims 97-104, comprising sequencing the library of nucleic acids.

106. The method of claim 105, wherein the sequencing is high-throughput sequencing.

107. The method of any one of claims 1-106, wherein the sample comprises nucleic acids of interest and nucleic acids targeted for depletion, and at least a subset of the nucleic acids targeted for depletion comprise a plurality of recognition sites for a modification-sensitive restriction enzyme.

108. The method of claim 107, comprising terminally dephosphorylating a plurality of the nucleic acids in the sample.

109. The method of claim 107 or 108, comprising contacting the sample with the modification-sensitive restriction enzyme under conditions that allow for the cleavage of the modification-sensitive restriction sites in the nucleic acids in the sample, thereby generating nucleic acids with exposed terminal phosphates; and contacting the sample with an exonuclease under conditions that allow for the successive removal of nucleotides from a phosphorylated end of a nucleic acid; thereby generating a sample enriched for nucleic acids of interest.

110. The method of claim 109, comprising contacting the sample of nucleic acids with a modification-sensitive restriction enzyme under conditions that allow for the cleavage of the modification-sensitive restriction sites in the nucleic acids in the sample; thereby generating a sample enriched for nucleic acids of interest that have adapters on their 5′ and 3′ ends.

111. A method of preparing a library of nucleic acids, comprising
a. providing a sample of nucleic acids comprising at least one sequence of interest;
b. contacting the sample of nucleic acids with a terminal transferase and NTPs under conditions sufficient to transfer NTPs to the 3′ end of the nucleic acids thereby generating a plurality of nucleic acids comprising 3′ tails;
c. contacting the plurality of nucleic acids comprising 3′ tails with a plurality of first adapters and a reverse transcriptase under conditions sufficient for first strand complementary DNA (cDNA) synthesis to occur, thereby generating a plurality of cDNAs,
wherein the plurality of cDNAs comprise 3′ polyC sequences; and
d. contacting the plurality of cDNAs with a second adapter under conditions sufficient to allow generation of double stranded DNA from the plurality of cDNAs to generate a plurality of double stranded DNAs,
thereby preparing a library of nucleic acids with adapters at the 5′ and 3′ ends.

112. The method of claim 111, wherein the plurality of first adapters comprise a sequence complementary to the 3′ tails and a first UMI sequence.

113. The method of claim 111 or 112, wherein the plurality of second adapters comprise a second UMI and a polyG sequence.

114. The method of any one of claims 111-113, wherein the nucleic acids comprise ribonucleic acids (RNAs).

115. The method of any one of claims 111-114, wherein the reverse transcriptase comprises Moloney Murine Leukemia Virus (MMLV) reverse transcriptase.

116. The method of claim 111, wherein step (d) comprises adding a polymerase.

117. The method of claim 116, wherein step (d) comprises PCR amplification of the plurality of double stranded DNAs.

118. The methods of any one of claims 112-117, wherein the first unique molecular identifier sequence (UMI) comprises 2, 3, 4, 5, 6, 7, 8, 9, 10, 11 or 12 nucleotides.

119. The method of claim 1117, wherein the first UMI is a random sequence.

120. The method of any one of claims 111-109, wherein the first adapter comprises a sequence of a first sequencing adapter.

121. The method of any one of claims 112-120, wherein the second UMI comprises 2, 3, 4, 5, 6, 7, 8, 9, 10, 11 or 12 nucleotides.

122. The method of claim 121, wherein the second UMI is a random sequence.

123. The method of any one of claims 111-122, wherein the second adapter comprises a sequence of a second sequencing adapter.

124. The method of any one of claims 111-123, wherein the sequence of interest comprises a single nucleotide polymorphism (SNP), a miniSTR (mini short tandem repeat), a mitochondrial marker, a Y chromosome marker, or a disease trait marker.

125. The method of claim 124, wherein the disease trait marker comprises a marker for pathogenicity, virulence, resistance or strain identification.

126. The method of any one of claims 111-125, wherein the sample is degraded.

127. The method of any one of claims 111-126, wherein the sample is a forensics sample.

128. The method of any one of claims 111-127, wherein the at least one sequence of interest comprises at least 1, 2, 3, 4, 5, 6, 7, 8, 9, 10, 15, 20, 25, 30, 40, 50, 60, 70, 80, 90, 100, 500, 1000, 10,000, 50,000, 100,000 or 200,000 unique sequences of interest.

129. The method of any one of claims 111-128, wherein the sample of nucleic acids comprises ribonucleic acids (RNAs).

130. The method of claims 111-129, comprising sequencing the library of nucleic acids.

131. The method of claim 130, wherein the sequencing comprises high-throughput sequencing.

132. The methods of any one of claims 111-128, comprising:
a. providing a plurality of guide nucleic acid (gNA)-CRISPR/Cas system protein complexes, wherein the gNAs are configured to hybridize to at least one sequence targeted for depletion;
b. mixing the library of nucleic acids with the plurality of gNA-CRISPR/Cas system protein complexes,
wherein at least a portion of the gNA-CRISPR/Cas system protein complexes hybridize to the at least one sequence targeted for depletion; and
c. incubating the mixture to cleave the at least one sequence targeted for depletion.

133. The method of claim 132, comprising PCR amplifying the library of nucleic acids following step (c).

134. The method of claim 132 or 133, wherein the CRISPR/Cas system protein comprises Cpf1, Cas9, Cas3, Cas8a-c, Cas10, CasX, CasY, Cas13, Cas14, Cse1, Csy1, Csn2, Cas4, Csm2, Cm5 or a combination thereof.

135. The method of any one of claims 132-134, wherein the CRISPR/Cas system protein comprises Cas9, Cpf1 or a combination thereof.

136. The method of any one of claims 132-135, wherein CRISPR/Cas system protein is a Cas9 or Cpf1 nickase.

137. The method of any one of claims 132-136, wherein CRISPR/Cas system protein is thermostable.

138. The method of any one of claims 132-137, wherein the gNAs are deoxyribonucleic acids (gDNAs) or ribonucleic acids (gRNAs).

139. The method of any one of claims 132-138, wherein the plurality of gNAs comprise at least 2, 10, 10², 10³, 10⁴, 10⁵ or 10⁶ unique gNAs.

140. The method of any one of claims 132-139, comprising sequencing the library of nucleic acids.

141. The method of claim 140, wherein the sequencing is high throughput sequencing.


SEQUENCE LISTING (27 sequences)

SEQ ID NO: 1 - DNA, 41 bp, Artificial Sequence
  PCR primer
  misc_feature (33)..(41): n is a, c, g, or t
acactctttccctacacgacgctcttccgatcnnnnnnnnn

SEQ ID NO: 2 - DNA, 37 bp, Artificial Sequence
  PCR primer
gactggagttcagacgtgtgctcttccgatccccccc

SEQ ID NO: 3 - DNA, 32 bp, Artificial Sequence
  adapter sequence
gactggagttcagacgtgtgctcttccgatct

SEQ ID NO: 4 - DNA, 33 bp, Artificial Sequence
  adapter sequence
acactctttccctacacgacgctcttccgatct

SEQ ID NO: 5 - DNA, 41 bp, Artificial Sequence; identical to SEQ ID NO: 1
  PCR primer
  misc_feature (33)..(41): n is a, c, g, or t

SEQ ID NO: 6 - DNA, 22 bp, Artificial Sequence
  adapter sequence
gttttagagctatgctgttttg

SEQ ID NO: 7 - DNA, 86 bp, Artificial Sequence
  encodes tracrRNA
ggaaccattcaaaacagcatagcaagttaaaataaggctagtccgttatcaacttgaaaaagtggcaccgagtcggtgcttttttt

SEQ ID NO: 8 - DNA, 83 bp, Artificial Sequence
  gNA sequence on one strand
gttttagagctagaaatagcaagttaaaataaggctagtccgttatcaacttgaaaaagtggcaccgagtcggtgcttttttt

SEQ ID NO: 9 - DNA, 83 bp, Artificial Sequence
  reverse complement of gNA strand sequence
aaaaaaagcaccgactcggtgccactttttcaagttgataacggactagccttattttaacttgctatttctagctctaaaac

SEQ ID NO: 10 - DNA, 83 bp, Artificial Sequence; identical to SEQ ID NO: 9
  gNA sequence

SEQ ID NO: 11 - RNA, 83 nt, Artificial Sequence
  gNA stem-loop sequence
guuuuagagcuagaaauagcaaguuaaaauaaggcuaguccguuaucaacuugaaaaaguggcaccgagucggugcuuuuuuu

SEQ ID NO: 12 - DNA, 94 bp, Artificial Sequence
  gNA stem-loop
gttttagagctatgctggaaacagcatagcaagttaaaataaggctagtccgttatcaacttgaaaaagtggcaccgagtcggtgctttttttc

SEQ ID NO: 13 - DNA, 94 bp, Artificial Sequence
  gNA stem-loop reverse complement
gaaaaaaagcaccgactcggtgccactttttcaagttgataacggactagccttattttaacttgctatgctgtttccagcatagctctaaaac

SEQ ID NO: 14 - DNA, 94 bp, Artificial Sequence; identical to SEQ ID NO: 13
  gNA stem-loop

SEQ ID NO: 15 - RNA, 94 nt, Artificial Sequence
  gNA stem-loop
guuuuagagcuaugcuggaaacagcauagcaaguuaaaauaaggcuaguccguuaucaacuugaaaaaguggcaccgagucggugcuuuuuuuc

SEQ ID NO: 16 - RNA, 19 nt, Artificial Sequence
  gNA sequence
aauuucuacuguuguagau

SEQ ID NO: 17 - DNA, 19 bp, Artificial Sequence
  gNA
aatttctactgttgtagat

SEQ ID NO: 18 - RNA, 83 nt, Artificial Sequence; identical to SEQ ID NO: 11
  gRNA

SEQ ID NO: 19 - RNA, 94 nt, Artificial Sequence; identical to SEQ ID NO: 15
  gRNA

SEQ ID NO: 20 - RNA, 22 nt, Artificial Sequence
  tracrRNA
guuuuagagcuaugcuguuuug

SEQ ID NO: 21 - RNA, 86 nt, Artificial Sequence
  tracrRNA
ggaaccauucaaaacagcauagcaaguuaaaauaaggcuaguccguuaucaacuugaaaaaguggcaccgagucggugcuuuuuuu

SEQ ID NO: 22 - RNA, 19 nt, Artificial Sequence
  gRNA stem-loop
aauuucuacuguuguagau

SEQ ID NO: 23 - RNA, 19 nt, Artificial Sequence
  Cpf1 gRNA stem-loop
aauuucuacuguuguagau

SEQ ID NO: 24 - DNA, 37 bp, Artificial Sequence; identical to SEQ ID NO: 2
  adapter sequence (Ad2)

SEQ ID NO: 25 - DNA, 37 bp, Artificial Sequence; identical to SEQ ID NO: 2
  Ad2 sequence with phosphorothioates

SEQ ID NO: 26 - DNA, 41 bp, Artificial Sequence; identical to SEQ ID NO: 1
  adapter sequence (Ad1)
  misc_feature (33)..(41): n is a, c, g, or t

SEQ ID NO: 27 - DNA, 41 bp, Artificial Sequence; identical to SEQ ID NO: 1
  Ad1 sequence with phosphorothioates
  misc_feature (33)..(41): n is a, c, g, or t